(19) United States
(12) Patent Application Publication
(10) Pub. No.: US 2025/0215499 A1
(43) Pub. Date: Jul. 3, 2025

(54) TUMOR SUPPRESSOR SP0495

(71) Applicant: The Chinese University of Hong Kong, Shatin (CN)

(72) Inventors: Qian TAO, Shatin (CN); Lili LI, Shatin (CN); Xingsheng SHU, Shatin (CN)

(21) Appl. No.: 18/403,634

(22) Filed: Jan. 3, 2024

(51) Int. Cl. C12Q 1/6886; A61K 38/17; A61P 35/00; G01N 33/574

(52) U.S. Cl. CPC C12Q 1/6886; A61K 38/17; A61P 35/00; G01N 33/574; C12Q 2600/118; C12Q 2600/154; C12Q 2600/158

(57) ABSTRACT

The present invention relates to the tumor suppressor SP0495, the expression of which is suppressed in cancers due to increased methylation in the genomic sequence of the promoter region controlling its expression, and therefore provides methods for diagnosing cancer, determining cancer risk, and prognosing cancer mortality in a subject by assessing the level of SP0495 expression in the affected cells or tissues. A kit and device useful for such methods are also provided. In addition, the present invention provides a method for treating cancer by increasing SP0495 expression or activity.

### BACKGROUND OF THE INVENTION

Cancer-related causes are among the top reasons of death in developed countries. In the US alone, the number of new cases of cancer of any site averages about 450 per 100,000 men and women per year, and the number of deaths averages about 170 per 100,000 men and women per year. Cancer is a disease with a high mortality rate: while about 1,700,000 newly diagnosed cancer cases are expected each year, over 600,000 deaths annually are attributable to various types of cancer. In 2020, for example, over 1.8 million new cancer cases were diagnosed and over 600,000 cancer deaths were recorded. Based on data from recent years, it is estimated that over 38% of men and women will be diagnosed with cancer at some point during their lifetime.

The most common cancers in men are prostate, lung, and colorectal cancers, accounting for an estimated 43% of all new cancer diagnoses, whereas for women, the most common cancers are breast, lung, and colorectal cancers, accounting for an estimated 50% of all new cancer diagnoses. Besides the human toll in suffering and deaths, the financial toll for cancer care is enormous with an annual cost estimate in the range of $150-180 billion in the US each year. Early detection of cancers is key to successful treatment and desirable clinical outcomes, as many localized cancers can be cured with early treatment such as surgical intervention. There remains, however, a significant unmet clinical need to identify patients at an early stage, thereby allowing treatment of localized disease and hence reduced cancer mortality as well as cancer care expenditures.

Because of the prevalence of cancer and its enormous social and economical impact globally, there exists an urgent need for new and more effective, and preferably less or non-invasive, methods to diagnose, monitor, treat, and prognose cancer, including for cancer risk assessment and early detection of cancer. This invention fulfills this and other related needs.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Feb. 20, 2024 is entitled “080015-1413724-040000US_ST26.xml”, and is 72,497 bytes in size.

### BRIEF SUMMARY OF THE INVENTION

The present inventors have identified SP0495, a small protein encoded by the open reading frame 2 (ORF2) of the 1p36.3 gene KIAA0495, a gene previously thought as encoding only a long non-coding RNA (lncRNA), as a novel tumor suppressor and thus diagnostic and prognostic marker for various types of human cancer, such as colorectal cancer, gastric cancer, breast cancer, esophageal cancer, nasopharyngeal cancer, head and neck cancer, bladder cancer, cervical cancer, and lymphomas including Hodgkin lymphoma and non-Hodgkin lymphoma. More specifically, the inventors show that, compared with normal individuals, CpG islands of the promoter region genomic gene are hypermethylated in biological samples of cancer tissues taken from cancer patients, in direct contrast to non-cancerous healthy tissues where methylation of the CpGs is sparse if present at all. Such hypermethylation leads to SP0495 silencing at both mRNA and protein levels. Re-expression of SP0495 inhibits cancer cell growth and induces programmed cell death. Protein/mRNA expression level of SP0495 and promoter methylation level of KIAA0495 genomic sequence closely correlate with the survival of cancer patients and are therefore also useful as prognostic markers for cancer.

Thus, in the first aspect, the present invention provides a method for (1) assessing risk for later developing cancer in a subject who may not have exhibited any symptoms of cancer, or (2) diagnosing cancer in a patient who has manifested one or more clinical symptoms suspected of cancer. The method includes these steps: (a) measuring expression level of SP0495 in a sample taken from the subject; (b) comparing the expression level obtained in step (a) with a standard control; and (c) determining the subject, who has a reduced SP0495 expression level compared with the standard control, as having an increased risk for cancer.

In some embodiments, the sample used for practicing the method is a esophageal epithelial tissue sample. In some embodiments, the expression level of SP0495 is SP0495 protein level. In some embodiments, the expression level of SP0495 is SP0495 mRNA level. In some embodiments, step (a) comprises an immunoassay using an antibody that specifically binds the SP0495 protein; or step (a) may comprise an amplification reaction, such as a polymerase chain reaction (PCR), especially a reverse transcriptase-PCR (RT-PCR). In some embodiments, step (a) comprises a polynucleotide hybridization assay, such as a Southern Blot analysis or Northern Blot analysis, or an in situ hybridization assay. In some embodiments, when the subject is indicated as having an increased risk for cancer, the method is further includes repeating step (a) at a later time using the sample type of sample from the subject, wherein an increase in the expression level of SP0495 at the later time as compared to the amount from the original step (a) indicates a lessened risk of cancer, and a decrease indicates a heightened risk for cancer. In some cases, the cancer is colorectal cancer, gastric cancer, breast cancer, esophageal cancer, nasopharyngeal cancer, head and neck cancer, bladder cancer, cervical cancer, or lymphoma such as Hodgkin lymphoma and non-Hodgkin lymphoma. In some cases, the cancer is not lung, liver, renal, ovarian, prostate, or brain cancer. In some cases, the cancer is not ovarian cancer or prostate cancer.

In the second aspect, the present invention provides a method for assessing risk for later developing cancer in a subject who may not have exhibited any symptoms of cancer, or (2) diagnosing cancer in a patient who has manifested one or more clinical symptoms suspected of cancer. The method includes these steps: (a) treating DNA from an esophageal epithelial tissue sample taken from the subject with an agent that differentially modifies methylated and unmethylated DNA; (b) determining number of methylated CpGs in a genomic sequence, which is SEQ ID NO:3 or a fragment thereof comprising at least 10 CpGs or 15, 20, 25, 30, or more CpGs, and (c) comparing the number of methylated CpGs from step (b) with the number of methylated CpGs in the genomic sequence from a non-cancer sample and processed through steps (a) and (b); and (d) determining the subject, whose sample contains more methylated CpGs in the genomic sequence determined in step (b) compared to the number of methylated CpGs with the number of methylated CpGs in the genomic sequence from a non-cancer sample and processed through steps (1) to (3), as having an increased risk for cancer compared with a healthy subject not diagnosed with cancer or with known heightened risk for later developing cancer.

In some embodiments, the genomic sequence is SEQ ID NO:3. In embodiments, the agent that differentially modifies methylated DNA and unmethylated DNA is an enzyme that preferentially cleaves methylated DNA, an enzyme that preferentially cleaves unmethylated DNA, or a bisulfite. In some embodiments, step (b) comprises an amplification reaction, such as a PCR. In some cases, the cancer is colorectal cancer, gastric cancer, breast cancer, esophageal cancer, nasopharyngeal cancer, head and neck cancer, bladder cancer, cervical cancer, or lymphoma such as Hodgkin lymphoma and non-Hodgkin lymphoma. In some cases, the cancer is not lung, liver, renal, ovarian, prostate, or brain cancer. In some cases, the cancer is not ovarian cancer or prostate cancer.

In the third aspect, the present invention provides a method for assessing likelihood of mortality from cancer in a cancer patient. The method comprises the steps of: (a) treating DNA from a cancer tissue sample taken from a first cancer patient with an agent that differentially modifies methylated and unmethylated DNA; (b) determining number of methylated CpGs in a genomic sequence, which is SEQ ID NO:3 or a fragment thereof comprising at least 10 CpGs or 15, 20, 25, 30, or more CpGs, and (c) comparing the number of methylated CpGs from step (b) with the number of methylated CpGs in the genomic sequence from another cancer tissue sample of the same type obtained from a second patient who has been diagnosed with the same kind of cancer and processed through steps (a) and (b); and (d) determining the first patient, whose cancer tissue sample contains more methylated CpGs in the genomic sequence determined in step (b) compared to the number of methylated CpGs with the number of methylated CpGs in the genomic sequence from the same kind of cancer tissue sample obtained from the second patient suffering from the same cancer and processed through steps (1) to (3), as having an increased likelihood of mortality from the cancer compared with the second patient.

In some embodiments, the genomic sequence is SEQ ID NO:3. In some embodiments, the agent that differentially modifies methylated DNA and unmethylated DNA is an enzyme that preferentially cleaves methylated DNA, an enzyme that preferentially cleaves unmethylated DNA, or a bisulfite. In some embodiments, step (b) comprises an amplification reaction such as a PCR. In some cases, the cancer is colorectal cancer, gastric cancer, breast cancer, esophageal cancer, nasopharyngeal cancer, head and neck cancer, bladder cancer, cervical cancer, or lymphoma such as Hodgkin lymphoma and non-Hodgkin lymphoma. In some cases, the cancer is not lung, liver, renal, ovarian, prostate, or brain cancer. In some cases, the cancer is not ovarian cancer or prostate cancer.

In the fourth aspect, the present invention provides a kit for detecting cancer in a subject. The kit includes (1) a standard control that provides an average amount of SP0495 protein or SP0495 mRNA; and (2) an agent that specifically and quantitatively identifies SP0495 protein or SP0495 mRNA. In some embodiments, the agent in (2) is an antibody that specifically binds the SP0495 protein. In some embodiments, the agent in (2) is a polynucleotide probe that hybridizes with the SP0495 mRNA. In some embodiments, the agent comprises a detectable moiety. In some embodiments, the kit may further include two oligonucleotide primers for specifically amplifying at least a segment of SEQ ID NO:3, which contains at least 10, possibly 15, 20, 25, 30, or more CpGs, or its complement in an amplification reaction (e.g., a PCR). Optionally, the kit in some cases may further include an instruction manual to provide instructions for the users.

In the fifth aspect, the present invention provides a method for inhibiting growth of an cancer cell, comprising contacting or introducing into the cancer cell with an effective amount of a polypeptide comprising the amino acid sequence set forth in SEQ ID NO:1 or a nucleic acid comprising a polynucleotide sequence encoding SEQ ID NO:1. In some embodiments, the nucleic acid is an expression cassette comprising a promoter (e.g., a promoter directing protein expression in a specific cell or tissue type) operably linked to the polynucleotide sequence encoding SEQ ID NO:1. In some embodiments, the nucleic acid comprises the polynucleotide sequence set forth in SEQ ID NO:2. In some embodiments, the method is practiced to inhibit the growth of cancer cells within a patient's body, when the patient may or may not have exhibited clinical symptoms of cancer. In some cases, the cancer is colorectal cancer, gastric cancer, breast cancer, esophageal cancer, nasopharyngeal cancer, head and neck cancer, bladder cancer, cervical cancer, or lymphoma such as Hodgkin lymphoma and non-Hodgkin lymphoma. In some cases, the cancer is not lung, liver, renal, ovarian, prostate, or brain cancer. In some cases, the cancer is not ovarian cancer or prostate cancer.

### BRIEF DESCRIPTION OF THE DRAWINGS

FIG. 1A-FIG. 1G. Epigenomic identification of KIAA0495 as a novel 1p36.3 TSG candidate. (FIG. 1A) CpG methylome analysis by MeDIP-chip detected signal enrichment in the KIAA0495 promoter CGI in carcinoma cell lines of colorectal (CRC), gastric (GsCa), head and neck (HNC), breast (BrCa) and immortalized normal mammary cell line HMEpC. Positive signal peaks (red) and the exon 1 of KIAA0495 are marked. (FIG. 1B) Schematic diagram of KIAA0495 transcript variants. Blank boxes represent exons gapped with lines (introns), blue boxes represent the predicted largest ORF2 of KIAA0495. Positions of RT-PCR primers (F/R) are denoted by arrows. (FIG. 1C) Broad expression of KIAA0495 in human normal adult tissues and fetal tissues by semi-quantitative RT-PCR, with GAPDH as a control. Sk. muscle, skeletal muscle. (FIG. 1D) Average R values of all CpG sites located at the KIAA0495 promoter were plotted for CRC, ESCA, and BRCA primary tumors and adjacent normal samples from the TCGA database. Statistical analysis is performed by one-way ANOVA. (FIG. 1E) KIAA0495 expression levels in different tumor types from TCGA database, as determined by TIMER2.0 (*, p<0.05; ***, p<0.001). (FIG. 1F) Analyses of TCGA datasets reveal an inverse correlation between KIAA0495 mRNA expression and its promoter methylation in CRC, ESCA, and BRCA. Each green circle represents a single clinical sample. Pearson correlation coefficient analysis is used. (FIG. 1G) Kaplan-Meier curve analyses show the overall survival of patients with colorectal, esophageal, lung and breast cancers in GSE and TCGA datasets by log-rank test.

FIG. 2A-FIG. 2H. KIAA0495 encodes a small protein SP0495. (FIG. 2A) Predicted ORFs within the transcript 1 of KIAA0495 (NR_033711.1). The largest ORF—ORF2 with protein-coding potential is marked with a green arrow. As shown in the bottom panel, there is a stop codon (TGA) located right upstream of the start codon (ATG) of ORF2. (FIG. 2B) Genomic location of the predicted common ORF2 at the four KIAA0495 transcripts. Boxes represent exons gapped with lines (introns), and filled green boxes represent ORF2. RNA-seq (green) and Ribo-seq (red) coverage plots at the KIAA0495 locus from aggregated human data (GWIPS-viz) are shown. Locations of elongating ribosomes (red) generated are consistent with the coding regions of KIAA0495-ORF2. The isolated peaks of ribosome profiling density represent ribosome stalling sites. (FIG. 2C) Diagram of the IRES fusion constructs of KIAA0495-ORF2. TGA-KIAA0495-ORF2 represents a construct with an additional 5′-UTR fragment added to the upstream of KIAA0495-ORF2 (upper panel). In vitro protein translation of KIAA0495-ORF2 yields a small protein SP0495 at the predicted size (˜21 kDa). Asterisks indicate bands of the SP0495 protein at the correct size (lower panel). (FIG. 2D) Western blot detects ectopic expression of KIAA0495-ORF2/SP0495 protein in HCT116 and KYSE150 cells. α-Tubulin was used as a loading control. Representative data are shown. (FIG. 2E) Endogenous SP0495 protein is detected in multiple human normal tissues by Western blot using an anti-SP0495 antibody. Representative data are shown. (FIG. 2F) Immunofluorescence showing the subcellular localization of SP0495 in HONE1 (endogenous) and KYSE150 (exogenous) cells, using anti-SP0495 antibody (Ab) or anti-Flag antibody (green). Nuclei counterstained with DAPI (blue). Original magnification, 400×. Scale bar 10 uM. HONE1 photos were taken by confocal microscopy. Representative images are shown. (FIG. 2G) Co-localization of SP0495 with ER/Golgi compartments. Diagram of the SP0495 protein domain analyzed by bioinformatics is shown on the top panel. Blue box represents the location of a transmembrane & signal peptide domain (1-18-aa). KYSE150 cells with SP0495 expression were co-transfected with either ER- or Golgi-specific plasmid (pDsRed-ER or pEYFP-Golgi, respectively). Nuclei counterstained with DAPI (blue). Original magnification, 400×. Scale bar 10 uM. Representative images are shown. (FIG. 2H) Representative IHC images of SP0495 expression in gastric and colorectal tumors and adjacent normal tissues. SP0495 expression level is lower in gastric and colorectal tumor tissues compared to their adjacent normal tissues, with statistical significance by Student's two-tailed unpaired t-test analysis (p<0.001).

FIG. 3A-FIG. 3I. Promoter CpG methylation silences KIAA0495 in multiple tumors. (FIG. 3A) A typical CpG island (CGI) spans the promoter to exon 1 of KIAA0495. Each vertical bar represents a single CpG site. The transcription start site is indicated by a curved arrow. MSP sites and BGS region are shown. (FIG. 3B) Silencing of KIAA0495 in tumor cell lines correlated with its promoter methylation, while no methylation was detected in normal immortalized cell lines (in green underlined). CRC: colorectal cancer; HNC, head and neck cancer; ESCC: esophageal squamous cell carcinoma; Ca, carcinoma. M, methylated; U, unmethylated. (FIG. 3C) Endogenous expression of SP0495 in representative tumor cell lines by Western blot, with GAPDH as a loading control. Representative data are shown. Sample names in green underlined are normal tissues and normal immortalized cell lines. (FIG. 3D) Part of the KIAA0495 promoter CGI (−228 to +260) with 50 CpG sites was analyzed by BGS. Each row represents a single allele of the promoter analyzed, and one circle indicates one CpG site. Filled circle, methylated; Open circle, unmethylated. MSP primers (m1/m2) were indicated. DKO, DNMT1 and DNMT3B double knock-out HCT116 cell line. NPx, nasopharynx. (FIG. 3E) Pharmacological and genetic demethylation restored KIAA0495 expression in silenced cell lines of various types were examined by RT-PCR and Western blot. Representative data are shown. A+T, combined treatment using Aza and TSA. 1KO, DNMT1 knock-out, 3BKO, DNMT3B knock-out, DKO, DNMT1 and DNMT3B double knock-out. (FIG. 3F) KIAA0495 promoter is frequently methylated in primary tumor samples. Colorectal cancer (CRC) tissues (T) and paired surgical marginal non-tumor tissues (N), gastric cancer tissues, NPC and normal nasopharynx (NPx) tissues are examined by MSP. Representative data are shown. Ca, carcinoma. M, methylated; U, unmethylated. (FIG. 3G) KIAA0495 promoter methylation in representative tumor/normal pairs of CRC and ESCC tissues by BGS. Filled circle, methylated; Open circle, unmethylated. (FIG. 3H) Real-time PCR and Western blot show the downregulation of KIAA0495 in multiple primary tumors compared to matched normal tissues. Gene expression was normalized to internal control GAPDH. Data are presented as mean±SEM of three independent experiments via Student's t-test. *, p<0.05; **, p<0.01. (FIG. 3I) Association of KIAA0495 promoter methylation at specific CpG sites with patient overall survival is analyzed in rectum, esophageal, and breast metastatic tumor samples from TCGA datasets by Kaplan Meier analysis. The methylation patient groups are dichotomized by higher (β>cut-off) and lower (β<cut-off), according to a best cut-off point in MethSurv. Ca, cancer.

FIG. 4A-FIG. 4L. SP0495 is a functional tumor suppressor in carcinoma cells. Monolayer-culture (FIG. 4A) and soft-agar (FIG. 4B) colony formation assays of tumor cells. Representative data are shown with three independent experiments. (FIG. 4C) Stable expression of SP0495 in tumor cells detected by Western blot. (FIG. 4D) Quantitative analysis of colony numbers in (FIG. 4A) and (FIG. 4B), shown as ±SEM of three independent experiments using Student's t-test. *, p<0.05. (FIG. 4E) Flow cytometry analysis of apoptosis by Annexin V-FITC/PI staining of HCT116 and KYSE150 cells. Both early and late apoptotic cells (Annexin V-positive) were counted. Mean±SD of three independent experiments is shown. **, p<0.01, ***, p<0.001. (FIG. 4F) Representative results of TUNEL assay of tumor cells. Red fluorescent signals indicate TUNEL+(apoptotic) cells. Representative images are shown. Scale bar, 40 μm. (FIG. 4G) Increased cleaved PARP in SP0495-expressing HCT116 and KYSE150 cells by Western blot. (FIG. 4H) Cell cycle analysis using PI staining by flow cytometry. Representative cell cycle histograms of HCT116 and KYSE150 cells with SP0495 expression show significant increase in G1/S populations as well as significant decrease in S phase population, compared to controls. Bar diagram compares variations in cell distribution percentage in each phase of cell cycle of HCT116 and KYSE150 cells. Statistical analysis is performed by one-way ANOVA. Data represented as mean±SD of three independent experiments in triplicates. *, p<0.05; **, p<0.01. (FIG. 4I) Expression of cell cycle-related proteins in SP0495-expressing HCT116 and KYSE150 cells. Western blot analysis of phosphor-Rb/Rb, CDK4/6, phosphor-p53/p53, and p21. (FIG. 4J) Time-dependent changes in Cyclin B1 protein levels following nocodazole treatment. HCT116 and KYSE150 cells with or without SP0495 expression were treated with nocodazole (1 μM) for the length of time (hours) as indicated. Western blots were performed using indicated antibodies and GAPDH as a loading control. Summary of percentage of cells in each phase of the cell cycle with nocodazole treatment (1 μM, 24 h). Significant differences are indicated as followings: *, p<0.05; ** p<0.01 and *** p<0.001. Bars are mean±SEM from three independent experiments after Student's t-test analysis. (FIG. 4K) The activity of senescence-associated β-galactosidase in SP0495-transfected NIH3T3 cells. Representative images of NIH3T3 monolayer cultures stained for SA-β-gal activity are shown. Red arrows indicate positive staining. The percentage of SA-β-gal-positive (blue) cells is calculated (with counting >50 cells per condition) and presented as mean±SEM of three independent experiments via Student's t-test. Scale bar 10 μm. ***, p<0.001. (FIG. 4L) SP0495 suppresses colorectal tumor formation in vivo. HCT116 cells were transduced with CMV-Firefly luciferase lentivirus encoding SP0495 or empty vector, then injected subcutaneously into BALB/c nude mice. Tumor size and tumor weight were monitored. Tumor fluorescence intensity was quantified. **, p<0.01. p values determined by unbiased Student's t-test. Representative animals are shown.

FIG. 5A-FIG. 5H. SP0495 inhibits AKT/mTOR, Wnt/β-catenin and NF-kB signaling pathways. (FIG. 5A) Monolayer colony formation assay of BT549 and T47D cells treated with two different KIAA0495 siRNAs. Each bar represents mean±SEM for three independent experiments via One-Way ANOVA with post hoc analysis, with colonies (>50 cells) in empty vector-transfected cells set as 100. (FIG. 5B) Flow cytometry was performed for cell cycle analysis using PI staining. Representative cell cycle histograms of BT549 cells with SP0495 knockdown by siRNAs are shown. Bar diagram compares variations in cell distribution percentages in each phase of cell cycle of BT549 cells. Statistical analysis is performed by One-Way ANOVA with post hoc analysis. Data represented as mean±SD of three independent experiments in triplicates. (FIG. 5C) Effects of SP0495 on six key signaling pathways are assessed by luciferase reporter assays. Results are expressed as fold reduction of activity and shown as mean SEM of three independent experiments performed in triplicate analyzed by Student's t-test. *, p<0.05; **, p<0.01. (FIG. 5D) Changes of key molecules in oncogenic signaling pathways (AKT/mTOR, Wnt-β-catenin, NF-kB signaling) shown by Western blot in (D) SP0495-expression HCT116 and KYSE150 cells and (FIG. 5E) BT549 cells with KIAA0495 knockdown by siRNAs. (FIG. 5F) Real-time PCR results demonstrate that SP0495 represses multiple AKT/mTOR upstream activators (PDK1, ID1, and EEF1A2) and a panel of downstream target genes for AKT/mTOR, Wnt/β-catenin and NF-1B signaling (c-Myc, CCND1, AIP7, MITF, and TWIST1). Data are presented as mean±SEM of three independent experiments via Student's t-test. *, p<0.05; **, p<0.01. (FIG. 5G) Immunoblot analysis of c-Myc, CCND1, MMP7 expression levels, as repressed by SP0495 in HCT116 and KYSE150 cells. Representative data are shown. (FIG. 5H) Analysis of colorectal adenocarcinoma from TCGA database indicates that the mRNA expression status (Z-score) of downstream targets in AKT/mTOR, Wnt/β-catenin and NF-kB signaling pathways analyzed using cBioPortal. Right bottom: expression profiles of indicated genes in Wnt/β-catenin pathway in 32 normal colon tissues and 32 colorectal adenomas are extracted from Oncomine.

FIG. 6A-FIG. 6I. Ectopic expression of SP0495 induces autophagy in carcinoma cells. (FIG. 6A) GO enrichment analysis of differentially expressed genes identified through comparisons of SP0495-expressing tumor cells vs control cells. Q value<=0.05 is regarded as significant enrichment. (FIG. 6B) Gene set enrichment analysis (GESA) revealed significantly enriched pathways in SP0495-expressing group vs control group. NES, normalized enrichment score. (FIG. 6C) Electron microscopy images of autolysosome formation in SP0495-expressing HCT116 cells. Data are representative images of TEMs of three independent assays. Autophagosomes with double-membrane (yellow arrows) and autolysosomes with cargo in different stages of digestion (red arrows) are indicated. Scale bar, 5 μm. (FIG. 6D) Representative images detecting autophagy flux regulated by SP0495 in carcinoma cells infected with mRFP-GFP-LC3 adenovirus. Quantification of mRFP-GFP-LC3 shows that SP0495 significantly increases autophagy flux in HCT116 and KYSE150 cells. ˜50 cells are randomly selected for each statistical analysis. Data are presented as mean±SEM of three independent experiments via Student's t-test. **, p<0.01; ***, p<0.001. Scale bar, 20 μm. Original magnification, 400×. (FIG. 6E) Western blot analysis of the expression of apoptosis and autophagy proteins in HCT116 and KYSE150 cells with SP0495 expression (FIG. 6E) or empty vector (FIG. 6F) with or without BECN1 siRNA treatment. Results are representative of three independent experiments. (FIG. 6G) SP0495 facilitates cell response to autophagy by Western blot analysis of the expression of autophagy regulator proteins. HCT116 and KYSE150 cells were exposed to Torin1 (250 nM, 500 nM) for 4 hr. Results are representative of three independent experiments. (FIG. 6H) SP0495-expressing HCT116 and KYSE150 cells are treated with 10 μM BafA1 for 24 h, with LC3B and p62 levels measured by Western blot. Representative data are shown. (FIG. 6I) BafA1 suppresses colony formation in SP0495-expressing HCT116 and KYSE150 cells. Representative images are shown. Quantitative analyses of colony numbers are shown as values of mean±SEM of three independent experiments using Student's t-test, with colonies (>50 cells) of vector cells (DMSO treatment) set as 100. *, p<0.05; **, p<0.01; ***, p<0.001.

FIG. 7A-FIG. 7H. SP0495 regulates BECN1 and p62 stability and their ubiquitination in carcinoma cells. (FIG. 7A) Expression levels of BECN1 and p62 at the mRNA level in SP0495-expressing tumor cells. Bars show relative fold changes by quantitative real-time PCR (qRT-PCR) analysis. All data were normalized with GAPDH expression and given as relative to control. p values determined by Student unpaired t-test. n.s. no significant change. (FIG. 7B) Western blot examined the expression of BECN1 and p62 at the protein level in a panel of tumor cell lines with or without endogenous SP0495 expression. (FIG. 7C) Cycloheximide (CHX)-chase assay for the half-life of BECN1 and p62 in KYSE150 cells. Left panel, KYSE150 cells with SP0495 or vector expression are treated with CHX (20 g/mL) for the indicated time points, and Western blotting with indicated antibodies. Right panel, the levels of remaining BECN1 and p62 at different time points are normalized to GAPDH from three independent experiments. (FIG. 7D-G) SP0495 decreases BECN1 ubiquitination and increases the ubiquitination of p62. T-REx-293 with inducible SP0495 expression and SP0495-stable expressing KYSE150 cells were transfected with or without His-Ub plasmid and/or treatment of 10 μM MG132 for 6 h. Cell lysates were immunoprecipitated with anti-BECN1 (FIG. 7D, FIG. 7E) or anti-p62 (FIG. 7F, FIG. 7G) antibody, the precipitated materials are subjected to immunoblot for endogenous Ub (FIG. 7D, FIG. 7F) or exogenous His-Ub (FIG. 7E, FIG. 7G) to detect the ubiquitination levels of BECN1 and p62. Results are representative of three independent experiments. (FIG. 7H) BECN1 and p62 ubiquitination are performed on Ni-NTA pull-down ubiquitination analysis. HEK293 and KYSE150 cells were transfected with indicated plasmids and then treated with MG132 (10 M) for 6 h. BECN1 and p62 ubiquitination were monitored by immunoblots performed on Ni-NTA purified proteins. Results are representative of three independent experiments.

FIG. 8A-FIG. 8F. SP0495 binds to phosphoinositides with a preference for PI3P and PI(3,5)P2. FIG. 8A: Structure of the SP0495 protein predicted by Phyre2 and Swiss Model. FIG. 8B: The tertiary structure of SP0495 is predicted by Phyre2, based on the template of cllshB as a lipid-binding protein (left panel). Lipid binding domain predicted by DisoLipPred shows two lipid-protein binding motifs within SP0495 (right panel). FIG. 8C: Schematic diagram of SP0495 and its deletion mutants. SP0495-mut1 lacks the lipid-binding domain1 and SP0495-mut2 lacks the lipid-binding domain2. The in vitro expressed recombinant KIAA0495-ORF2/SP0495 protein and its two mutants are shown. FIG. 8D: Membrane and cytoplasmic fractionations of HCT116 and KYSE150 cells with or without SP0495 expression were performed. Proteins in each fraction were detected using antibodies against SP0495, INSR (membrane) and GAPDH (cytoplasm) by Western blot. FIG. 8E: Colony formation assay shows the difference in colony formation suppression abilities of SP0495 full-length and mutants. Quantitative analyses of colony numbers are shown as values of mean±SEM of three independent experiments using One-Way ANOVA with post hoc analysis. Colonies (>50 cells) in empty vector-transfected cells set as 100. FIG. 8F: Effects of SP0495 lipid binding domain deletions on cell apoptosis by flow cytometry analysis via Annexin V-FITC/PI staining. Both early and late apoptotic cells (Annexin V-positive) were counted. Mean±SD of three independent experiments.

FIG. 9A: 1-Mb aCGH identifies KIAA0495 as a novel candidate 1p36.3 TSG. aCGH results of HNC (HONE1) and ESCC (HKESC1) cell lines show that KIAA0495 is located in a heterozygous deletion within 1p36.3. Immortalized normal nasopharyngeal cell line NP69 is used as a control. Each dot represents a single BAC clone. Several validated 1p36 TSGs in the 1p36.3 TSG island are also shown. FIG. 9B: Sequence homology comparison of KIAA0495 and TP73 using the NCBI nucleotide BLAST. Full-length mRNA sequences of KIAA0495 (NR 033711) and TP73 (NM 005427) are compared, and summarized by description and graphics.

FIG. 10A-FIG. 10C. Downregulated expression of KIAA0495 in multiple cancers. FIG. 10A: Boxplot for KIAA0495 expression profiles across multiple cancers [GPL570 platform (HG-U133_Plus_2)] in the GENT2 database (website: gent2.appex.kr/gent2/). T, tumor; N, normal. FIG. 10B: SAGE tag expression analysis in CGAP database (Cancer Genome Anatomy Project; website: cgap.nci.nih.gov/) across multiple tumor/normal tissue libraries. FIG. 10C: Downregulation of KIAA0495 in different stages of colon adenocarcinoma (COAD) and breast invasive carcinoma (BRCA) from the TCGA database by UALCAN portal analysis. Statistical significance between different groups is shown in the table.

FIG. 11A-FIG. 11C. Low KIAA0495 expression is associated with poor survival of multiple cancers. FIG. 11A: Low KIAA0495 expression is associated with poor survival of lung and breast cancer patients. Survival curve is plotted by the Kaplan-Meier plotter (website: kmplot.com) using median value of gene expression as a cutoff. FIG. 11B: Survival curves comparing patients with high and low expressions of colorectal cancer samples (GSE17537) are plotted from the PrognoScan database. Overall survival, disease-free survival and disease-specific survival curves, comparing patients with high (red) and low (blue) expression, are plotted from the PrognoScan database using threshold of corrected p-value<0.05. FIG. 11C: Lower expression of KIAA0495 is associated with poorer survival in bladder and ovarian cancer patients, as demonstrated by Kaplan-Meier survival analysis using SPSS 18 package. Log Rank p-value was calculated.

FIG. 12A: Alignment of 50495 protein sequences from different mammalian species. Asterisk indicates conserved residue. FIG. 12B: KIAA0495 is expressed and unmethylated cell lines of lung, liver (HCC), renal (RCC), ovarian and prostate cancers (except for few cases with both methylated and unmethylated promoter alleles), but commonly silenced and methylated in cervical and bladder cancers as well as lymphoma cell lines. M, methylated; U, unmethylated; Ca, Carcinoma; NKTCL, NK/T-cell lymphoma. FIG. 12C: Downregulation of KIAA0495 further confirmed in representative tumor cell lines by quantitative real-time PCR. Gene expression level in each sample was shown as relative to internal control GAPDH. Normal adult tissues are underlined.

FIG. 13A: High-resolution bisulfite genomic sequencing (BGS) analysis of the KIAA0495 promoter in multiple carcinoma cell lines. Filled circle, methylated; Open circle, unmethylated. MSP primer sites (m1/m2) were indicated. A+T, Aza+TSA treatment. FIG. 13B: KIAA0495 methylation in tumor samples detected by MSP and BGS. KIAA0495 methylation in paired ESCC tissues and normal esophageal tissues examined by MSP. FIG. 13C: BGS analysis of KIAA0495 methylation in representative primary nasopharyngeal carcinoma (NPC) tumors. Filled circle, methylated; Open circle, unmethylated. MSP primer sites (m1/m2) were indicated. FIG. 13D: KIAA0495 methylation in nose swab samples from NPC patients examined by MSP. M, methylated; U, unmethylated.

FIG. 14A: Monolayer-culture colony formation assay of MB231 tumor cells. Quantitative analysis of colony numbers shown as ±SEM of three independent experiments using Student's t-test. *, p<0.05. Expression of SP0495 in MB231 tumor cells detected by Western blot. FIG. 14B: DAPI staining shows SP0495-expressing cells with membrane blebbing and apoptotic bodies. Cells were transfected with pIRES2-ZsGreen1-SP0495 or control vector. Representative images are shown. Red arrows indicated nuclei showing apoptosis-associated changes. FIG. 14C: SP0495 suppresses tumor formation in vivo. The volume of xenograft tumors is monitored at indicated time points for xenograft tumor formation of MB231 cells expressing SP0495 or vector only. Cells were injected subcutaneously into nude mice. Horizontal bars show mean tumor volume and tumor weight. Statistically significant decrease in tumor weight and growth (**, p<0.01; *, p<0.05) upon ectopic SP0495 expression was observed. p values determined by unbiased Student's t-test.

FIG. 15A: SP0495 represses TOPFlash reporter activity in a dosage-dependent manner, while does not affect control FOPFlash activity. **p<0.01. FIG. 15B: Establishment of inducible SP0495 expression T-REx-293 cell line. Immunoblot of SP0495 protein expression in T-REx-293 cells induced with 1 g/mL doxycycline (dox) for 48 h. FIG. 15C: The Top10 KEGG pathways enriched for differentially expressed genes in inducible SP0495 expression T-REx-293 cells, compared with controls.

### DEFINITIONS

The term “SP0495 (small protein of KIAA0495),” as used herein, refers to the protein encoded by the open reading frame 2 (ORF2) of the KIAA0495 (also known as PDAM or TP73-AS1) gene with a chromosomal location of 1p36.3. Depending on the context, “SP0495” may be used to refer to the protein as well as the RNA transcript encoding the protein. The term also encompasses any naturally occurring variants or mutants, interspecies homologs or orthologs, or man-made variants of exemplary human SP0495 protein and coding sequence set forth in SEQ ID NO:1 and SEQ ID NO:2, respectively. A SP0495 protein within the meaning of this application typically has at least 80%, 85%, 90%, 91%, 92%, 93%, 94%, 95%, 96%, 97%, 98%, 99% or higher sequence identity to the human SP0495 protein having the amino acid sequence set forth in SEQ ID NO:1.

In this disclosure the term “or” is generally employed in its sense including “and/or” unless the content clearly dictates otherwise.

As used herein, the term “gene expression” is used to refer to the transcription of a DNA to form an RNA molecule encoding a particular protein (e.g., human SP0495 protein) or the translation of a protein encoded by a polynucleotide sequence. In other words, both mRNA level and protein level encoded by a gene of interest (e.g., ORF2 of KIAA0495 gene) are encompassed by the term “gene expression level” in this disclosure.

In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., serum, plasma, platelets, all blood cells or certain types of blood cells (such as red blood cells), and the like), sputum or saliva, lymph and tongue tissue, cultured cells, e.g., primary cultures, explants, and transformed cells, stool, urine, esophagus biopsy tissue etc. A biological sample is typically obtained from a eukaryotic organism, which may be a mammal, may be a primate and may be a human subject.

In this disclosure the term “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., tongue, colon, prostate, kidney, bladder, lymph node, liver, lung, bone marrow, blood cells, stomach tissue, esophagus, etc.) among other factors. Representative biopsy techniques include, but are not limited to, excisional biopsy, incisional biopsy, needle biopsy, surgical biopsy, and bone marrow biopsy and may comprise colonoscopy or endoscopy. A wide range of biopsy techniques are well known to those skilled in the art who will choose between them and implement them with minimal experimentation.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamine) containing restriction-digested genomic DNA, is not an “isolated” nucleic acid.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in WO01/12654, which may improve the stability (e.g., half-life), bioavailability, and other characteristics of a polypeptide comprising one or more of such D-amino acids. In some cases, one or more, and potentially all of the amino acids of a therapeutic polypeptide have D-chirality.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a variant SP0495 protein used in the method of this invention (e.g., for treating cancer) has at least 80% sequence identity, preferably 85%, 90%, 91%, 92%, 93, 94%, 95%, 96%, 97%, 98%, 99%, or 100% identity, to a reference sequence, e.g., an exemplary human SP0495 protein having the amino acid sequence of SEQ ID NO:1), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75 to 100 or 200 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “bisulfite” as used herein encompasses all types of bisulfites, such as sodium bisulfite, that are capable of chemically converting a cytosine (C) to a uracil (U) without chemically modifying a methylated cytosine and therefore can be used to differentially modify a DNA sequence based on the methylation status of the DNA.

As used herein, a reagent that “differentially modifies” methylated or non-methylated DNA encompasses any reagent that reacts differentially with methylated and unmethylated DNA in a process through which distinguishable products or quantitatively distinguishable results (e.g. degree of binding or precipitation) are generated from methylated and non-methylated DNA, thereby allowing the identification of the DNA methylation status. Such processes may include, but are not limited to, chemical reactions (such as an unmethylated C→U conversion by bisulfite), enzymatic treatment (such as cleavage by a methylation-dependent endonuclease), binding, and precipitation. Thus, an enzyme that preferentially cleaves methylated DNA is one capable of cleaving a DNA molecule at a much higher efficiency when the DNA is methylated, whereas an enzyme that preferentially cleaves unmethylated DNA exhibits a significantly higher efficiency when the DNA is not methylated. In the context of the present invention, a reagent that “differentially modifies” methylated and unmethylated DNA also refers to any reagent that exhibits differential ability in its binding to DNA sequences or precipitation of DNA sequences depending on their methylation status. One class of such reagents consists of methylated DNA binding proteins.

A “CpG-containing genomic sequence” as used herein refers to a segment of DNA sequence at a defined location in the genome of an individual. Typically, a “CpG-containing genomic sequence” is at least 15 contiguous nucleotides in length and contains at least one CpG pair. In some cases, it can be at least 18, 20, 25, 30, 50, 80, 100, 150, 200, 250, or 300 contiguous nucleotides in length and contains at least 2, 3, 4, 5, 6, 7, 8, 9, 10, 15, 20, 25, 30, or more CpG pairs. For any one “CpG-containing genomic sequence” at a given location, e.g., within a region of the human KIAA0495 genomic sequence (such as the region containing the promoter and exon 1), nucleotide sequence variations may exist from individual to individual and from allele to allele even for the same individual. Furthermore, a “CpG-containing genomic sequence” may encompass a nucleotide sequence transcribed or not transcribed for protein production, and the nucleotide sequence can be a protein-coding sequence, a non protein-coding sequence (such as a transcription promoter), or a combination thereof.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used in this application, an “increase” or a “decrease” refers to a detectable positive or negative change in quantity from a comparison control, e.g., an established standard control (such as an average expression level of SP0495 mRNA or SP0495 protein found in healthy, non-cancerous tissue). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within +10% of the standard control, or within +5%, 2%, or even less variation from the standard control.

A “polynucleotide hybridization method” as used herein refers to a method for detecting the presence and/or quantity of a pre-determined polynucleotide sequence based on its ability to form Watson-Crick base-pairing, under appropriate hybridization conditions, with a polynucleotide probe of a known sequence. Examples of such hybridization methods include Southern blot, Northern blot, and in situ hybridization.

“Primers” as used herein refer to oligonucleotides that can be used in an amplification method, such as a polymerase chain reaction (PCR), to amplify a nucleotide sequence based on the polynucleotide sequence corresponding to a gene of interest, e.g., the cDNA or genomic sequence for human KIAA0495 or a portion thereof. Typically at least one of the PCR primers for amplification of a polynucleotide sequence is sequence-specific for that polynucleotide sequence. The exact length of the primer will depend upon many factors, including temperature, source of the primer, and the method used. For example, for diagnostic and prognostic applications, depending on the complexity of the target sequence, the oligonucleotide primer typically contains at least 10, or 15, or 20, or 25 or more nucleotides, although it may contain fewer nucleotides or more nucleotides. The factors involved in determining the appropriate length of primer are readily known to one of ordinary skill in the art. The primers used in particular embodiments are shown in Table 7 of the disclosure where their specific applications are indicated. In this disclosure the term “primer pair” means a pair of primers that hybridize to opposite strands a target DNA molecule or to regions of the target DNA which flank a nucleotide sequence to be amplified. In this disclosure, the term “primer site” means the area of the target DNA or other nucleic acid to which a primer hybridizes.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is attached to a probe or a molecule with defined binding characteristics (e.g., a polypeptide with a known binding specificity or a polynucleotide), so as to allow the presence of the probe (and therefore its binding target) to be readily detectable.

“Standard control” as used herein refers to a predetermined amount or concentration of a polynucleotide sequence or polypeptide, e.g., SP0495 mRNA or SP0495 protein, that is present in an established normal cancer-free tissue sample, e.g., a normal esophagus epithelial tissue sample. The standard control value is suitable for the use of a method of the present invention, to serve as a basis for comparing the amount of SP0495 mRNA or SP0495 protein that is present in a test sample. An established sample serving as a standard control provides an average amount of SP0495 mRNA or SP0495 protein that is typical for a specific tissue sample (e.g., esophagus epithelial tissue) of an average, healthy human without any neoplastic disease especially cancer as conventionally defined at the specific anatomic site. A standard control value may vary depending on the nature/origin of the sample, sample processing and detection method, as well as other factors such as the gender, age, ethnicity of the subjects based on whom such a control value is established.

The term “average,” as used in the context of describing a human who is healthy, free of any cancer (especially at a specified anatomic site) as conventionally defined, refers to certain characteristics, especially the amount of human SP0495 mRNA or SP0495 protein, found in the person's pertinent tissue, that are representative of a randomly selected group of healthy humans who are free of any cancer. This selected group should comprise a sufficient number of humans such that the average amount of SP0495 mRNA or protein in the relevant tissue type among these individuals reflects, with reasonable accuracy, the corresponding amount of SP0495 mRNA or protein in the general population of healthy humans. In addition, the selected group of humans generally have a similar age to that of a subject whose pertinent tissue sample is tested for potential indication of cancer. Moreover, other factors such as gender, ethnicity, medical history are also considered and preferably closely matching between the profiles of the test subject and the selected group of individuals establishing the “average” value.

The term “amount” as used in this application refers to the quantity of a polynucleotide of interest or a polypeptide of interest, e.g., human SP0495 mRNA or SP0495 protein, present in a sample. Such quantity may be expressed in the absolute terms, i.e., the total quantity of the polynucleotide or polypeptide in the sample, or in the relative terms, i.e., the concentration of the polynucleotide or polypeptide in the sample.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of an polynucleotide encoding SP0495 mRNA is the amount of said polynucleotide to achieve an increased level of SP0495 protein expression or biological activity, such that the symptoms of cancer are reduced, reversed, eliminated, prevented, or delayed of the onset in a patient who has been given the polynucleotide for therapeutic purposes. An amount adequate to accomplish this is defined as the “therapeutically effective dose.” The dosing range varies with the nature of the therapeutic agent being administered and other factors such as the route of administration and the severity of a patient's condition.

The term “subject” or “subject in need of treatment,” as used herein, includes individuals who seek medical attention due to risk of cancer or actual suffering from cancer. Subjects also include individuals currently undergoing therapy that seek manipulation of the therapeutic regimen. Subjects or individuals in need of treatment include those that demonstrate symptoms of cancer or are at risk of suffering from cancer or its symptoms. For example, a subject in need of treatment includes individuals with a genetic predisposition or family history for cancer (e.g., breast, colorectal, or esophageal cancer), those that have suffered relevant symptoms in the past, those that have been exposed to a triggering substance or event, as well as those suffering from chronic or acute symptoms of the condition. A “subject in need of treatment” may be at any age of life.

“Inhibitors,” “activators,” and “modulators” of SP0495 protein are used to refer to inhibitory, activating, or modulating molecules, respectively, identified using in vitro and in vivo assays for SP0495 protein binding or signaling, e.g., ligands, agonists, antagonists, and their homologs and mimetics. The term “modulator” includes inhibitors and activators. Inhibitors are agents that, e.g., partially or totally block carbohydrate binding, decrease, prevent, delay activation, inactivate, desensitize, or down-regulate the activity of SP0495 protein. In some cases, the inhibitor directly or indirectly binds to SP0495 protein, such as a neutralizing antibody. Inhibitors, as used herein, are synonymous with inactivators and antagonists. Activators are agents that, e.g., stimulate, increase, facilitate, enhance activation, sensitize or up-regulate the activity of SP0495 protein. Modulators include SP0495 protein ligands or binding partners, including modifications of naturally-occurring ligands and synthetically-designed ligands, antibodies and antibody fragments, antagonists, agonists, small molecules including carbohydrate-containing molecules, siRNAs, RNA aptamers, and the like.

### DETAILED DESCRIPTION OF THE INVENTION

### I. Introduction

Despite the rapid advancement in medical sciences and steady improvement in cancer therapy, cancer remains a significant health concern with grave implications in both developed countries as well as in developing countries. Cancer patients who receive the diagnosis at later stages of the disease often face a grim prognosis, since therapeutic options and effectiveness diminish as the disease progresses further along. Early detection of cancer is therefore critical for improving patient survival rate. Moreover, it is also of practical importance to predict the likelihood of mortality from cancer among patients who have already received a cancer diagnosis for any time period after the diagnosis.

1p36 is one of the more frequently deleted chromosome regions in a variety of cancers. Various genetic studies have been carried out to identify candidate tumor suppressor genes (TSG) at this locus. The present inventors have now discovered that the KIAA0495 gene, located at 1p36.3 and previously thought as encoding a long non-coding RNA (lncRNA) only, in fact encodes by its open reading frame 2 (ORF2) a small protein termed SP0495, and that this protein is a tumor suppressor silenced in many cancer types via hypermethylation of the promoter region of the KIAA0495 gene. It has been further illustrated that the downregulation of SP0495 is correlated with poor survival among cancer patients and that restoration of SP0495 expression in the cancer cells can induce apoptosis and cell cycle arrest in the cancer cells, promote autophagy, and inhibit cancer growth in vitro and in vivo. This invention provides a method to specifically detect promoter CpG methylation of the promoter region of the KIAA0495 gene in cancers, and its methylation serving as a biomarker for early detection of cancer. Methylation-specific PCR (MSP) primers for KIAA0495 promoter sequence are tested for not amplifying any not-bisulfited DNA, confirming the detection specificity of KIAA0495 promoter methylation in this invention. SP0495 downregulation/silencing by promoter methylation is detected in various cancer cell lines and primary cancers, but not in immortalized non-cancerous cells or normal tissues. In addition, the present invention provides a method for suppressing cancer cell proliferation by restoring SP0495 expression in SP0495-silenced cancer cells. The invention also provides a detection method for cancer, a prognosis method for cancer mortality, and a detection kit useful for such a method.

### II. General Methodology

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

The sequence of interest used in this invention, e.g., the polynucleotide sequence of the human KIAA0495 gene, and synthetic oligonucleotides (e.g., primers) useful for amplifying the coding sequence for the SP0495 protein can be verified using, e.g., the chain termination method for sequencing double-stranded templates of Wallace et al., Gene 16: 21-26 (1981).

### III. Acquisition of Tissue Samples and Analysis of SP0495 mRNA or Genomic DNA

The present invention relates to measuring the amount of SP0495 mRNA or analyzing the methylation pattern of KIAA0495 genomic DNA found in a person's tissue sample as a means to detect the presence, to assess the risk of developing, and/or to monitor the progression or treatment efficacy of a cancer affecting the tissue type. Thus, the first steps of practicing this invention are to obtain an appropriate tissue sample from a test subject and extract mRNA or DNA from the sample.

### A. Acquisition and Preparation of Tissue Samples

A tissue sample of the appropriate type for the kind of cancer being assessed is obtained from a person to be tested or monitored for the cancer using a method of the present invention. Collection of the tissue sample from an individual is performed in accordance with the standard protocol hospitals or clinics generally follow, such as during a biopsy or a blood draw. An appropriate amount of tissue is collected and may be stored according to standard procedures prior to further preparation.

The analysis of SP0495 mRNA or DNA found in a patient's tissue according to the present invention may be performed using a sample obtained by routine procedures, e.g., biopsy or blood draw. The methods for preparing tissue samples for nucleic acid extraction are well known among those of skill in the art. For example, a subject's tissue or blood sample should be first treated to disrupt cellular membrane so as to release nucleic acids contained within the cells.

### B. Extraction and Quantitation of RNA

There are numerous methods for extracting mRNA from a biological sample. The general methods of mRNA preparation (e.g., described by Sambrook and Russell, Molecular Cloning: A Laboratory Manual 3d ed., 2001) can be followed; various commercially available reagents or kits, such as Trizol reagent (Invitrogen, Carlsbad, CA), Oligotex Direct mRNA Kits (Qiagen, Valencia, CA), RNeasy Mini Kits (Qiagen, Hilden, Germany), and PolyATtract® Series 9600™ (Promega, Madison, WI), may also be used to obtain mRNA from a biological sample from a test subject. Combinations of more than one of these methods may also be used.

It is essential that all contaminating DNA be eliminated from the RNA preparations. Thus, careful handling of the samples, thorough treatment with DNase, and proper negative controls in the amplification and quantification steps should be used.

1. PCR-Based Quantitative Determination of mRNA Level

Once mRNA is extracted from a sample, the amount of human SP0495 mRNA may be quantified. The preferred method for determining the mRNA level is an amplification-based method, e.g., by polymerase chain reaction (PCR), especially reverse transcription-polymerase chain reaction (RT-PCR).

Prior to the amplification step, a DNA copy (cDNA) of the human SP0495 mRNA must be synthesized. This is achieved by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, MN, 1993; Egger et al., J. Clin. Microbiol. 33:1442-1447, 1995; and U.S. Pat. No. 5,075,212.

The general methods of PCR are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled through a denaturing region, a primer annealing region, and an extension reaction region automatically. Machines specifically adapted for this purpose are commercially available.

Although PCR amplification of the target mRNA is typically used in practicing the present invention. One of skill in the art will recognize, however, that amplification of a mRNA species in a sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to quantitatively determining the amount of mRNA species in a sample. For a review of branched-DNA signal amplification for direct quantitation of nucleic acid sequences in clinical samples, see Nolte, Adv. Clin. Chem. 33:201-235, 1998.

### 2. Other Quantitative Methods

The SP0495 mRNA can also be detected using other standard techniques, well known to those of skill in the art. Although the detection step is typically preceded by an amplification step, amplification is not required in the methods of the invention. For instance, the mRNA may be identified by size fractionation (e.g., gel electrophoresis), whether or not proceeded by an amplification step. After running a sample in an agarose or polyacrylamide gel and labeling with ethidium bromide according to well-known techniques (see, e.g., Sambrook and Russell, supra), the presence of a band of the same size as the standard comparison is an indication of the presence of a target mRNA, the amount of which may then be compared to the control based on the intensity of the band. Alternatively, oligonucleotide probes specific to SP0495 mRNA can be used to detect the presence of such mRNA species and indicate the amount of mRNA in comparison to the standard comparison, based on the intensity of signal imparted by the probe.

Sequence-specific probe hybridization is a well-known method of detecting a particular nucleic acid comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch.

A number of hybridization formats well known in the art, including but not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

The hybridization complexes are detected according to well-known techniques. Nucleic acid probes capable of specifically hybridizing to a target nucleic acid, i.e., the mRNA or the amplified DNA, can be labeled by any one of several methods typically used to detect the presence of hybridized nucleic acids. One common method of detection is the use of autoradiography using probes labeled with ³H, ¹²⁵I, ³⁵S, ¹⁴C, or ³²P, or the like. The choice of radioactive isotope depends on research preferences due to ease of synthesis, stability, and half lives of the selected isotopes. Other labels include compounds (e.g., biotin and digoxigenin), which bind to anti-ligands or antibodies labeled with fluorophores, chemiluminescent agents, and enzymes. Alternatively, probes can be conjugated directly with labels such as fluorophores, chemiluminescent agents or enzymes. The choice of label depends on sensitivity required, ease of conjugation with the probe, stability requirements, and available instrumentation.

The probes and primers necessary for practicing the present invention can be synthesized and labeled using well known techniques. Oligonucleotides used as probes and primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides is by either native acrylamide gel electrophoresis or by anion-exchange HPLC as described in Pearson and Regnier, J. Chrom., 255:137-149, 1983.

### C. Detection of Methylation in KIAA0495 Genomic Sequence

Methylation status of a segment of KIAA0495 genomic sequence containing one or more CpG (cytosine-guanine dinucleotide) pairs is investigated to provide indication as to whether a test subject is suffering from cancer, whether the subject is at risk of developing cancer, or whether the subject's cancer is worsening or improving, including assessing the relative mortality from cancer among patients who have been diagnosed with cancer.

Typically a segment of the KIAA0495 genomic sequence that includes the 5′ untranslated region (such as the promoter region) and includes one or more CpG nucleotide pairs, optionally 20 or more CpGs, is analyzed for methylation pattern. For example, SEQ ID NO:3 or a portion thereof comprising at least 10, possibly 15, 20, 25, or 30 or more CpG dinucleotide pairs, can be used to determine how many of the CpG pairs within the sequence are methylated and how many are not methylated. The sequence being analyzed should be long enough to contain at least 1 CpG dinucleotide pair and detection of methylation at this CpG site is typically adequate indication of the presence of cancer cells. The length of the sequence being analyzed is usually at least 15 or 20 contiguous nucleotides, and may be longer with at least 25, 30, 50, 100, 200, 300, 400, or more contiguous nucleotides. At least one, typically 2 or more, often 3, 4, 5, 6, 7, 8, 9, 10, 15, 20, 25, 30 or more, CpG nucleotide pairs are present within the sequence. In some cases where multiple (2 or more) CpG sites are analyzed for methylation status, when at least 50% of the CpG pairs within the analyzed genomic sequence are shown to be methylated, subject being tested is deemed to have cancer, especially the cancer affecting the same tissue type. For example, SEQ ID NO:3, a segment of KIAA0495 genomic sequence, is such a CpG-containing genomic sequence useful for the analysis. Some or majority of the CpG pairs in this region are found to be methylated in established cancer cell lines and samples taken from cancerous tissues, whereas non-cancerous corresponding tissues and cells shows very few, if any at all, methylated CpG sites. For the purpose of determining the methylation pattern of a KIAA0495 genomic sequence, bisulfite treatment followed by DNA sequencing is particularly useful, since bisulfite converts an unmethylated cytosine (C) to a uracil (U) while leaving methylated cytosines unchanged, allowing immediate identification through a DNA sequencing process. Optionally, an amplification process such as PCR is included after the bisulfite conversion and before the DNA sequencing.

### 1. DNA Extraction and Treatment

Methods for extracting DNA from a biological sample are well known and routinely practiced in the art of molecular biology, see, e.g., Sambrook and Russell, supra. RNA contamination should be eliminated to avoid interference with DNA analysis. The DNA is then treated with a reagent capable of modifying DNA in a methylation differential manner, i.e., different and distinguishable chemical structures will result from a methylated cytosine (C) residue and an unmethylated C residue following the treatment. Typically, such a reagent reacts with the unmethylated C residue(s) in a DNA molecule and converts each unmethylated C residue to a uracil (U) residue, whereas the methylated C residues remain unchanged. This unmethylated C→U conversion allows detection and comparison of methylation status based on changes in the primary sequence of the nucleic acid. An exemplary reagent suitable for this purpose is bisulfite, such as sodium bisulfite. Methods for using bisulfite for chemical modification of DNA are well known in the art (see, e.g., Herman et al., Proc. Natl. Acad. Sci. USA 93:9821-9826, 1996).

As a skilled artisan will recognize, any other reagents that are unnamed here but have the same property of chemically (or through any other mechanism) modifying methylated and unmethylated DNA differentially can be used for practicing the present invention. For instance, methylation-specific modification of DNA may also be accomplished by methylation-sensitive restriction enzymes, some of which typically cleave an unmethylated DNA fragment but not a methylated DNA fragment, while others (e.g., methylation-dependent endonuclease McrBC) cleave DNA containing methylated cytosines but not unmethylated DNA. In addition, a combination of chemical modification and restriction enzyme treatment, e.g., combined bisulfite restriction analysis (COBRA) (Xiong et al. 1997 Nucleic Acids Res. 25(12): 2532-2534), is useful for practicing the present invention. Other available methods for detecting DNA methylation include, for example, methylation-sensitive restriction endonucleases (MSREs) assay by either Southern blot or PCR analysis, methylation specific or methylation sensitive-PCR (MS-PCR), methylation-sensitive single nucleotide primer extension (Ms-SnuPE), high resolution melting (HRM) analysis, bisulifte sequencing, pyrosequencing, methylation-specific single-strand conformation analysis (MS-SSCA), methylation-specific denaturing gradient gel electrophoresis (MS-DGGE), methylation-specific melting curve analysis (MS-MCA), methylation-specific denaturing high-performance liquid chromatography (MS-DHPLC), methylation-specific microarray (MSO). These assays can be either PCR analysis, quantitative analysis with fluorescence labelling or Southern blot analysis. Exemplary methylation sensitive DNA cleaving reagent such as restriction enzymes include AatII, AciI, AclI, AgeI, AscI, Asp718, AvaI, BbrPl, BceAI, BmgBI, BsaAI, BsaHI, BsiEI, BsiWI, BsmBI, BspDI, BsrFI, BssHII, BstBI, BstUI, ClaI, EagI, EagI-HF™, FauI, FseI, FspI, HaeII, HgaI, HhaI, HinP1I, HpaII, Hpy99I, HpyCH4IV, KasI, MluI, NarI, NgoMIV, NotI, NotI-HF™, NruI, Nt.BsmAI, PaeR7I, PspXI, PvuI, RsrII, SacII, SalI, SalI-HF™, SfoI, SgrAI, SmaI, SnaBI or TspMI.

### 2. Optional Amplification and Sequence Analysis

Following the modification of DNA in a methylation-differential manner, the treated DNA is then subjected to sequence-based analysis, such that the methylation status of the KIAA0495 genomic sequence may be determined. An amplification reaction is optional prior to the sequence analysis after methylation specific modification. A variety of polynucleotide amplification methods are well established and frequently used in research. For instance, the general methods of polymerase chain reaction (PCR) for polynucleotide sequence amplification are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

Although PCR amplification is typically used in practicing the present invention, one of skill in the art will recognize that amplification of the relevant genomic sequence may be accomplished by any known method, such as the ligase chain reaction (LCR), transcription-mediated amplification, and self-sustained sequence replication or nucleic acid sequence-based amplification (NASBA), each of which provides sufficient amplification.

Techniques for polynucleotide sequence determination are also well established and widely practiced in the relevant research field. For instance, the basic principles and general techniques for polynucleotide sequencing are described in various research reports and treatises on molecular biology and recombinant genetics, such as Wallace et al., supra; Sambrook and Russell, supra, and Ausubel et al., supra. DNA sequencing methods routinely practiced in research laboratories, either manual or automated, can be used for practicing the present invention. Additional means suitable for detecting changes (e.g., C→U) in a polynucleotide sequence for practicing the methods of the present invention include but are not limited to mass spectrometry, primer extension, polynucleotide hybridization, real-time PCR, melting curve analysis, high resolution melting analysis, heteroduplex analysis, pyrosequencing, and electrophoresis.

### IV. Quantitation of Polypeptides

### A. Obtaining Samples

The first step of practicing the present invention is to obtain a sample of the appropriate tissue type from a subject being tested, assessed, or monitored for cancer, the risk of developing cancer, or the severity/progression/mortality prospect of the cancer. Samples of the same type should be taken from both a control group (normal individuals not suffering from any neoplasia affecting the same tissue type) and a test group (subjects being tested for possible cancer of the relevant type). Standard procedures routinely employed in hospitals or clinics are typically followed for this purpose, as stated in the previous section.

For the purpose of detecting the presence of cancer or assessing the risk of developing cancer in test subjects, individual patients' tissue samples of the corresponding type may be taken and the level of human SP0495 protein may be measured and then compared to a standard control. If a decrease in the level of human SP0495 protein is observed when compared to the control level, the test subject is deemed to have cancer or have an elevated risk of developing cancer affecting the tissue type. For the purpose of monitoring disease progression or assessing therapeutic effectiveness in cancer patients, individual patient's relevant tissue samples may be taken at different time points, such that the level of human SP0495 protein can be measured to provide information indicating the state of disease. For instance, when a patient's SP0495 protein level shows a general trend of increase over time, the patient is deemed to be improving in the severity of cancer or the therapy the patient has been receiving is deemed effective. A lack of change in a patient's SP0495 protein level or a continuing trend of decrease on other hand would indicate a worsening of the condition and ineffectiveness of the therapy given to the patient. Generally, a lower SP0495 protein level seen in a patient indicates a more severe form of the cancer the patient is suffering from and a worse prognosis of the disease, as manifested in shorter life expectancy, higher rate of metastasis, resistance to therapy etc. Among cancer patients, one who has a lower level of SP0495 protein expression in the cancer tissue sample than that found in the same type of cancer tissue sample from a second cancer patient has a higher likelihood of mortality compared to the second patient for any defined time period, such as 1-5 years post-diagnosis of the cancer.

### B. Preparing Samples for SP0495 Protein Detection

The tissue sample from a subject is suitable for the present invention and can be obtained by well-known methods and as described in the previous section. In certain applications of this invention, blood samples or epithelial tissue or lining may be the preferred sample type.

### C. Determining the Level of Human SP0495 Protein

A protein of any particular identity, such as SP0495 protein, can be detected using a variety of immunological assays. In some embodiments, a sandwich assay can be performed by capturing the polypeptide from a test sample with an antibody having specific binding affinity for the polypeptide. The polypeptide then can be detected with a labeled antibody having specific binding affinity for it. Such immunological assays can be carried out using microfluidic devices such as microarray protein chips. A protein of interest (e.g., human SP0495 protein) can also be detected by gel electrophoresis (such as 2-dimensional gel electrophoresis) and western blot analysis using specific antibodies. Alternatively, standard immunohistochemical techniques can be used to detect a given protein (e.g., human SP0495 protein), using the appropriate antibodies. Both monoclonal and polyclonal antibodies (including antibody fragment with desired binding specificity) can be used for specific detection of the polypeptide. Such antibodies and their binding fragments with specific binding affinity to a particular protein (e.g., human SP0495 protein) can be generated by known techniques.

Other methods may also be employed for measuring the level of SP0495 protein in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

### V. Establishing a Standard Control

In order to establish a standard control for practicing the method of this invention, a group of healthy persons free of any neoplastic disease (especially any form of cancer) as conventionally defined is first selected. These individuals are within the appropriate parameters, if applicable, for the purpose of screening for and/or monitoring cancer using the methods of the present invention. Optionally, the individuals are of same gender, similar age, or similar ethnic background.

The healthy status of the selected individuals is confirmed by well-established, routinely employed methods including but not limited to general physical examination of the individuals and general review of their medical history.

Furthermore, the selected group of healthy individuals must be of a reasonable size, such that the average amount/concentration of human SP0495 mRNA or SP0495 protein in the tissue sample obtained from the group can be reasonably regarded as representative of the normal or average level in this tissue type among the general population of healthy people. Preferably, the selected group comprises at least 10, 20, 50, 100 or more human subjects.

Once an average value for the SP0495 mRNA or protein is established based on the individual values found in each subject of the selected healthy control group, this average or median or representative value or profile is considered a standard control. A standard deviation is also determined during the same process. In some cases, separate standard controls may be established for separately defined groups having distinct characteristics such as age, gender, or ethnic background.

### VI. Treatment of Cancer

By illustrating the correlation of suppressed expression of SP0495 protein and cancers such as colorectal, breast, and esophageal cancer, the present invention further provides a means for treating patients suffering from the cancer or at heightened risk of developing the cancer at a later time: by way of increasing SP0495 protein expression or biological activity. As used herein, treatment of cancer encompasses reducing, reversing, lessening, or eliminating one or more of the symptoms of the cancer, as well as preventing or delaying the onset of one or more of the relevant symptoms. Additionally, since certain risk factors for any particular cancer are well known, preventive measures can be prescribed to patients at risk of developing the cancer such as reducing or eliminating alcohol and tobacco consumption and adopting a healthy diet. For individuals who have been deemed to have an increased risk of developing cancer by the method of this invention and who are then diagnosed as actually having already developed cancer (e.g., by conventional diagnostic methods such as X-ray and/or CT scan of the affected area in addition to pathological assessment), various treatment strategies are available for treating cancer in these patients including, but not limited to, surgery, chemotherapy, radiotherapy, immunotherapy, photodynamic therapy, or any combination thereof.

### A. Increasing SP0495 Expression or Activity

### 1. Nucleic Acids Encoding SP0495 Proteins

Enhancement of SP0495 expression can be achieved through the use of nucleic acids encoding a functional SP0495 protein. Such nucleic acids can be single-stranded nucleic acids (such as mRNA) or double-stranded nucleic acids (such as DNA) that can translate into an active form of SP0495 protein under favorable conditions.

In one embodiment, the SP0495-encoding nucleic acid is provided in the form of an expression cassette, typically recombinantly produced, having a promoter operably linked to the polynucleotide sequence encoding the SP0495 protein. In some cases, the promoter is a universal promoter that directs gene expression in all or most tissue types; in other cases, the promoter is one that directs gene expression specifically in tissues and cells relevant to or involved in a particular cancer. Administration of such nucleic acids can increase the SP0495 protein expression in the target tissue or cell type. Since the human SP0495 coding sequence is provided herein as SEQ ID NO:2, and its amino acid sequence is provided herein as SEQ ID NO:1, one can derive a suitable SP0495-encoding nucleic acid from the sequence, species homologs, and variants of these sequences, one can derive a suitable SP0495-encoding nucleic acid from the sequence, species homologs, and variants of these sequences.

### 2. SP0495 Protein

By directly administering an effective amount of an active SP0495 protein to a patient suffering from cancer and exhibiting suppressed SP0495 protein expression or activity, the disease may also be effectively treated. For example, this can be achieved by administering a recombinantly produced SP0495 protein possessing its biological activity to the patient suffering from cancer. Formulations and methods for delivering a protein- or polypeptide-based therapeutic agent are well known in the art.

### 3. Activators of SP0495 Protein

Increased SP0495 protein activity can be achieved with an agent that is capable of activating the expression of SP0495 protein or enhancing the activity of SP0495 protein. For example, a demethylating agent (e.g., 5-Aza) may be able to activate KIAA0495 gene expression by removing the suppression of SP0495 gene expression caused by methylation of the promoter region of this gene. Other activating agents may include transcriptional activators specific for the KIAA0495 promoter and/or enhancer. Such activating agents can be screened for and identified using the SP0495 expression assays described in the examples herein.

Agonists of the SP0495 protein, such as an activating antibody, are another kind of activators of the SP0495 protein. Such activators act by enhancing the biological activity of the SP0495 protein, typically (but not necessarily) by direct binding with the SP0495 protein and/or its interacting proteins. Preliminary screening for such agonists may start with a binding assay for identifying molecules that physically interact with SP0495 protein.

### B. Pharmaceutical Compositions

### 1. Formulations

Compounds of the present invention are useful in the manufacture of a pharmaceutical composition or a medicament. A pharmaceutical composition or medicament can be administered to a subject for the treatment of various types of cancer, where the expression of SP0495 is suppressed.

Compounds used in the present invention, e.g., a SP0495 protein, a nucleic acid encoding a SP0495 protein, or an activator of SP0495 expression, are useful in the manufacture of a pharmaceutical composition or a medicament comprising an effective amount thereof in conjunction or mixture with excipients or carriers suitable for application.

An exemplary pharmaceutical composition for enhancing SP0495 expression comprises (i) an express cassette comprising a polynucleotide sequence encoding a human SP0495 protein as described herein, and (ii) a pharmaceutically acceptable excipient or carrier. The terms pharmaceutically-acceptable and physiologically-acceptable are used synonymously herein. The expression cassette may be provided in a therapeutically effective dose for use in a method for treatment as described herein.

A SP0495 protein or a nucleic acid encoding a SP0495 protein can be administered via liposomes, which serve to target the conjugates to a particular tissue or cell type, as well as increase the half-life of the composition. Liposomes include emulsions, foams, micelles, insoluble monolayers, liquid crystals, phospholipid dispersions, lamellar layers and the like. In these preparations the inhibitor to be delivered is incorporated as part of a liposome, alone or in conjunction with a molecule which binds to, e.g., a receptor prevalent among the targeted cells or tissues relevant to the specific type of cancer, or with other therapeutic or immunogenic compositions. Thus, liposomes filled with a desired active agent of the invention can be directed to the site of treatment, where the liposomes then deliver the therapeutic compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) Ann. Rev. Biophys. Bioeng. 9: 467, U.S. Pat. Nos. 4,235,871, 4,501,728 and 4,837,028.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, parenterally, or rectally.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

### 2. Routes of Administration

Suitable formulations for topical application, e.g., to the skin and eyes, are preferably aqueous solutions, ointments, creams or gels well known in the art. Such may contain solubilizers, stabilizers, tonicity enhancing agents, buffers and preservatives.

Suitable formulations for transdermal application include an effective amount of a compound or agent of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a SP0495 protein or a nucleic acid encoding a SP0495 protein, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active agent.

Compounds and agents of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the active ingredient, e.g., a SP0495 protein or a nucleic acid encoding a SP0495 protein, may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The active compound or agent of the present invention can also be formulated in rectal compositions, for example, suppositories or retention enemas, for example, containing conventional suppository bases, for example, cocoa butter or other glycerides.

Furthermore, the active ingredient can be formulated as a depot preparation. Such long-acting formulations can be administered by implantation (for example, subcutaneously or intramuscularly) or by intramuscular injection. Thus, for example, the active ingredient can be formulated with suitable polymeric or hydrophobic materials (for example as an emulsion in an acceptable oil) or ion exchange resins, or as sparingly soluble derivatives, for example, as a sparingly soluble salt.

A pharmaceutical composition or medicament of the present invention comprises (i) an effective amount of a composition as described herein that increases the level or activity of the SP0495 protein, and (ii) another therapeutic agent, e.g., a known anti-cancer therapeutic agent. When used with an active composition or agent of the present invention, such therapeutic agent may be used individually, sequentially, or in combination with one or more other such therapeutic agents (e.g., a first known anti-cancer therapeutic agent, a second known anti-cancer therapeutic agent, and a compound of the present invention). Administration may be by the same or different route of administration separately or together in the same pharmaceutical formulation.

### 3. Dosage

Pharmaceutical compositions or medicaments can be administered to a subject at a therapeutically effective dose to prevent, treat, or control a cancer (where the involved cells or tissues show suppressed SP0495 expression) as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular compound in a particular subject. For example, each type of SP0495 protein or nucleic acid encoding a SP0495 protein will likely have a unique dosage. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies and repetition rates.

To achieve the desired therapeutic effect, compounds or agents may be administered for multiple days at the therapeutically effective daily dose. Thus, therapeutically effective administration of compounds to treat a pertinent condition or disease described herein in a subject requires periodic (e.g., daily) administration that continues for a period ranging from three days to two weeks or longer. Typically, the active agents will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

Optimum dosages, toxicity, and therapeutic efficacy of such compounds or agents may vary depending on the relative potency of individual compounds or agents and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any agents used in the methods of the invention, the therapeutically effective dose can be estimated initially from cell culture assays. A dose can be formulated in animal models to achieve a circulating plasma concentration range that includes the IC₅₀ (the concentration of the agent that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of agents is from about 1 ng/kg to 100 mg/kg for a typical subject.

Exemplary dosages for SP0495 protein or a nucleic acid encoding a SP0495 protein described herein are provided. Dosage for a SP0495-encoding nucleic acid, such as an expression cassette, can be between 0.1-0.5 mg/day, with intravenous administration (e.g., 5-30 ng/kg bodyweight). Small organic compounds activators can be administered orally at between 5-1000 mg, or by intravenous infusion at between 10-500 mg/ml. Antibody (including monoclonal antibody) activators can be administered by intravenous injection or infusion at 50-500 mg/ml (over 120 minutes); 1-500 mg/kg (over 60 minutes); or 1-100 mg/kg (bolus) five times weekly. SP0495 protein or peptide activators can be administered subcutaneously at 10-500 mg; 0.1-500 mg/kg intravenously twice daily, or about 50 mg once weekly, or 25 mg twice weekly.

Pharmaceutical compositions of the present invention can be administered alone or in combination with at least one additional therapeutic compound. Exemplary advantageous therapeutic compounds include systemic and topical anti-inflammatories, pain relievers, anti-histamines, anesthetic compounds, and the like. The additional therapeutic compound can be administered at the same time as, or even in the same composition with, main active ingredient (e.g., a SP0495 protein or a nucleic acid encoding the protein). The additional therapeutic compound can also be administered separately, in a separate composition, or a different dosage form from the main active ingredient. Some doses of the main ingredient, such as a SP0495 protein or a nucleic acid encoding a SP0495 protein, can be administered at the same time as the additional therapeutic compound, while others are administered separately, depending on the particular symptoms and characteristics of the individual.

The dosage of a pharmaceutical composition of the invention can be adjusted throughout treatment, depending on severity of symptoms, frequency of recurrence, and physiological response to the therapeutic regimen. Those of skill in the art commonly engage in such adjustments in therapeutic regimen.

### VII. Kits and Devices

The invention provides compositions and kits for practicing the methods described herein to assess the level of SP0495 mRNA or SP0495 protein in a subject, which can be used for various purposes such as determining the risk of developing cancer, diagnosing cancer, or monitoring cancer progression in a patient, including assessing the likelihood of mortality from cancer.

Kits for carrying out assays for determining SP0495 mRNA level typically include at least one oligonucleotide useful for specific hybridization with at least one segment of the SP0495 coding sequence (i.e., ORF2 of the KIAA0495 gene) or its complementary sequence. Optionally, this oligonucleotide is labeled with a detectable moiety. In some cases, the kits may include at least two oligonucleotide primers that can be used in the amplification of at least one segment of the KIAA0495 ORF2 DNA or SP0495 mRNA by PCR, particularly by RT-PCR.

Kits for carrying out assays for determining SP0495 protein level typically include at least one antibody useful for specific binding to the SP0495 protein amino acid sequence. Optionally, this antibody is labeled with a detectable moiety. The antibody can be either a monoclonal antibody or a polyclonal antibody. In some cases, the kits may include at least two different antibodies, one for specific binding to the SP0495 protein (i.e., the primary antibody) and the other for detection of the primary antibody (i.e., the secondary antibody), which is often attached to a detectable moiety.

Typically, the kits also include an appropriate standard control. The standard controls indicate the average value of SP0495 protein or mRNA in a specific tissue type of healthy subjects not suffering from cancer affecting the corresponding tissue type. In some cases such standard control may be provided in the form of a set value. In addition, the kits of this invention may provide instruction manuals to guide users in analyzing test samples and assessing the presence, risk, or state of cancer corresponding to the tissue type in a test subject.

In a further aspect, the present invention can also be embodied in a device or a system comprising one or more such devices, which is capable of carrying out all or some of the method steps described herein. For instance, in some cases, the device or system performs the following steps upon receiving a particular tissue sample corresponding to the cancer type being assessed, e.g., an esophagus epithelial tissue sample taken from a subject being tested for detecting esophageal cancer, assessing the risk of developing esophageal cancer, or monitored for progression of the condition: (a) determining in sample the amount or concentration of SP0495 mRNA or SP0495 protein; (b) comparing the amount or concentration with a standard control value; and (c) providing an output indicating whether the cancer being assessed, e.g., esophageal cancer, is present in the subject or whether the subject is at risk of developing esophageal cancer, or whether there is a change, i.e., worsening or improvement, in the subject's esophageal cancer condition. In other cases, the device or system of the invention performs the task of steps (b) and (c), after step (a) has been performed and the amount or concentration from (a) has been entered into the device. Preferably, the device or system is partially or fully automated.

### Examples

The following examples are provided by way of illustration only and not by way of limitation. Those of skill in the art will readily recognize a variety of non-critical parameters that could be changed or modified to yield essentially the same or similar results.

### Abstract

Peptides/small proteins, encoded by noncanonical open reading frames (ORF) of previously claimed non-coding RNAs, have recently been recognized possessing important, but largely uncharacterized, biological functions. 1p36 is an important tumor suppressor gene (TSG) locus frequently deleted in multiple cancers, with critical TSGs like TP73, PRDM16 and CHD5 already validated. Our CpG methylome analysis identified a silenced 1p36.3 gene KIAA0495, previously thought coding long non-coding RNA. We found that the open reading frame 2 of KIAA0495 is actually protein-coding and translating, encoding a small protein SP0495. KIAA0495 transcript is broadly expressed in multiple normal tissues, but frequently silenced by promoter CpG methylation in multiple tumor cell lines and primary tumors including colorectal, esophageal and breast cancers. Its downregulation/methylation is associated with poor survival of cancer patients. SP0495 induces tumor cell apoptosis, cell cycle arrest, senescence and autophagy, and inhibits tumor cell growth in vitro and in vivo. Mechanistically, SP0495 binds to phosphoinositides (PtdIns(3)P, PtdIns(3,5)P2) as a lipid-binding protein, inhibits AKT phosphorylation and its downstream signaling, and further represses oncogenic AKT/mTOR, NF-κB and Wnt/β-catenin signaling. SP0495 also regulates the stability of autophagy regulators BECN1 and SQSTM1/p62 through modulating phosphoinositides turnover and autophagic/proteasomal degradation. Thus, we discovered and validated a 1p36.3 small protein SP0495, functioning as a novel tumor suppressor regulating AKT signaling activation and autophagy as a phosphoinositide-binding protein, being frequently inactivated by promoter methylation in multiple tumors as a potential biomarker. See, e.g., Li et al., Cell Death & Differentiation; 2023 May; 30(5):1166-1183. doi: 10.1038/s41418-023-01129-w. PMID: 36813924. DOI: 10.1038/s41418-023-01129-w.

### Introduction

Chromosome 1p36 deletion has been well defined as a common event for multiple malignancies originating from neurons, epithelium, and hematopoietic cells¹. Through classic genetic studies of this locus, a potent tumor-suppressive region corresponding to 1p36 was defined². Particularly, 1p36.3, which is located at the distal end of 1p, is a tumor suppressor gene (TSG) hotspot or “cancer-gene island”^{1, 2, 3}. Several bonafide TSGs have been identified at this small region, including TP73⁴, CHD5⁵, PRDM16⁶ and AJAP1⁷. These TSGs are involved in tumor initiation and progression and frequently inactivated by either genetic mutations and/or promoter methylation in various tumors.

Long non-coding RNA (lncRNA) has previously been considered to be non-coding as there was a lack of long/obvious open-reading frames (ORFs). Recently with the advancement in deep sequencing, mass spectrometry and bioinformatics techniques^{8, 9, 10}, a subset of lncRNAs with non-canonical small ORFs (sORFs) and high evolutionary conservation has been shown to encode functionally important peptides or small proteins^{11, 12, 13}, although the vast majority of them still remain uncharacterized. Some peptides/proteins encoded by non-canonical ORFs of lncRNAs have been reported to possess important oncogenic or tumor-suppressive functions^{11, 14}, regulating cell proliferation and invasion/metastasis of tumor cells. For examples, the peptides/small proteins encoded by lncRNAs HOXB-AS3¹⁵, LOC90024¹⁶, LINCO00266-1¹⁷, suppress colorectal tumor cell growth and metastasis; the circular form of LINC-PINT encodes an 87-aa peptide that inhibits cell proliferation, stemness and enhancing DNA damage response in glioblastoma¹⁸; an ERα-regulated polypeptide ASRPS encoded by LINC00908 is downregulated in triple-negative breast cancer (TNBC) and associated with its poor survival¹⁹. ASRPS inhibits the angiogenesis of breast tumor cells through the STAT3-VEGF pathway, also as a potential biomarker for TNBC¹⁹. These studies demonstrate the novel functions and importance of lncRNA-encoded peptides/proteins in human disease pathogenesis.

Autophagy is a highly conserved, regular cellular degradation pathway that targets multiple proteins and removes damaged organelles by lysosomal degradation, and thus plays a crucial role in the maintenance of normal cellular homeostasis and survival^{20, 21}. Autophagy is also involved in disease pathogenesis and progression of multiple malignancies, due to the genetic/epigenetic disruption of autophagy regulators^{22, 23, 24}. Loss of Beclin 1/BECN1, a critical regulator of autophagy, through LOH and promoter CpG methylation, has been detected in multiple cancers²⁵. On the other hand, SQSTM1/p62, another autophagy adaptor and marker, is overexpressed in multiple cancers and significantly associated with aggressive features (distant metastasis, poor disease-free survival) and advanced stages^{26, 27}. In certain contexts, induction of autophagy has been shown as a mechanism of tumor suppression, in addition to apoptosis, senescence and cell cycle regulation²⁸. Therefore, autophagy dysregulation frequently contributes to cancer pathogenesis and progression.

In this study, through integrative cancer epigenomics, we identified a 1p36.3 lncRNA KIAA0495 (previously also named as PDAM or TP73-AS1), as a methylated target in multiple tumors. KIAA0495 was frequently silenced by promoter CpG methylation in multiple tumors which could serve as a potential biomarker. We further discovered that the ORF2 of KIAA0495 encoded a small protein (thus named as SP0495), that could be detected both in vivo and in vitro. SP0495 is involved in regulating tumor cell proliferation, apoptosis, autophagy and senescence. We also found that several key oncogenic signaling pathways, including PI3K/AKT/mTOR, NF-κB, and Wnt/β-catenin signaling, were regulated by SP0495. Moreover, we demonstrate that SP0495, as a phosphoinositide-binding protein, represses AKT phosphorylation and downstream signaling activation, and further promotes autophagy via enhancing BECN1 stability to suppress tumorigenesis.

### Results

### Identification of KIAA0495 as a 1p36.3 Transcript Silenced in Tumors Through Integrative Epigenomics

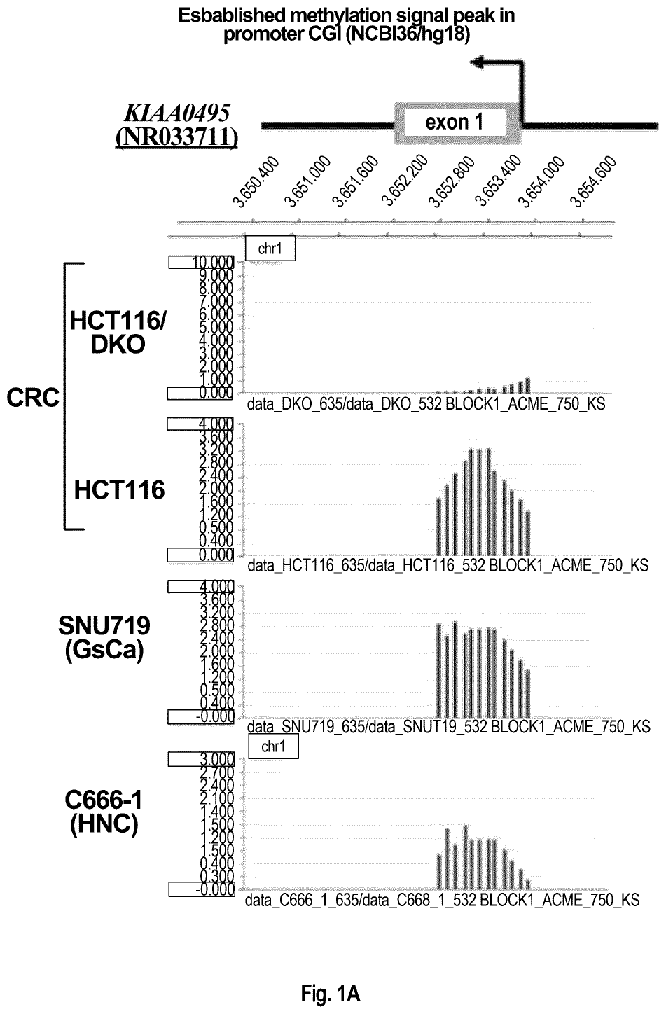

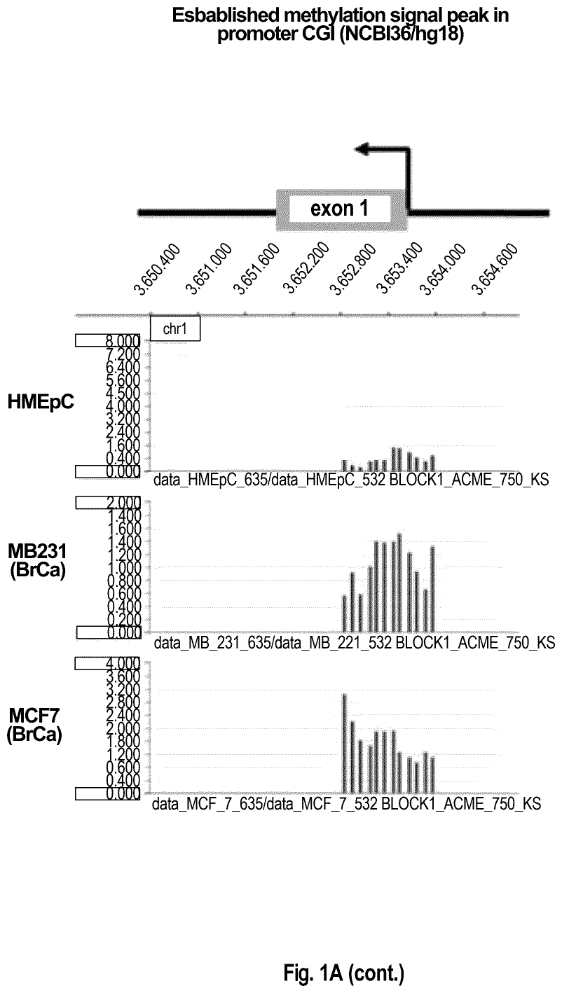

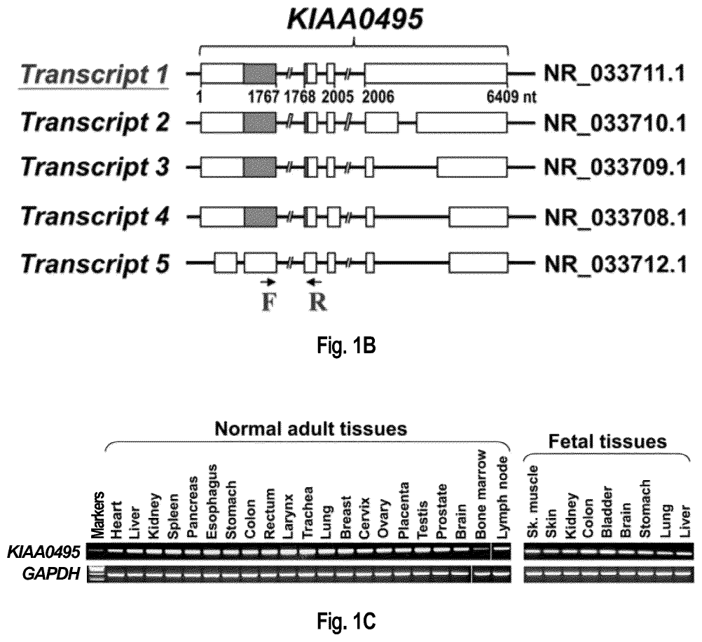

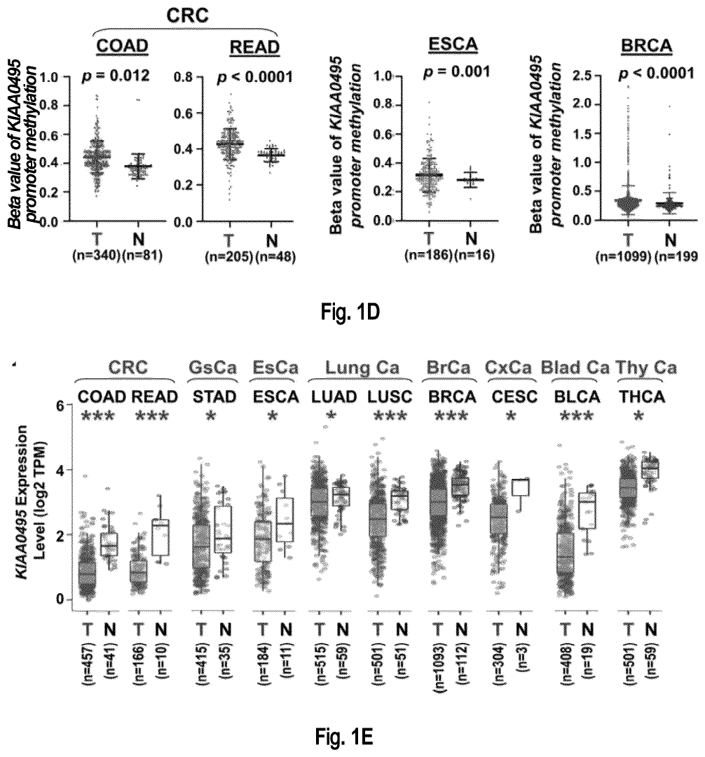

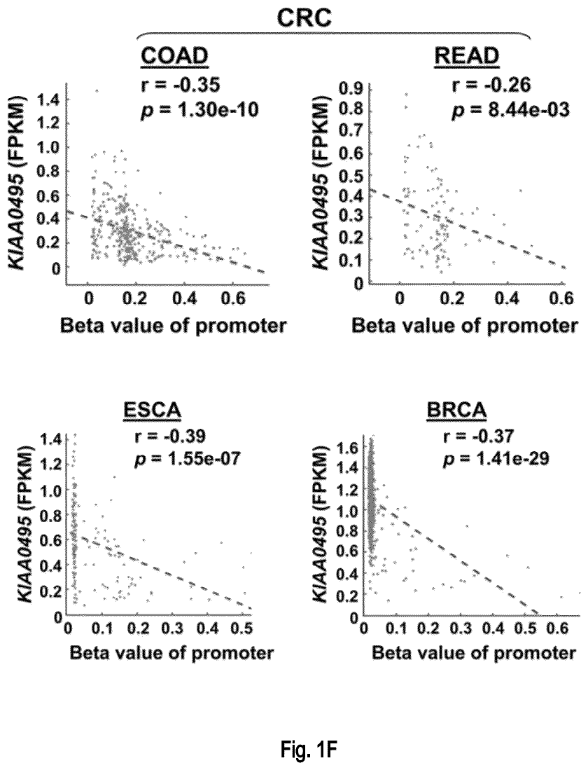

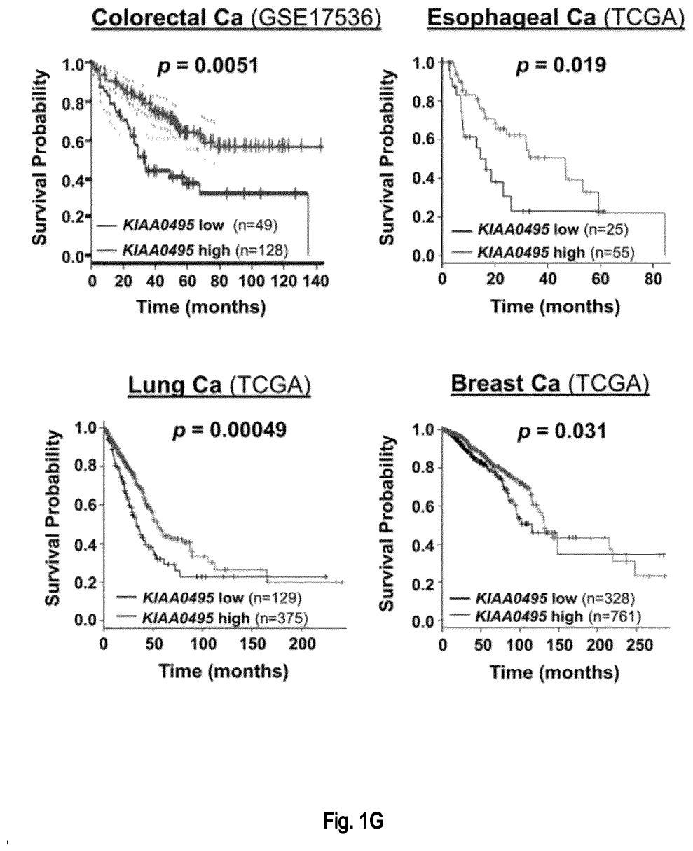

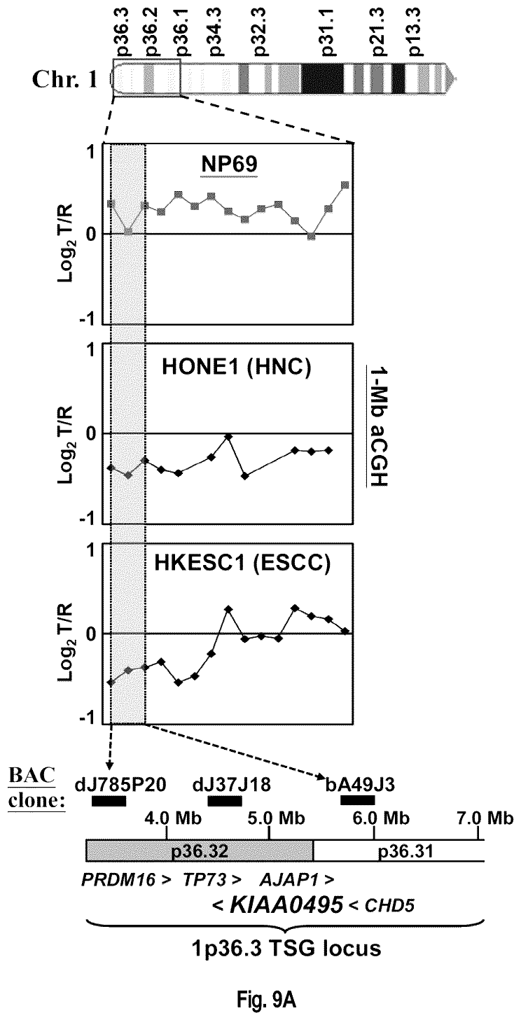

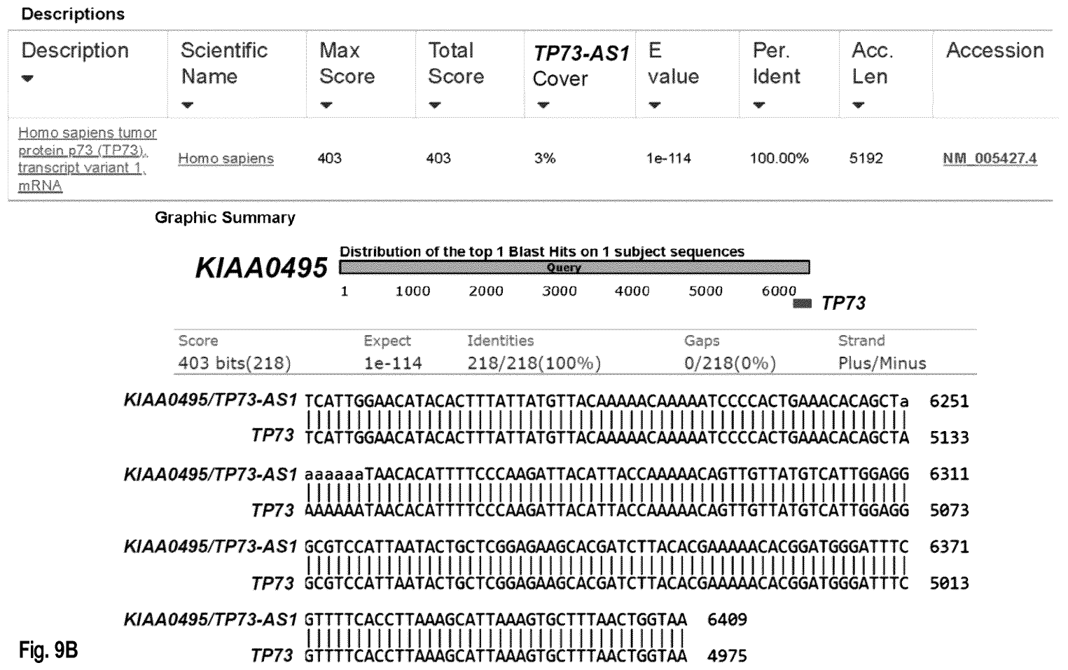

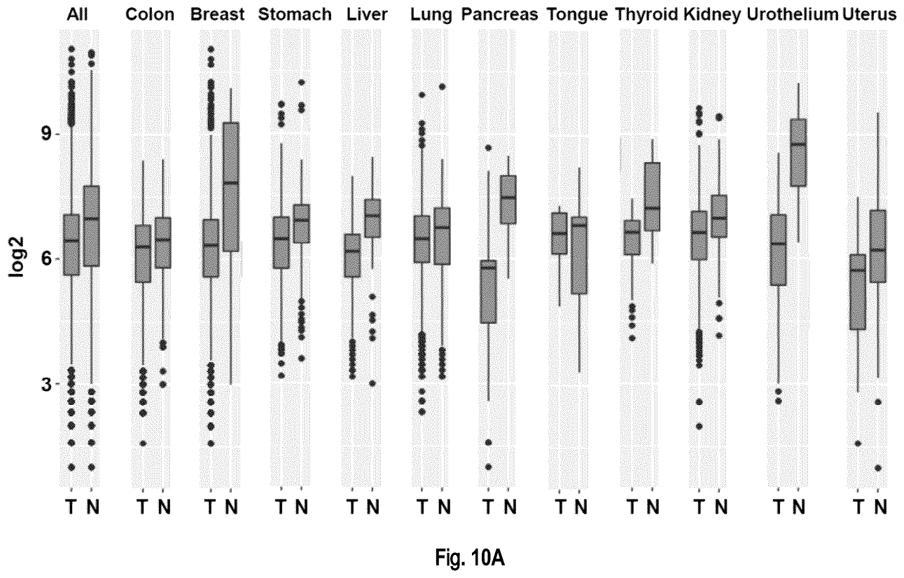

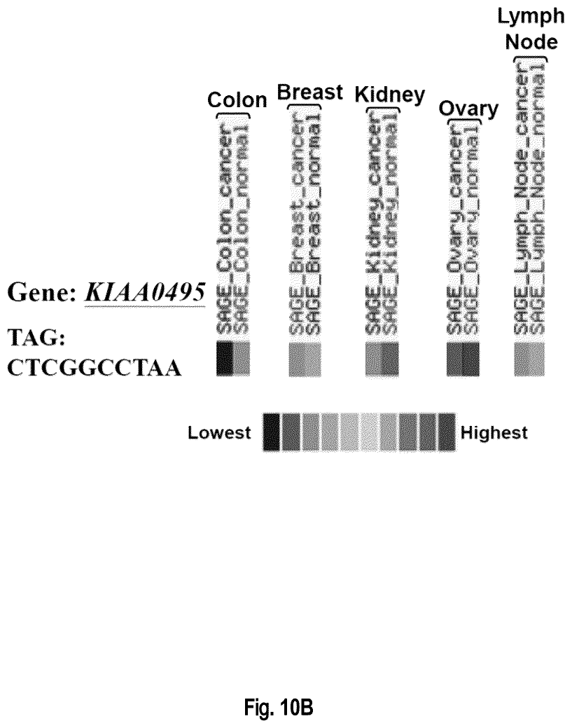

We performed CpG methylome analysis of multiple tumor cell lines of colorectal, gastric, head and neck and breast, and immortalized normal epithelial cells by methylated DNA immunoprecipitation (MeDIP). We detected enriched methylated signals in the promoter region of a previously claimed lncRNA KIAA0495 (NR_033711) in tumor cells of colorectal (HCT116), gastric (SNU719), head and neck (C666-1), and breast (MB231 and MCF7), but not in immortalized mammary epithelial cells (HMEpC) and HCT116 cells with genetic double knockout of DNA methyltransferase DNMT1 and DNMT3B (DKO) (FIG. 1A). Meanwhile independently, using 1-Mb array comparative genomic hybridization (aCGH), we refined a 1p36.3 hemizygous deletion containing KIAA0495 in head and neck (HONE1), and esophageal (HKESC1) carcinoma cell lines (FIG. 9A). Gene copy number analysis using the Oncomine database also revealed that KIAA0495 copy number was reduced in multiple tumors compared to normal tissues (Table 2).

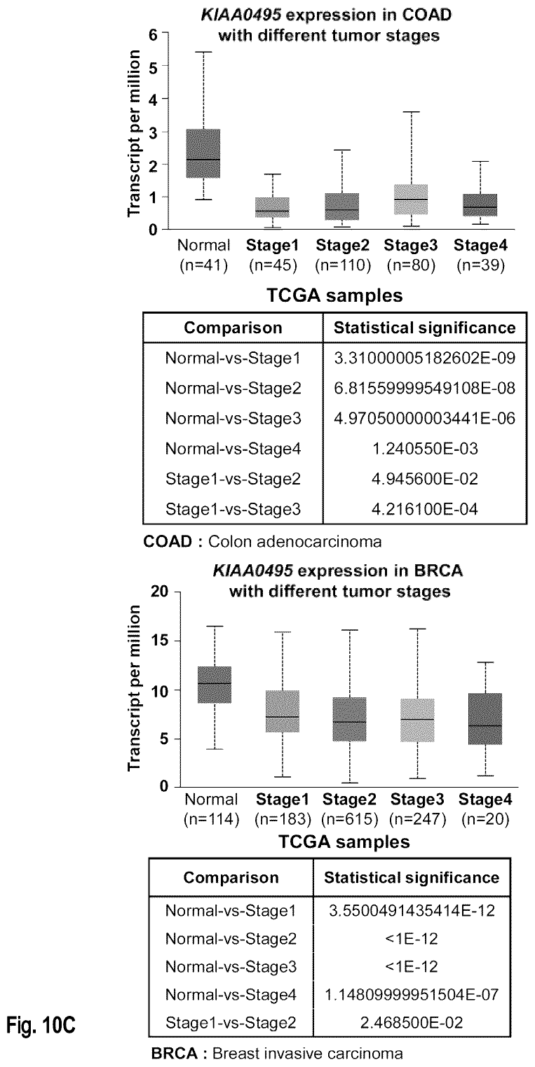

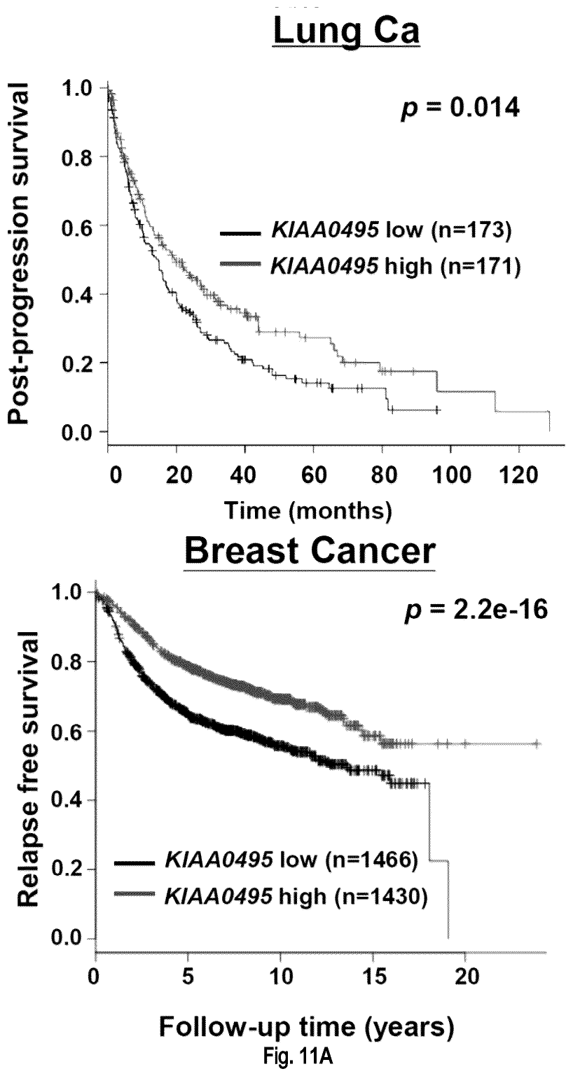

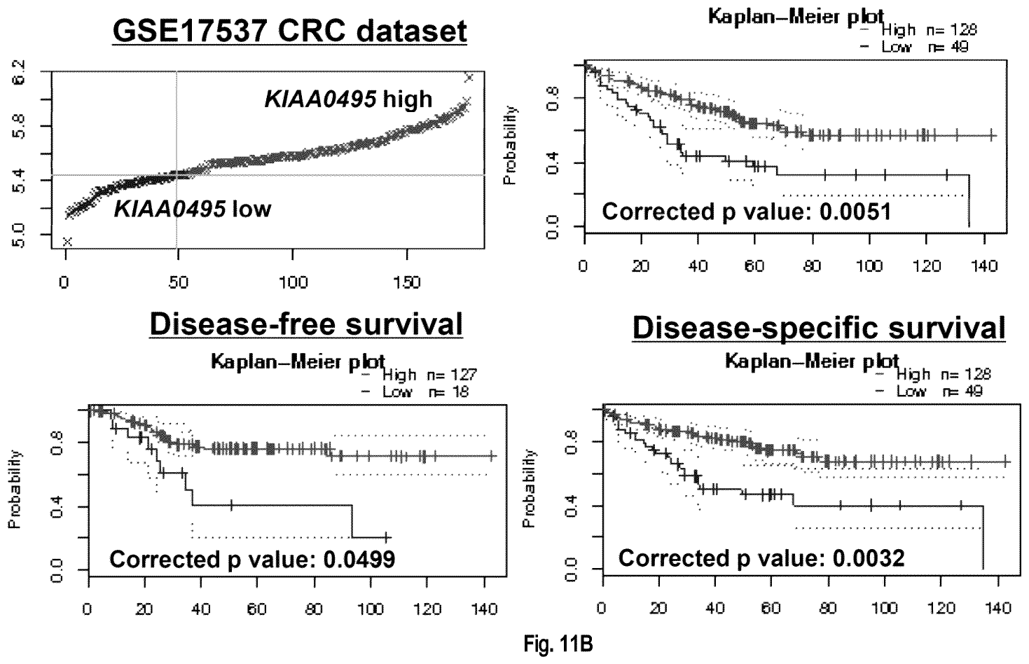

KIAA0495 contains 5 transcripts, with transcript 1 (NR_033711.1) as the longest. First submitted to NCBI database by Kazusa DNA Research Institute (FIG. 1), transcript 1 shares the same exon 1 and 2 with transcripts 2, 3, 4, thus from the same promoter. Although as a novel transcript in the immediate vicinity of TP73, KIAA0495 has only 218-bp complementary sequence homologous to the TP73 transcript in its C terminal, which was previously renamed as TP73-AS1 (FIG. 9B). We further examined KIAA0495 expression in normal human tissues using primers targeting all transcript isoforms by semi-quantitative RT-PCR, and detected its ubiquitous but variable expression in human normal adult and fetal tissues (FIG. 1C). We then retrieved KIAA0495 methylation data of colorectal, esophageal, and breast samples from the Cancer Genome Atlas (TCGA) database, and found significantly higher methylation levels of KIAA0495 in these tumor tissues compared to normal tissues (FIG. 1D). KIAA0495 expression was significantly downregulated in TCGA cohorts of multiple cancers including colorectal, gastric, esophageal, lung and breast (FIG. 1E). Further analyses of Gene Expression (SAGE), GENT2, and Oncomine databases also showed KIAA0495 downregulation in a panel of tumor tissues including digestive, head and neck, and breast cancers (Table 3, FIG. 10). Moreover, KIAA0495 expression was negatively correlated with its promoter methylation in colorectal, esophageal and breast cancer tissues in TCGA Methylation450K datasets (FIG. 1F).

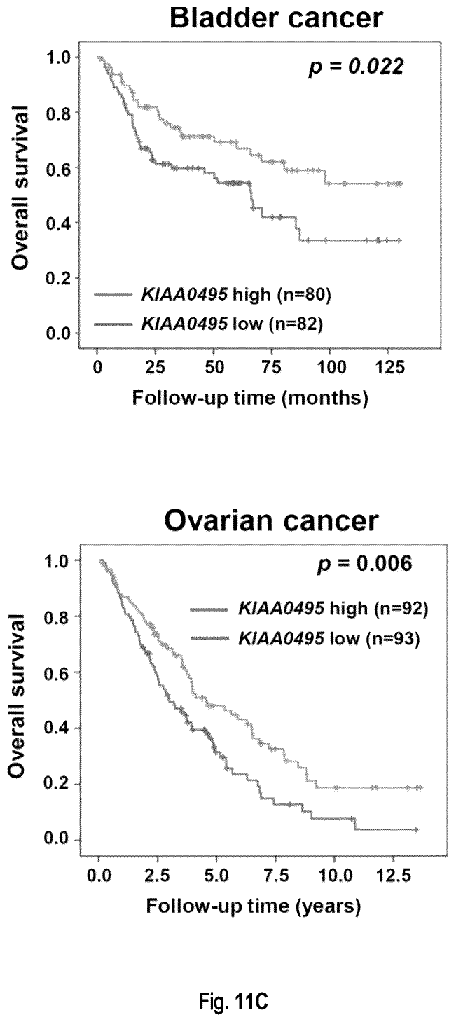

We further found that decreased expression of KIAA0495 was associated with higher-grade tumors in colorectal, breast, and bladder cancer patients (Table 4, FIG. 0C), and poor survival of patients with colorectal, esophageal, lung, breast, bladder, and ovarian cancers (FIG. 1G, FIG. 11). These results indicate that silencing of the 1p36.3 transcript KIAA0495, through epigenetic abnormalities, is common during tumorigenesis and associated with poor survival of cancer patients of multiple tissue origins.

### KIAA0495 Encodes an Endogenously Expressed Small Protein SP0495

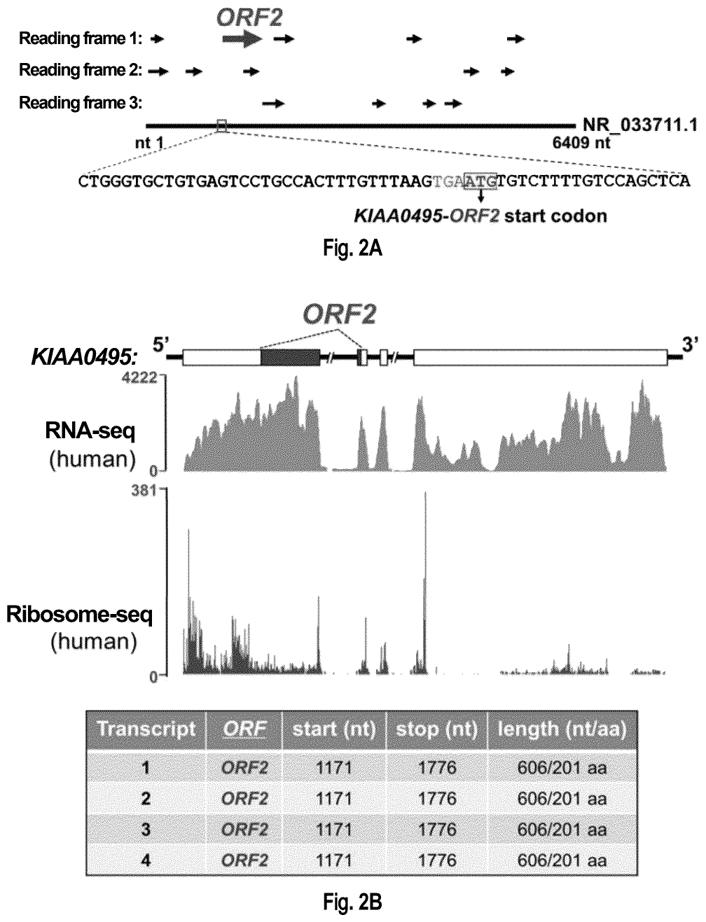

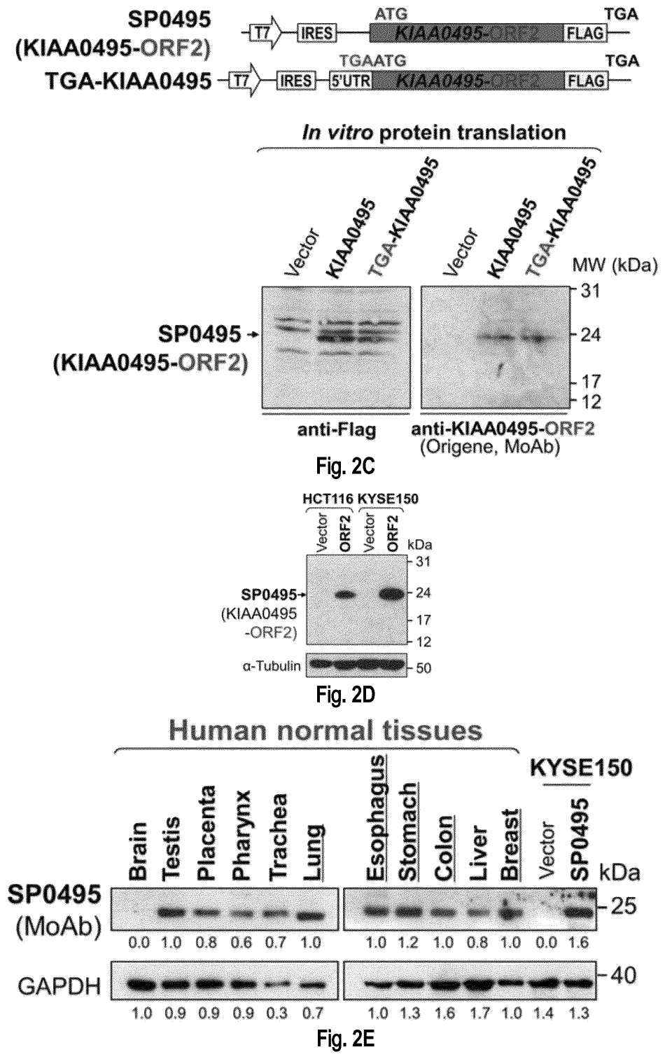

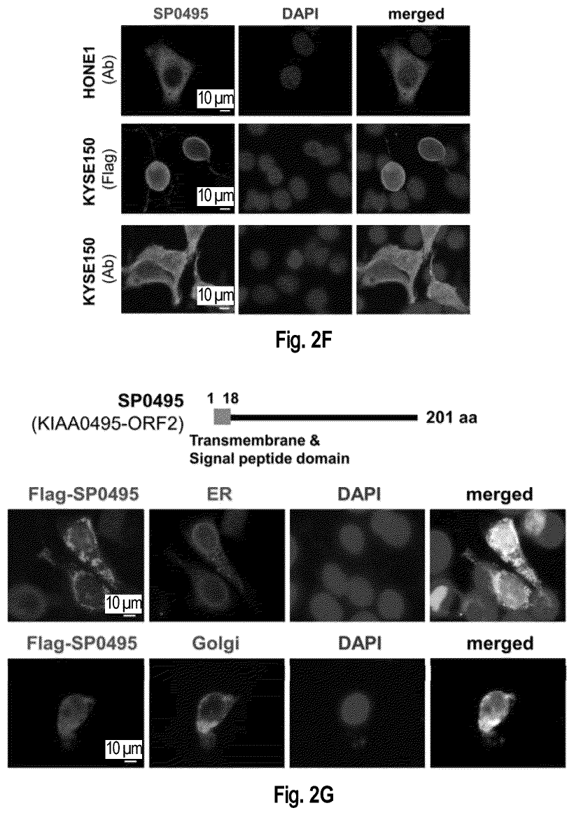

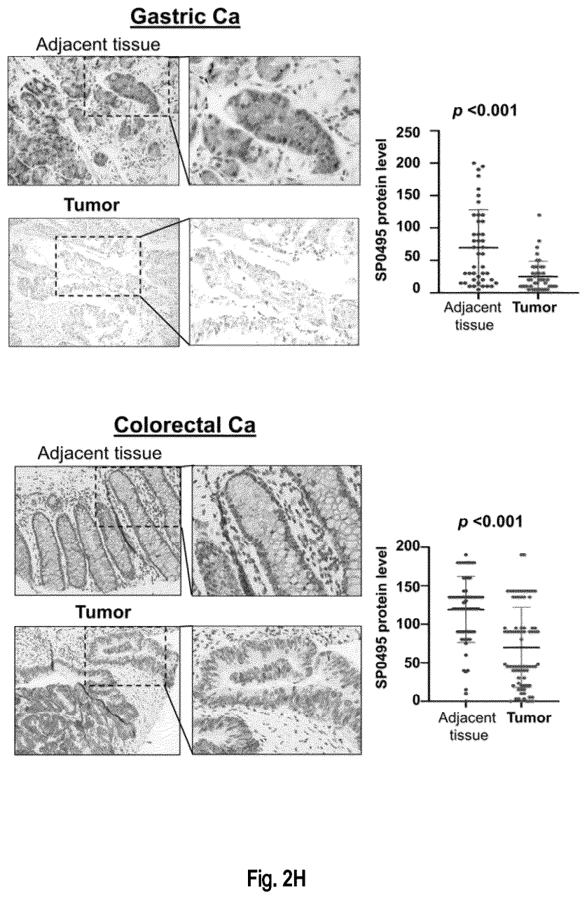

Although KIAA0495 was previously annotated as a lncRNA, using ORFfinder, we discovered a 606-nucleotide non-canonical ORF2 (having the nucleotide sequence set forth in SEQ ID NO:2), which is also the largest ORF with coding potential present in transcripts 1-4, encoding a 201-aa small protein (FIG. 2A, B, having the amino acid sequence set forth in SEQ ID NO:1). Exploring ribosome profiling data using GWIPS-viz shows that KIAA0495-ORF2 is a transcriptionally activated region in human cell lines and tissues studied (FIG. 2B). We thus termed this small protein encoded by KIAA0495-ORF2 as SP0495 (small protein of KIAA0495, amino acid sequence shown in SEQ ID NO:1) hereafter.

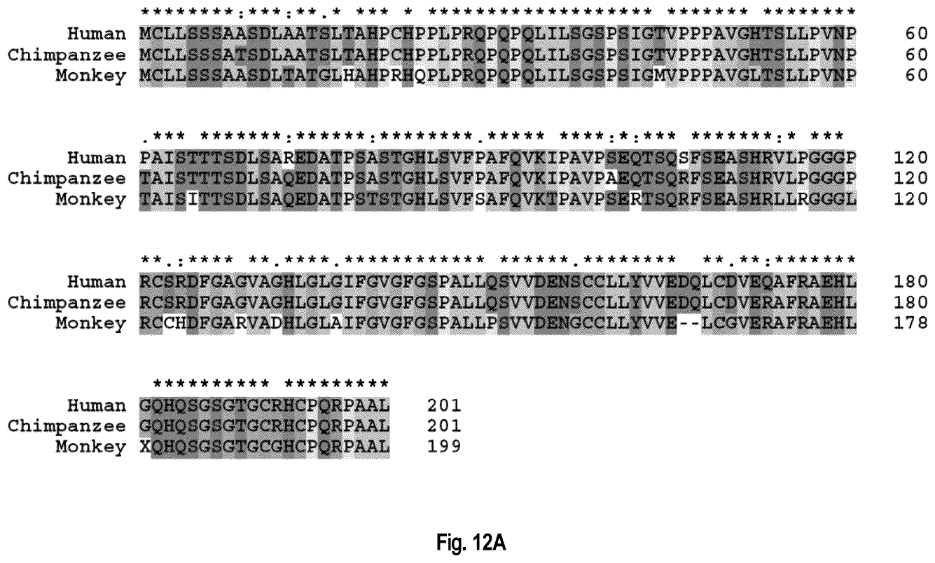

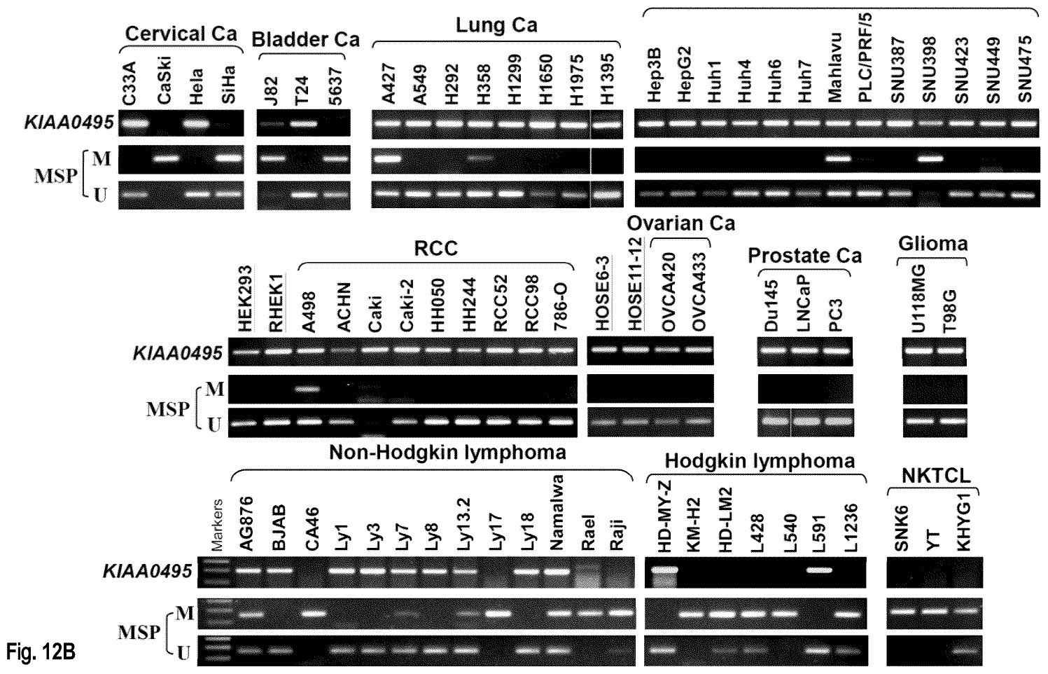

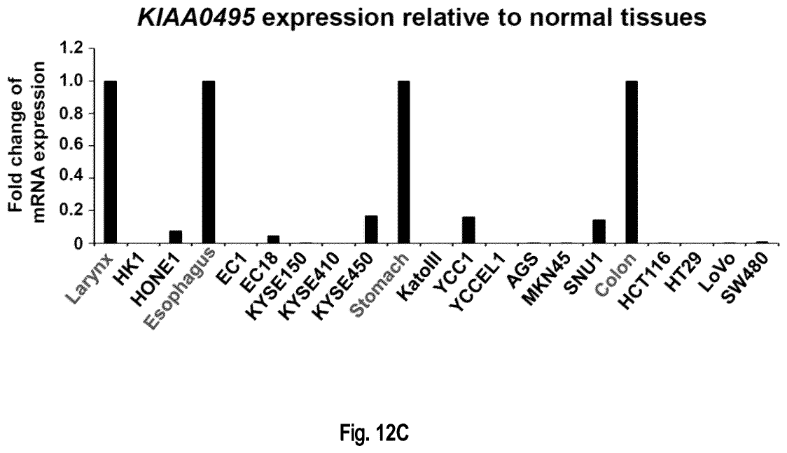

We then generated Flag-fused constructs containing ORF2, with or without a 5′-UTR stop codon (TGA) to confirm the coding potential of the predicted ORF2 (FIG. 2B, C), as a 5′ TGA stop codon is located immediately in front of the ATG codon of ORF2. Results of in vitro protein expression system showed that KIAA0495-ORF2/SP0495 could be translated to protein products at the predicted size˜21 kDa, using both anti-Flag and anti-KIAA0495 antibodies (Origene, clone ID: OTI1F8) (FIG. 2C), indicating that the presence of a 5′-TGA stop codon immediately in front of the ATG codon of ORF2 does not affect its translation efficiency. Furthermore, in ectopically expressing cell line systems, abundant expression of KIAA0495-ORF2/SP0495 protein was consistently detected in two different cell lines (FIG. 2D). We also examined the endogenous expression of SP0495 in normal human tissues, and detected the natural, endogenous presence of SP0495 at the same size with ectopically expressed SP0495 in a panel of human normal tissues using an anti-KIAA0495 antibody, except for brain tissue (FIG. 2E). Bioinformatics alignment showed that human KIAA0495-ORF2/SP0495 protein shares high similarity with that of chimpanzee and monkey, with no homolog identified in other species (FIG. 12A), indicating that it is likely a primate-specific protein.

We further evaluated the subcellular localization of SP0495. Indirect immunostaining revealed that SP0495 is located predominantly in cell cytoplasm in SP0495-endogenously or -exogenously expressed cells (FIG. 2F). Bioinformatics analysis showed that SP0495 contains a transmembrane and signal peptide domain at its N-terminal (1-18-aa) (FIG. 2G). We thus used ER- or Golgi-specific fluorescent plasmid to evaluate colocalization of SP0495 with ER or Golgi in cells, and found that SP0495 was partially localized with ER but completely colocalized with Golgi (FIG. 2G).

Endogenous SP0495 protein expression was further examined in gastric and colorectal tumor tissue samples and adjacent normal tissue samples by immunohistochemistry (IHC) (FIG. 2H). SP0495 expression levels were significantly higher in adjacent non-tumor tissues than gastric and colorectal tumor tissues (FIG. 2H), and upregulation of SP0495 protein levels was correlated with increased tumor size in colorectal tumor tissues (Table 5). Thus, the above results demonstrate that KIAA0495-ORF2 indeed encodes a small protein SP0495, which is endogenously expressed but downregulated in multiple tumor tissues.

### Promoter Methylation of KIAA0495 Leads to its Silencing in Tumor Cell Lines

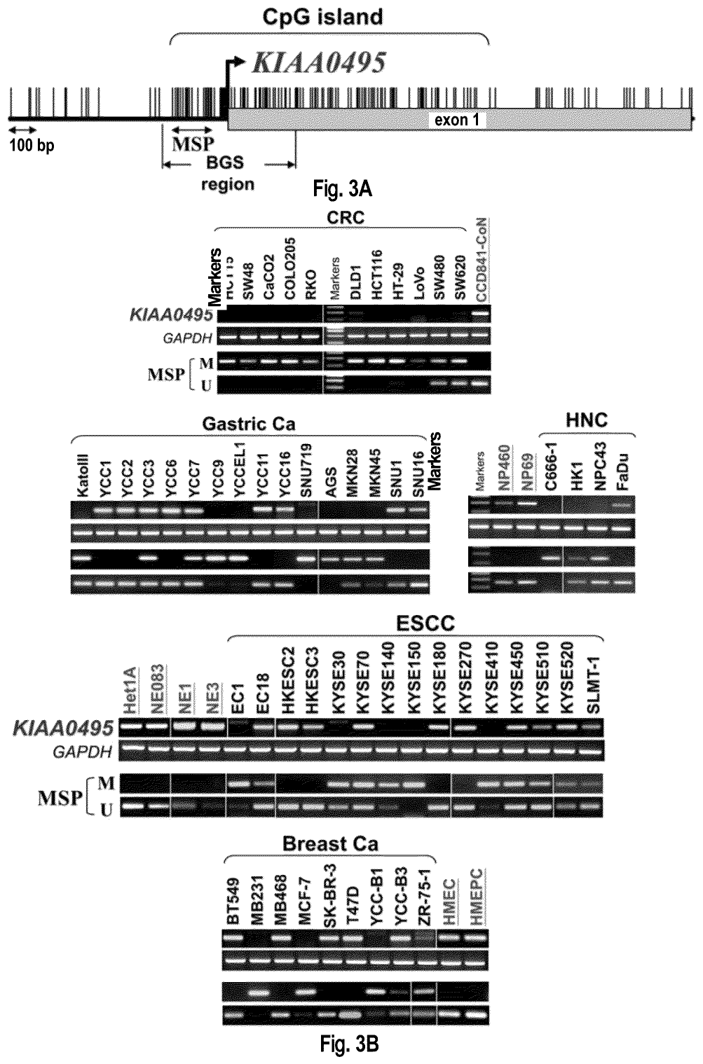

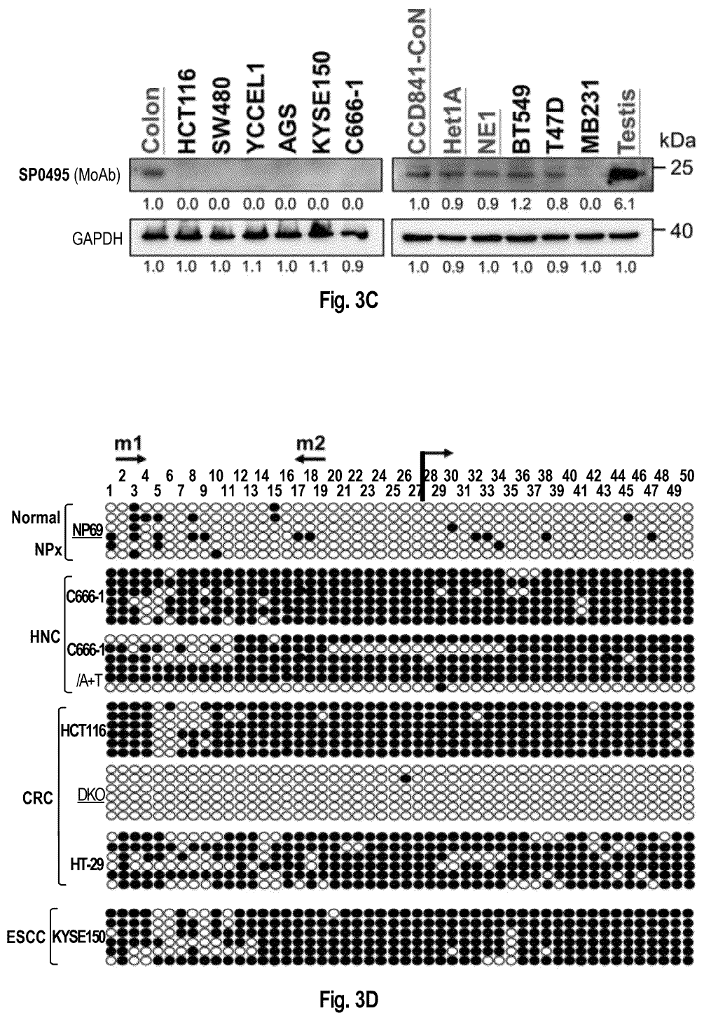

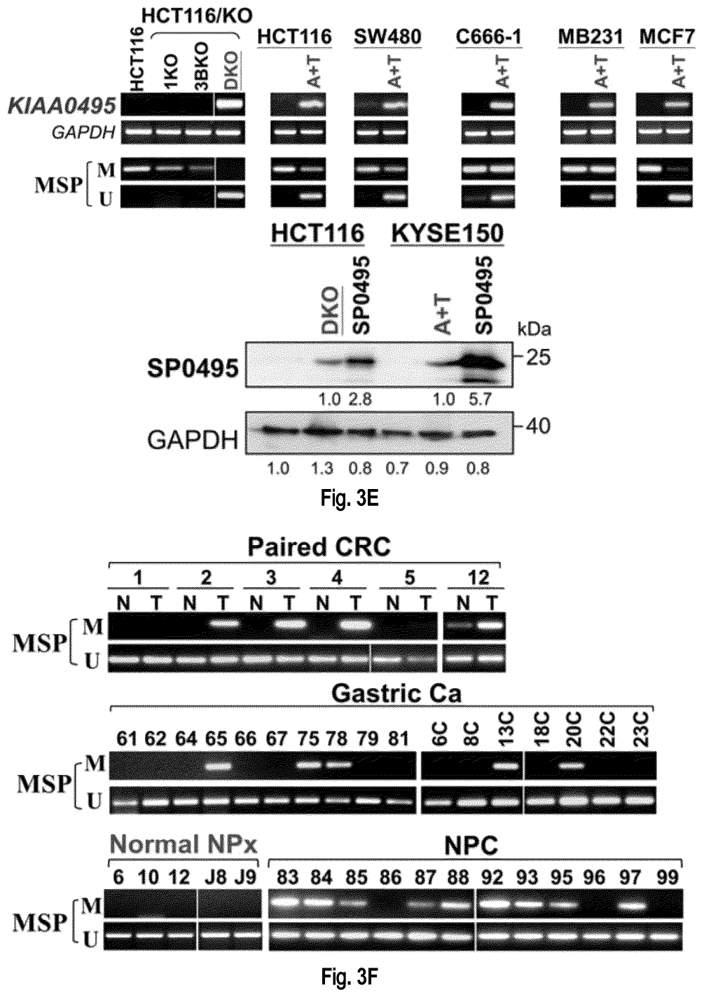

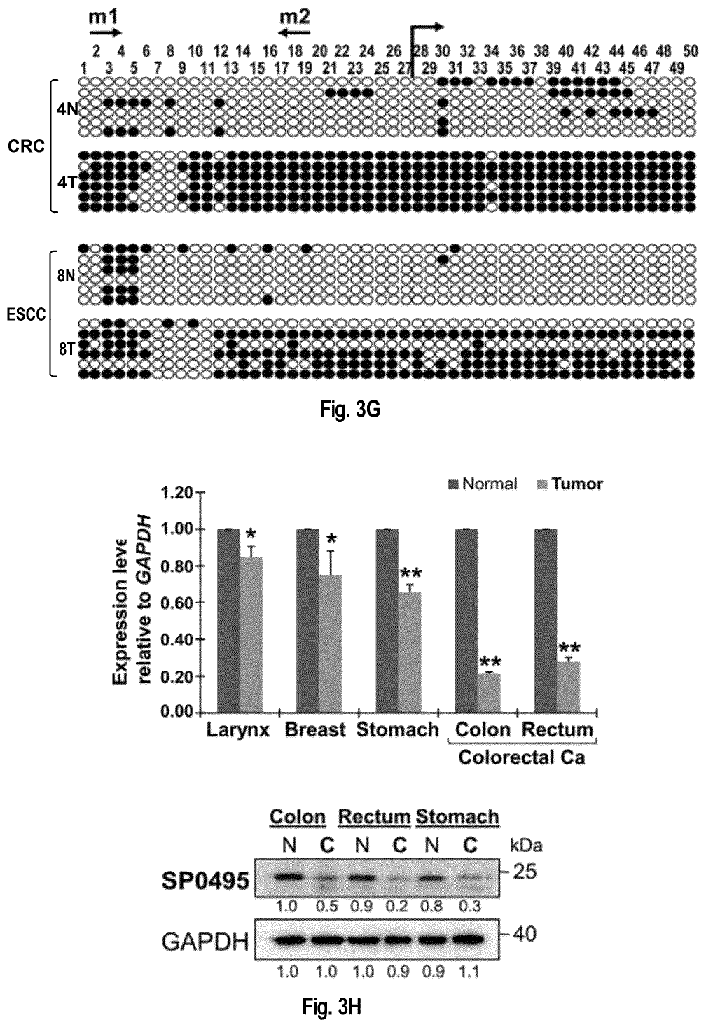

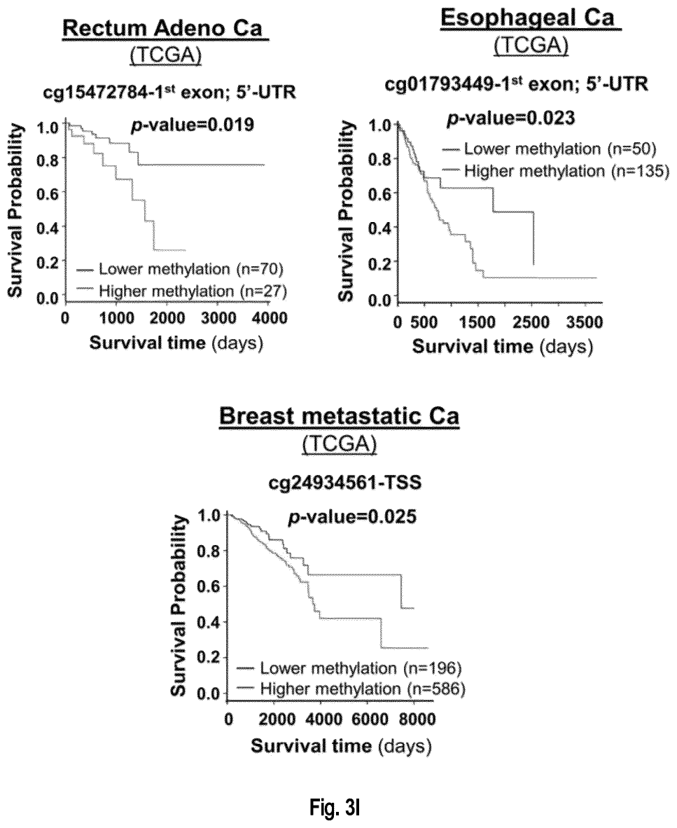

We next investigated the expression levels and regulatory mechanisms of KIAA0495 in tumor cells. KIAA0495 promoter contains a typical CpG island spanning its transcription start site to exon 1 (FIG. 3A), suggesting that it should be regulated by promoter CpG methylation. We examined KIAA0495 expression and promoter methylation in multiple tumor cell lines. Semi-quantitative RT-PCR showed that, in contrast to its abundant expression in normal tissues (FIG. 1C) and normal immortalized cell lines (FIG. 3B), KIAA0495 was frequently silenced or downregulated in tumor cell lines of colorectal (11/11), gastric (7/16), head and neck (3/4), esophageal (11/17), breast (4/9), bladder (2/3), cervical (2/4) cancers, as well as non-Hodgkin and Hodgkin lymphomas (12/23) (FIG. 3B, FIG. 12B). Similar results were confirmed by real-time RT-PCR at the RNA level and Western blot at the protein level (FIG. 12C, FIG. 3C). KIAA0495 silencing/downregulation was uncommon in other cell lines of lung, liver, renal, ovarian, prostate and brain tumors (FIG. 12B).

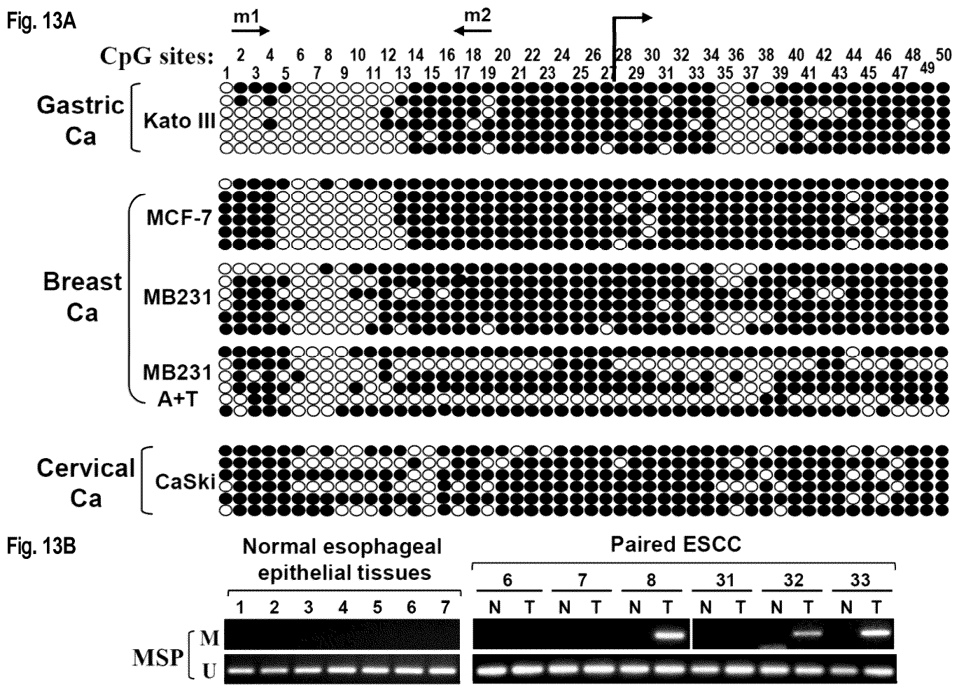

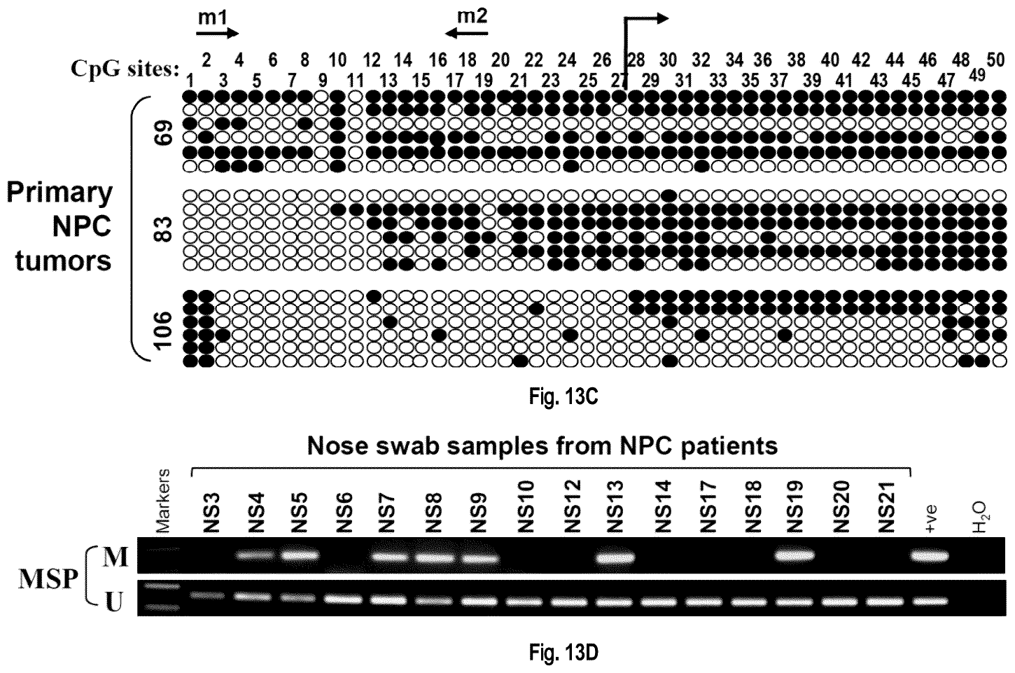

We next examined the methylation status of KIAA0495 by methylation-specific PCR (MSP). We detected methylated promoters in cell lines with decreased or silenced KIAA0495 expression, and no methylation was found in normal or immortalized cell lines (FIG. 3B, Table 1). KIAA0495 methylation was less frequently observed in lung, liver, renal, ovarian, prostate and brain cancers, consistent with its expression status in these tumors (FIG. 12B). We validated the MSP results in several cell lines using high-resolution bisulfite genomic sequencing (BGS) analysis of 50 individual CpG sites within the KIAA0495 CGI. BGS results were consistent with those of MSP, in which densely methylated CpG sites were only detected in methylated cell lines, but not unmethylated normal cell line NP69 (FIG. 3D, FIG. 13A). Thus, the KIAA0495 promoter methylation status is well correlated with its expression levels in normal and tumor cell lines.

We further investigated whether promoter methylation directly contributes to KIAA0495 silencing, using DNMT1 and DNMT3B double knock-out HCT116 cells (HCT116/DKO). Compared to wild-type HCT116 cells with completely silenced KIAA0495, expression of KIAA0495 was significantly reactivated in HCT116/DKO cells, along with concomitant full demethylation of the promoter (FIG. 3E). After demethylation treatment with DNA methyltransferase (DNMT) inhibitor Aza, alone or together with histone deacetylase (HDAC) inhibitor Trichostatin A (TSA), KIAA0495 expression was dramatically restored in several silenced tumor cell lines, along with the significant increase in unmethylated promoter alleles (FIG. 3E). Further high-resolution BGS analysis confirmed the demethylation of the KIAA0495 promoter after pharmacologic or genetic demethylation (FIG. 3D, FIG. 13A). These results indicate that KIAA0495 is regulated by promoter CpG methylation in normal and tumor cells, by DNMT1 and DNMT3B together, like other bonafide TSGs which we and others previously characterized^{29, 30}.

To assess whether genetic alteration, such as mutation, also inactivates SP0495 in tumors, we sequenced all KIAA0495-ORF2 coding exons in a panel of tumor cell lines but found no mutation in any of the 18 cell lines examined (Table 6). These results suggest that genetic point mutation of SP0495 is likely very rare and that epigenetic alteration is the predominant mechanism of its disruption in tumors.

### KIAA0495 Promoter Methylation is Common in Multiple Primary Tumors

To evaluate whether KIAA0495 promoter methylation in tumors is of clinical significance for developing as a biomarker for cancer detection and prognosis prediction, we examined KIAA0495 methylation in a series of primary tumors. KIAA0495 methylation was frequently detected in primary tumors of colorectal (14/23, 61%), gastric (15/51, 30%) and nasopharyngeal (28/48, 58%) cancers, but less in esophageal (7/46, 15%) and breast (3/40, 7.5%) cancers (FIG. 3F, FIG. 13C). In contrast, no normal nasopharyngeal (0/6), esophageal (0/7), and gastric (0/4) tissue showed KIAA0495 methylation (FIG. 3F, Table 1). Among adjacent normal tissues, no esophageal normal tissue (0/46) and only two normal colorectal tissues (2/12, 17%) showed methylation. BGS analysis confirmed the dense methylation in tumors, but not in paired normal tissues (FIG. 3G and FIG. 13C). These results demonstrate that KIAA0495 methylation is a frequent and tumor-specific event in multiple solid tumors. In agreement, qRT-PCR results also showed that KIAA0495 expression was reduced dramatically in tumor tissues compared with adjacent normal tissues, and further confirmed by Western blot (FIG. 3H). Furthermore, we detected KIAA0495 methylation in 7/16 (44%) nose swab samples from NPC patients (FIG. 13D), indicating that it could be developed as an epigenetic biomarker for non-invasive cancer diagnosis.

Clinically, through analyzing TCGA cancer dataset, higher KIAA0495 promoter methylation level is significantly associated with poor outcomes of patients with rectum, esophageal, and breast cancers (FIG. 3I). These findings demonstrate the translational value of tumor-specific KIAA0495 promoter methylation as a cancer biomarker, and also the important role of KIAA0495 inactivation during tumor pathogenesis.

### Small Protein SP0495 Functions as a Tumor Suppressor

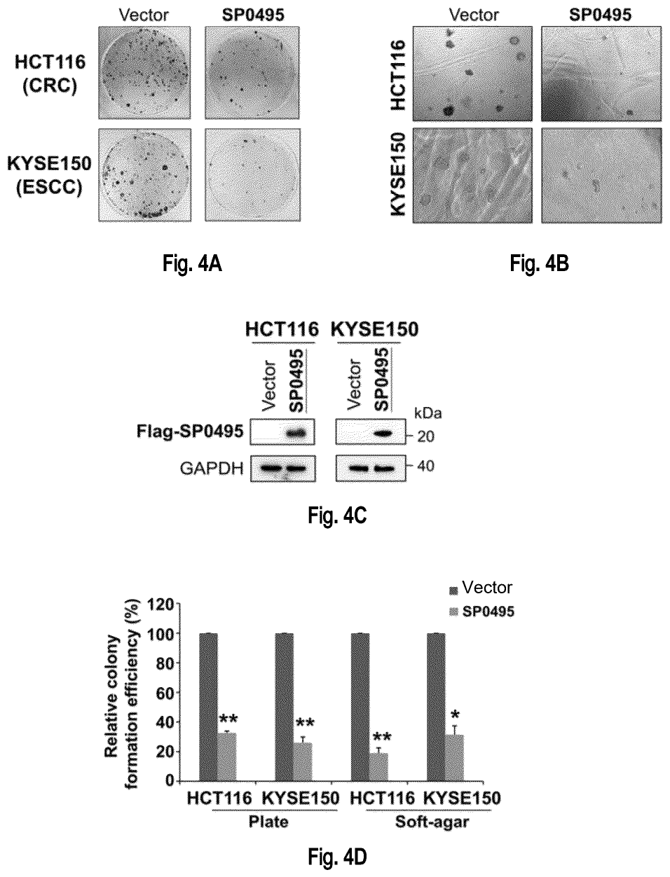

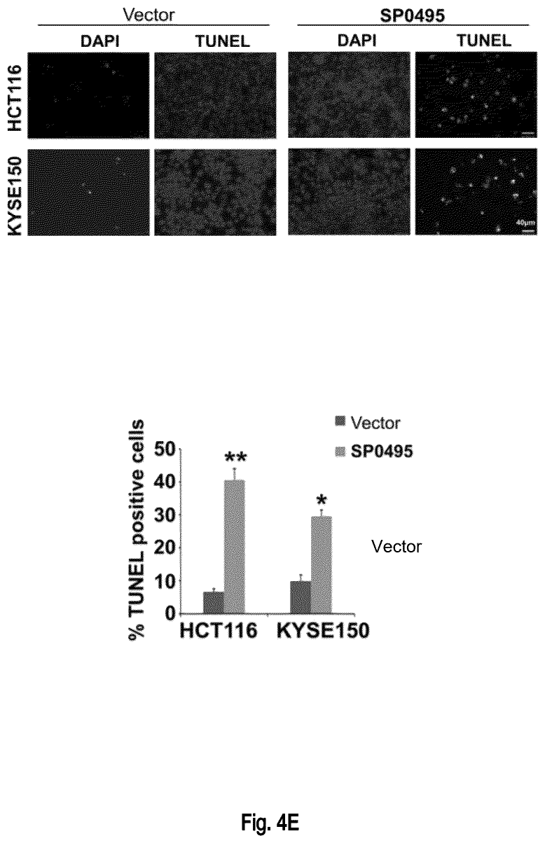

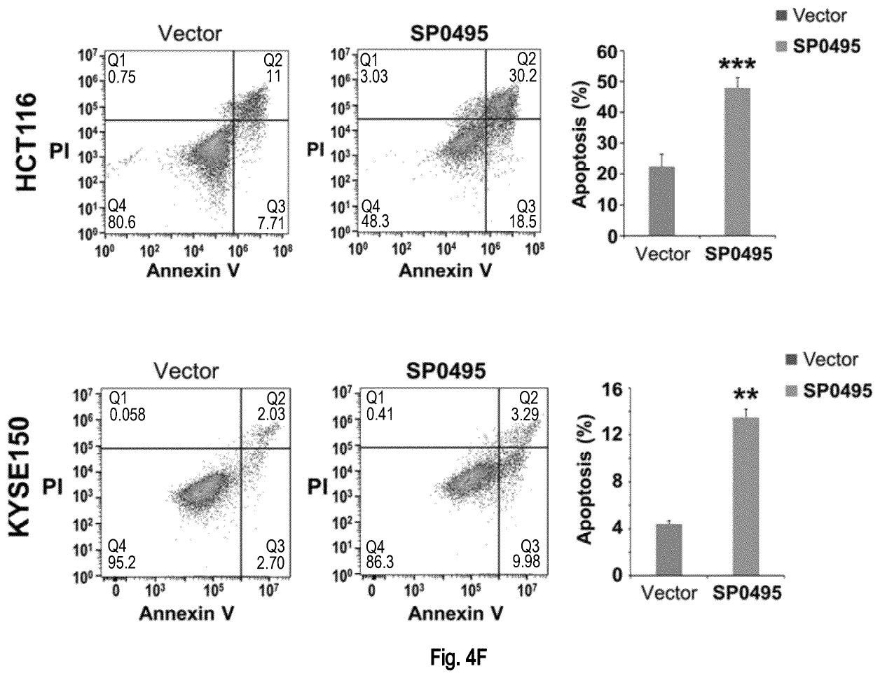

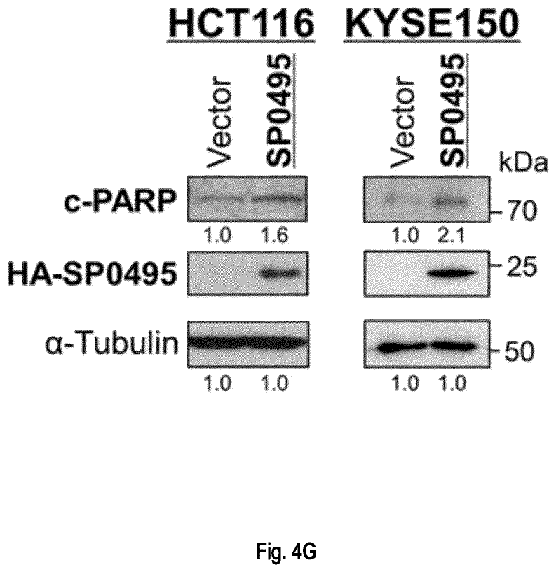

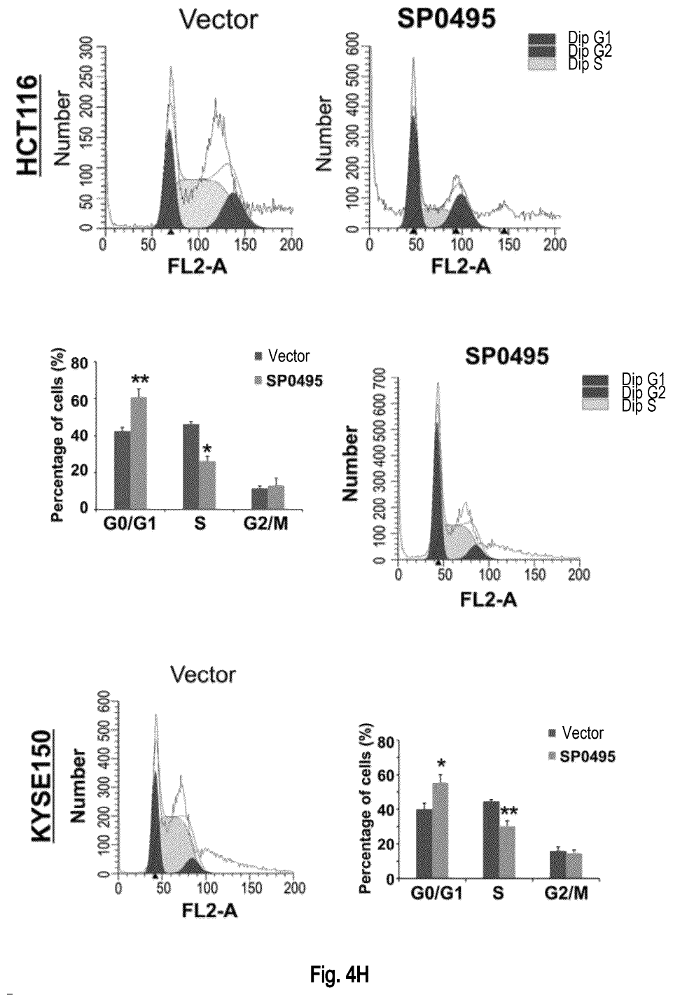

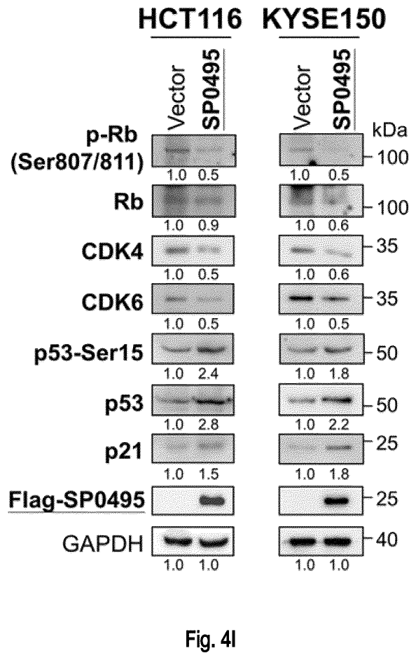

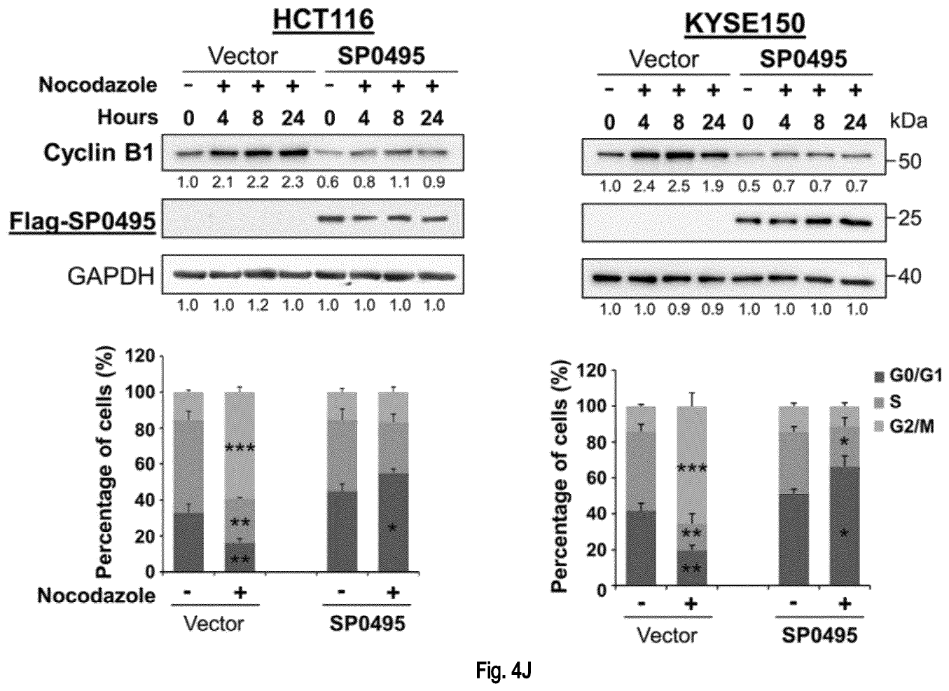

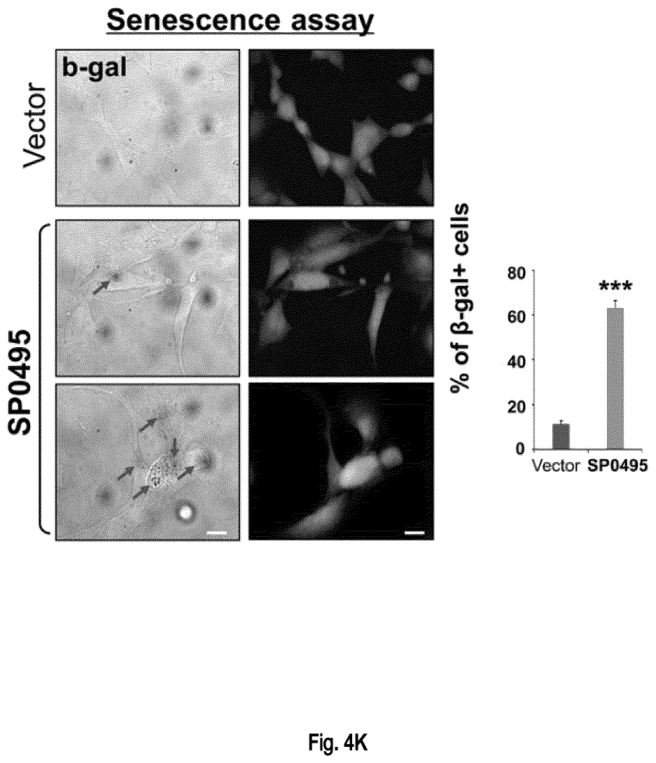

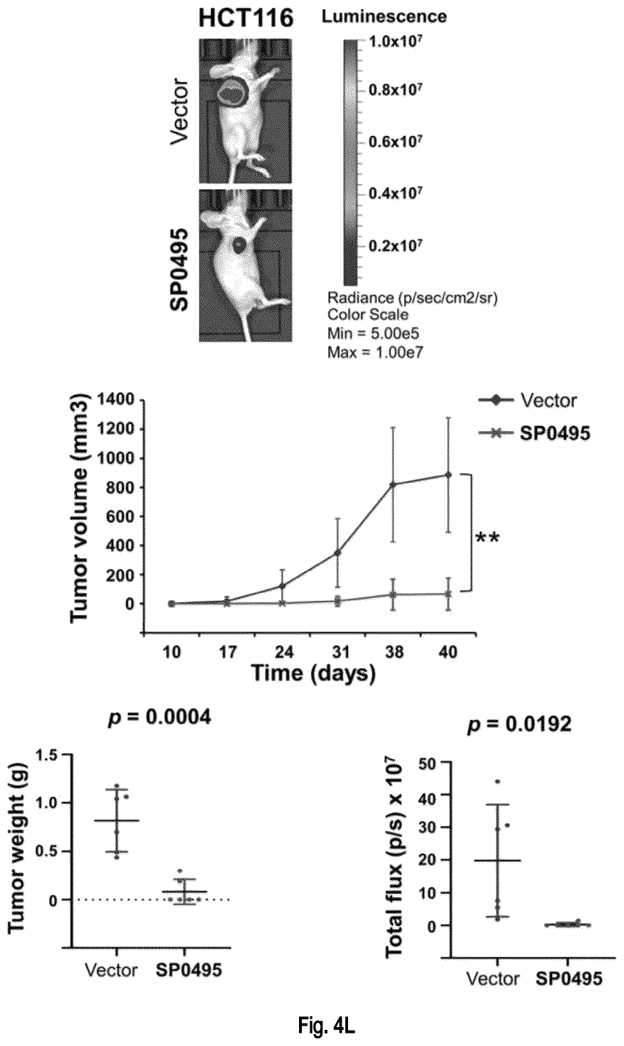

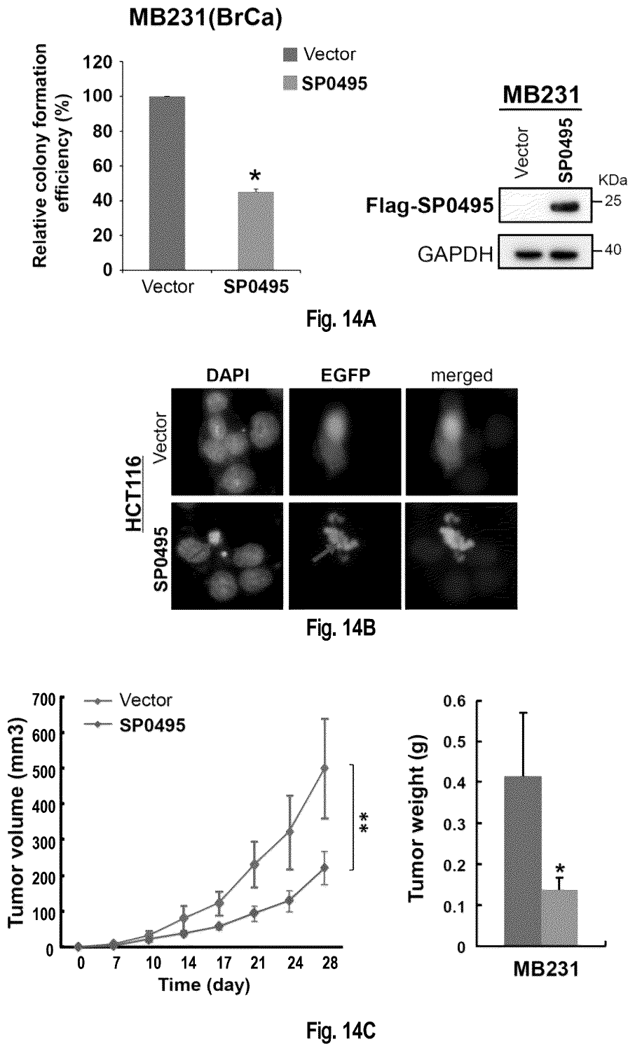

Frequent KIAA0495 methylation in multiple carcinomas implies tumor-suppressive functions of its encoded small protein SP0495. To test this, we firstly examined its growth inhibitory effect on tumor cells by colony formation assays. Compared to controls, significant reduction of colony numbers and sizes were observed in cells stably expressing SP0495, in both monolayer and soft-agar culture colony formation assays (FIG. 4A-D, FIG. 14A), indicating that SP0495 inhibits the anchorage-dependent and -independent growth of tumor cells.

We further explored the underlying mechanisms of tumor suppression mediated by SP0495. We found that ectopic SP0495 expression induced apoptosis of HCT116 and KYSE150 tumor cells, as demonstrated by TUNEL assay (FIG. 4E). Additionally, flow cytometry analysis by Annexin V assay showed that the proportion of apoptotic cells was significantly higher in SP0495-expressing HCT116 and KYSE150 cells than in control cells (FIG. 4F). Consistently, 4,6-diamidino-2-phenylindole (DAPI) staining revealed chromatin condensation and nuclear rupture in SP0495-expressing tumor cells—a hallmark of apoptotic cell death (FIG. 14B). We also observed increased expression of the apoptotic indicator—cleaved PARP, in SP0495-expressing cells as compared to vector controls (FIG. 4G). We then determined whether SP0495 has an impact on cell cycle progression. We found that the tumor cell cycle pattern was altered by SP0495, with significant increase of cell proportion in G1/S phases and decrease of cell population in S phase (FIG. 4H). We further examined the effect of SP0495 on cell cycle-related protein expression, and found that G1-S associated proteins (phosphorylated Rb, CDK4, and CDK6) were downregulated after SP0495 expression, while the expression levels of p53 and p21 proteins as well as phosphorylated p53 (at Ser15) were increased after SP0495 expression (FIG. 4I). To further explore how SP0495 restoration modifies cell cycle, we treated cells with nocodazole to capture cells in G2/M. Western blot analysis of cyclin B1, a key cell cycle regulator of G2/M transition, showed significant increase of protein expression in control cells after nocodazole treatment, but with only minor changes in SP0495-expressing cells (FIG. 4J). Consistently, we observed that control cells after nocodazole treatment mainly accumulated in G2/M, while SP0495 overexpressing cells were still delayed mainly in G1 phase (FIG. 4J). These data indicated the arrest of G1/S phase progress by SP0495 in tumor cells.

Moreover, as p53 and p21 are both linked to cell senescence, we also detected the impact of SP0495 expression on cell senescence. We detected induction of cell senescence in SP0495-expressing immortalized normal cells by staining for senescence-associated β-galactosidase (SA-β-gal). Elevated β-galactosidase staining was observed in SP0495-expressing cells (FIG. 4K), indicating that cell senescence induction is another growth-suppressive mechanism mediated by SP0495. These findings suggested that SP0495 could function as a tumor suppressor through inducing apoptosis, G1/S cell cycle arrest and cell senescence in tumor cells.

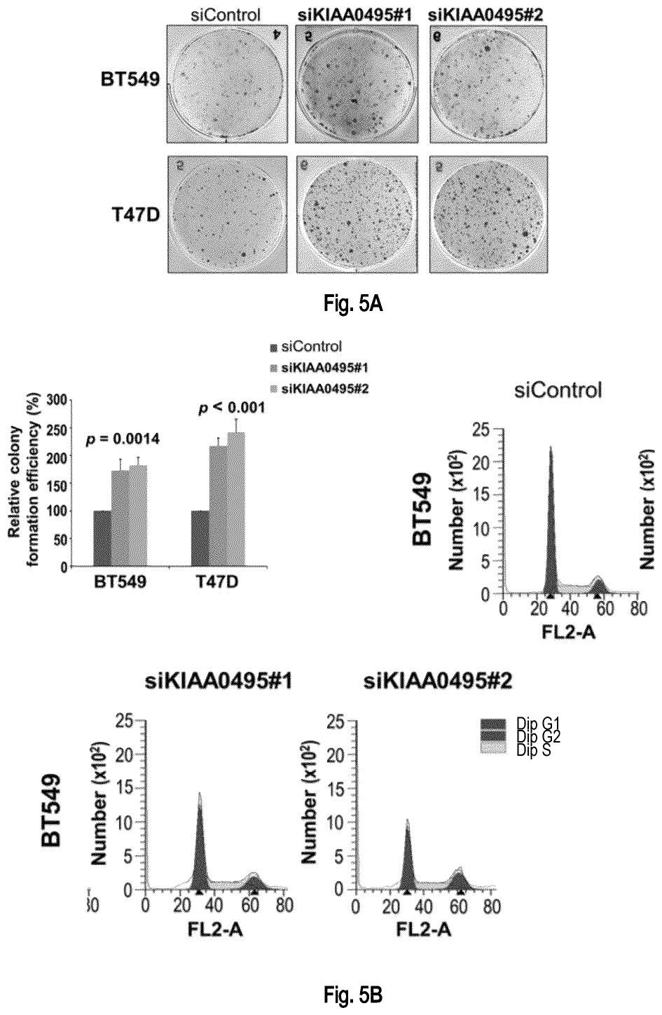

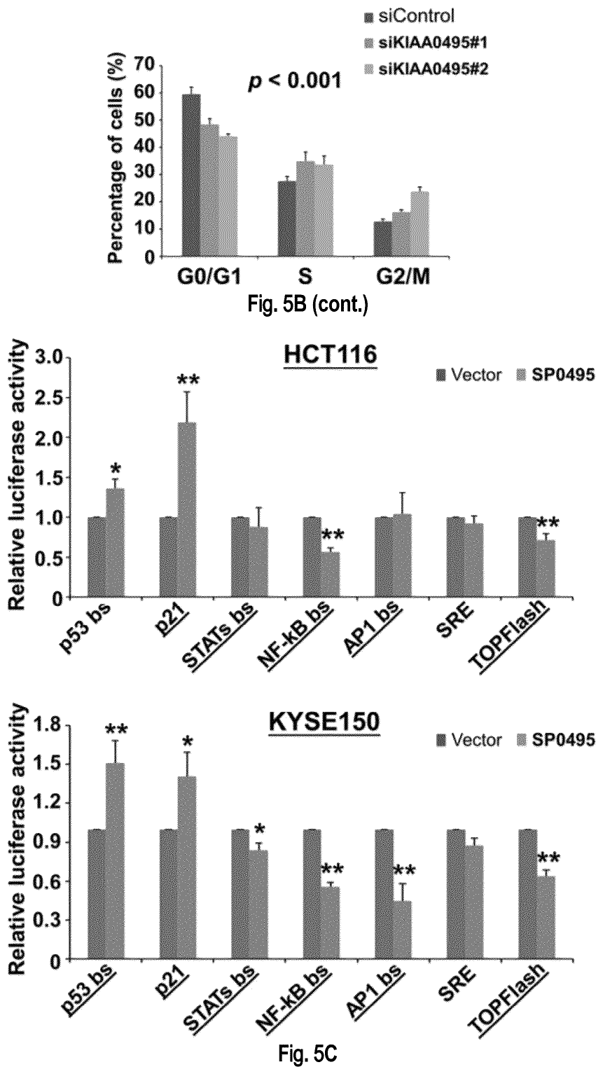

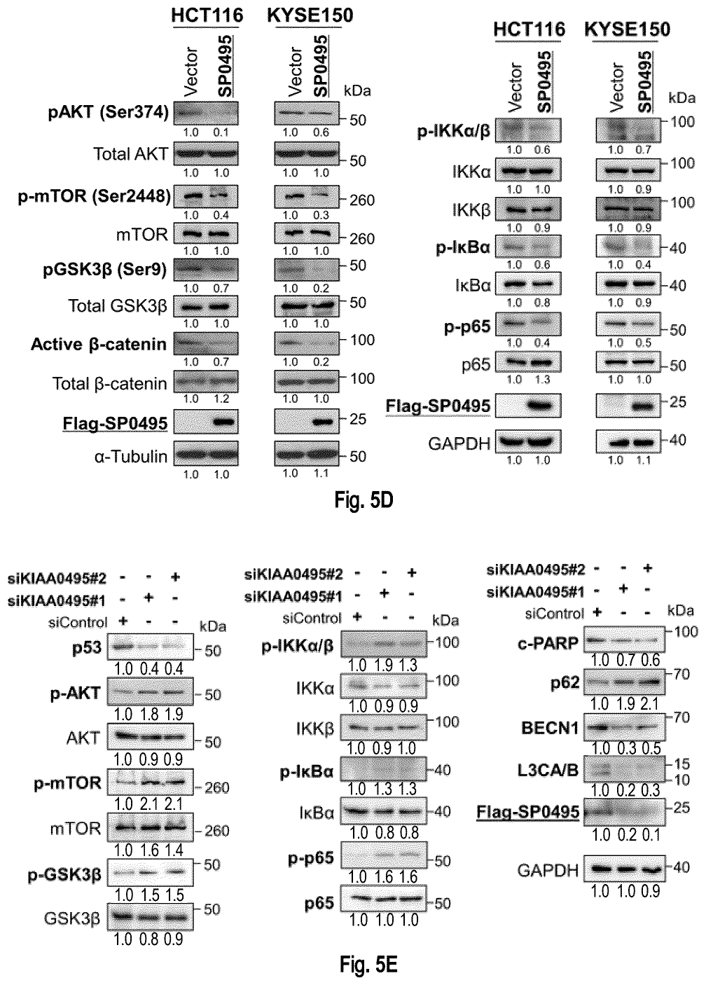

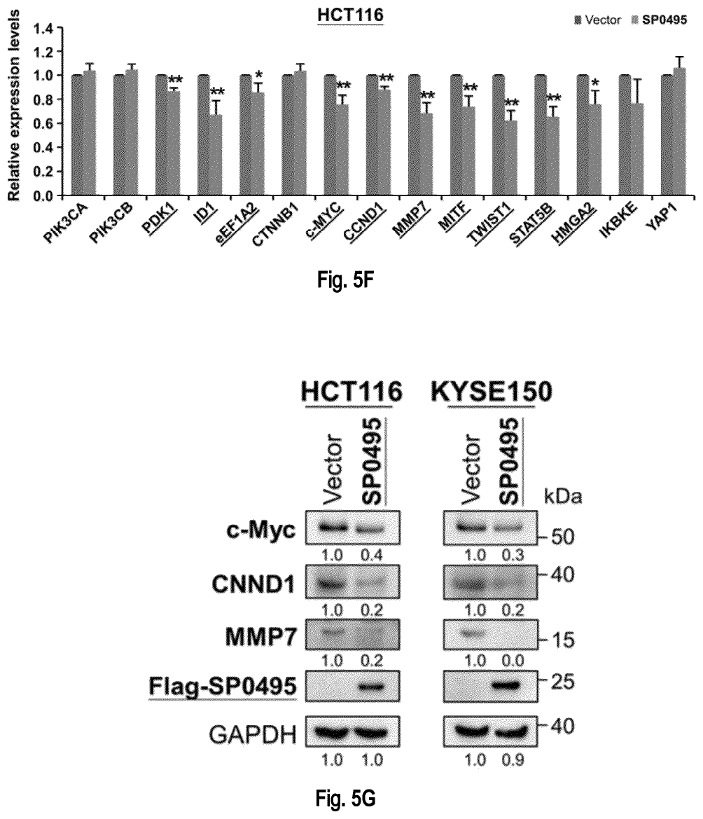

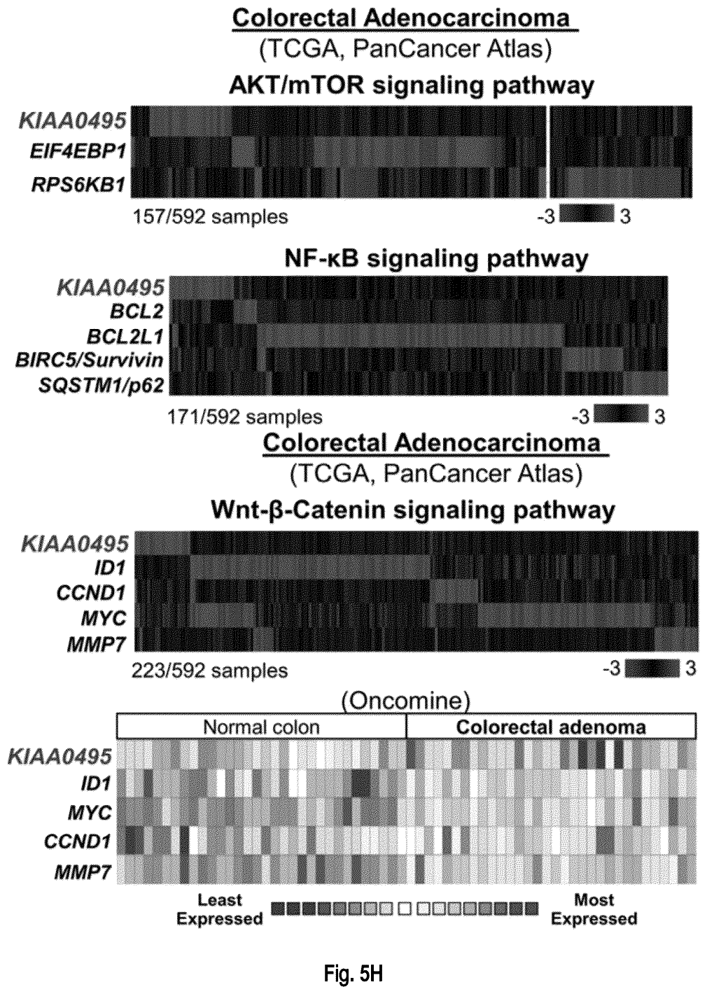

A nude mice animal model was used to investigate whether SP0495 could suppress tumor formation in vivo. HCT116 and MB231 tumor cells with stably expressed SP0495 or control vector were injected into nude mice, with tumor formation efficiency monitored across different time points. SP0495 overexpression significantly decreased tumor growth and average tumor weight of HCT116 and MB231 xenografts in nude mice (FIG. 4L, FIG. 14C). Compared with the control group, the fluorescence intensity in SP0495-expressing HCT116 cell group decreased significantly (FIG. 4L), together with slower tumor growth and reduced average tumor weight (FIG. 4L, FIG. 13D). We further tested the tumor-suppressive functions of SP0495 under siRNA knock-down. We found that increased tumor cell growth through knockdown of SP0495 by siRNAs in BT549 and T47D cells, accompanied by reduced proportion of G0/G1 cells and elevated proportion of S and G2/M cells, indicating that SP0495 knockdown could revert the proliferation suppression and G1/S arrest induced by SP0495 (FIG. 5A, B). Thus, SP0495 indeed functions as a tumor suppressor in tumorigenesis.

### SP0495 Suppresses AKT and Other Oncogenic Signaling Pathways

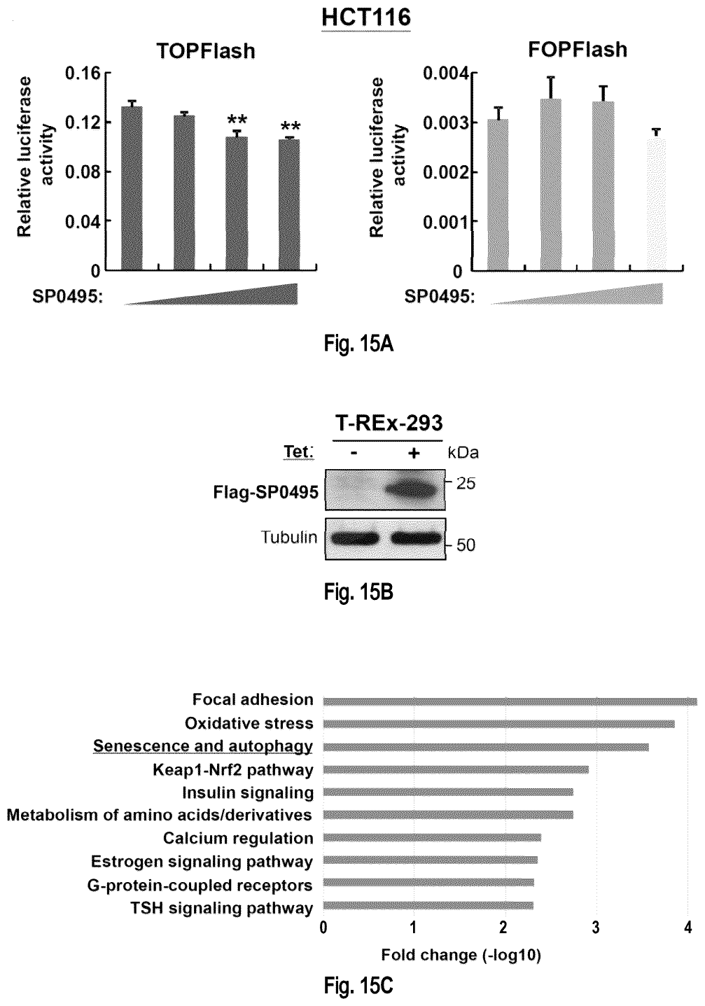

As SP0495 is a novel tumor suppressor located in the cytoplasm, we thus hypothesize that it might regulate cell signaling to exert its tumor suppression. We utilized several luciferase reporters of critical signaling pathways related to tumorigenesis, including p53-binding sites (bs) (p53), p21 promoter (p21), STATs-bs/GRR5 (STATs), NF-κB-bs (NF-κB), AP1-bs (INK), SRE (Ras/ERK) and TOPFlash (Wnt) pathways. It was found that the activities of p53 signaling reporters were significantly upregulated, but NF-kB and Wnt signaling reporters were significantly repressed by SP0495, in both HCT116 and KYSE150 cells (FIG. 5C), in addition to the repression of INK and STAT signaling reporters in KYSE150 only. Moreover, SP0495 suppressed TOPFlash reporter activity in a dose-dependent manner, while with no inhibitory effect on control FOPFlash reporter which harbors mutant TCF/LEF binding sites (FIG. 15A).

Furthermore, we examined the regulation of SP0495 on oncogenic signaling pathway regulators. We observed that phosphor-AKT at Ser473 (active form), phosphor-mTOR (Ser2448), phosphor-GSK3β at Ser9 (inactive form), and active β-catenin were downregulated by SP0495 expression (FIG. 5D). We also found that the active forms of NF-κB signaling molecules, including p-IKKα/β, p-IκBα, and p-p65, were suppressed in SP0495-expressing cells (FIG. 5D). Knockdown of SP0495 in turn increased the activities of these signaling pathways (FIG. 5E). Real-time PCR results showed that the expression of AKT-mTOR and NF-κB singling activators, PDK1, ID1, and EEF1A2, was downregulated by SP0495. Meanwhile, multiple downstream effectors of AKT/mTOR, Wnt, and NF-κB signaling, such as c-Myc, CCND1, MMP7, MITF, and TWIST1, were suppressed in SP0495-expressing cells compared to controls, although the mRNA level of β-catenin (CTNNB1) remained unchanged (FIG. 5F). We also confirmed the downregulation of Wnt signaling downstream effectors (c-Myc, CCND1, MMP7) at the protein level by SP0495 expression (FIG. 5G).

We then analyzed the relationship between co-expression of KIAA0495 RNA and signaling molecules in CRC using TCGA colorectal adenocarcinoma database. We found that KIAA0495 overexpression is associated with reduced expression of AKT/mTOR (EIF4EBP1 and RPS6KB1), NF-κB (BCL2, BCL2L1, BIRC5/Survivin, and SQSTM1/p62) and Wnt (ID1, CCND1, MYC and AP7) signaling molecules (FIG. 5H), with ID1 expression mutual exclusive with KIAA0495 expression (p=0.020). Co-expression analysis of Oncomine database showed that KIAA0495 was highly expressed in normal colon tissues, but with much lower expression in colorectal adenomas, while levels of Wnt signaling molecules negatively correlate with KIAA0495 levels across these samples (FIG. 5H). These results indicate that KIAA0495 (SP0495) contributes to the suppression of oncogenic signaling axis during tumor formation and progression.

### SP0495 Induces Autophagy in Carcinoma Cells

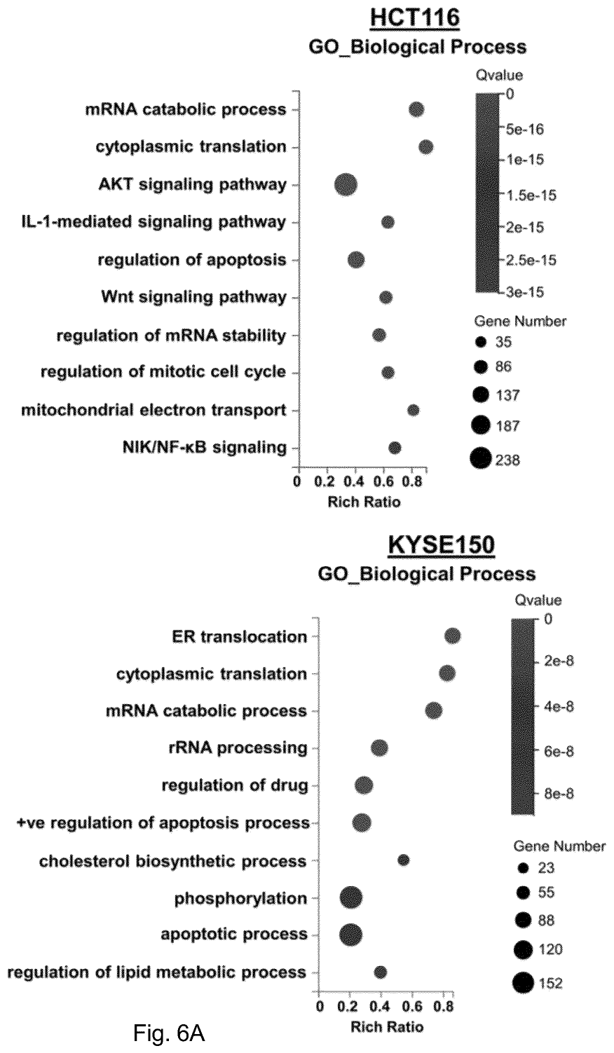

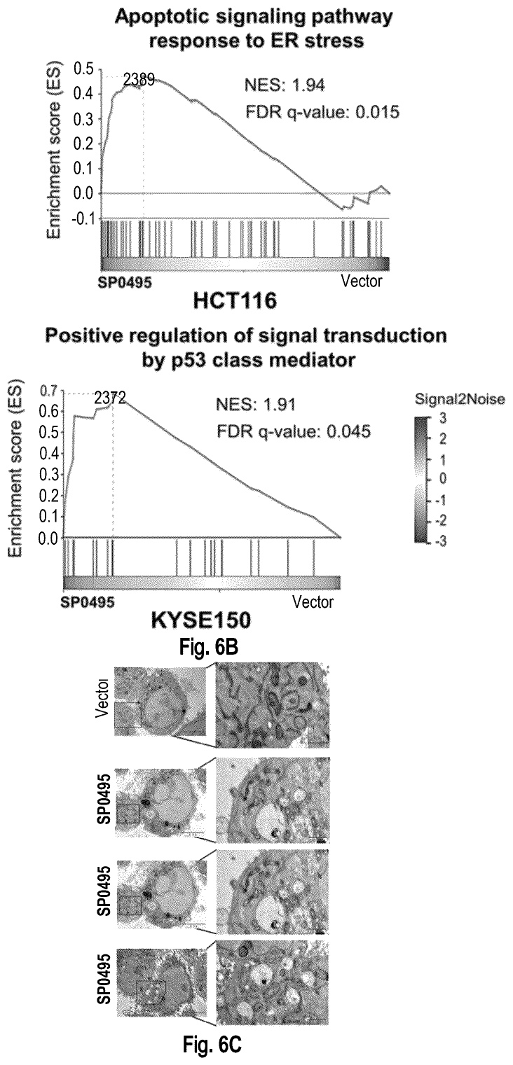

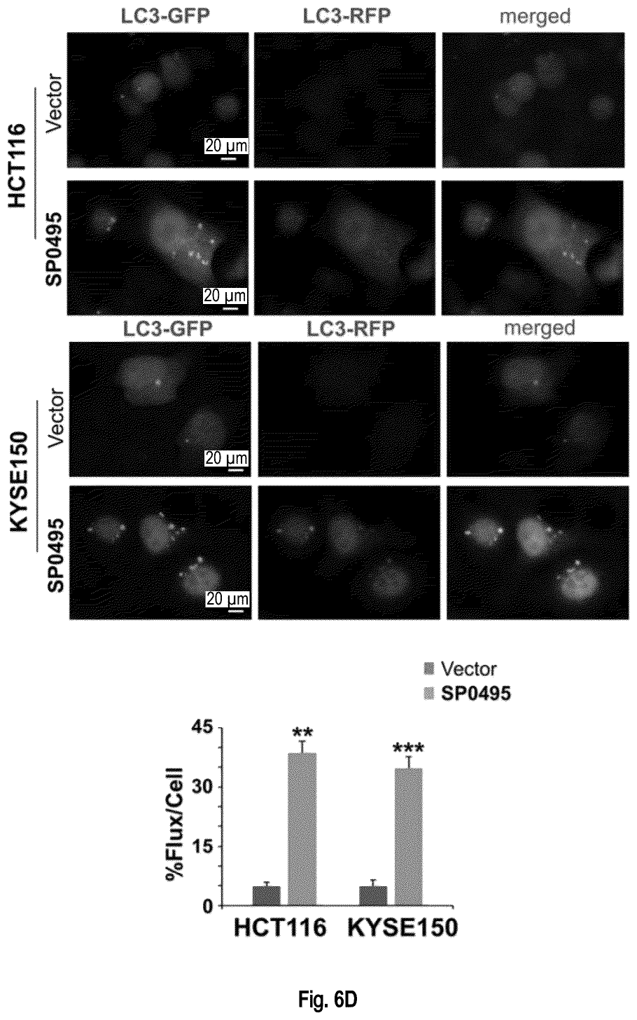

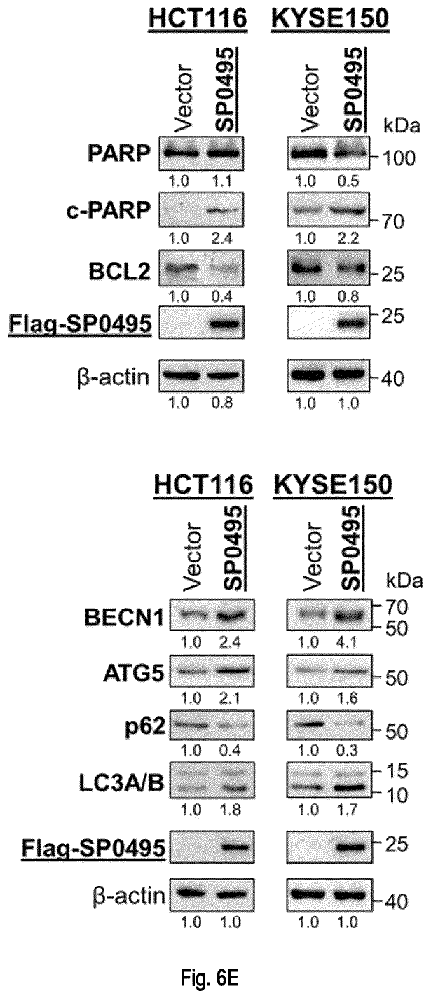

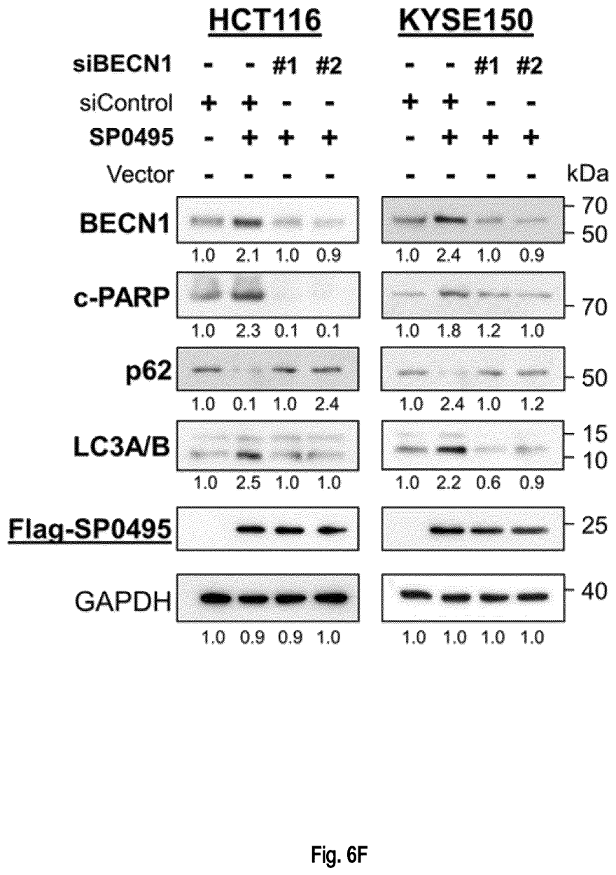

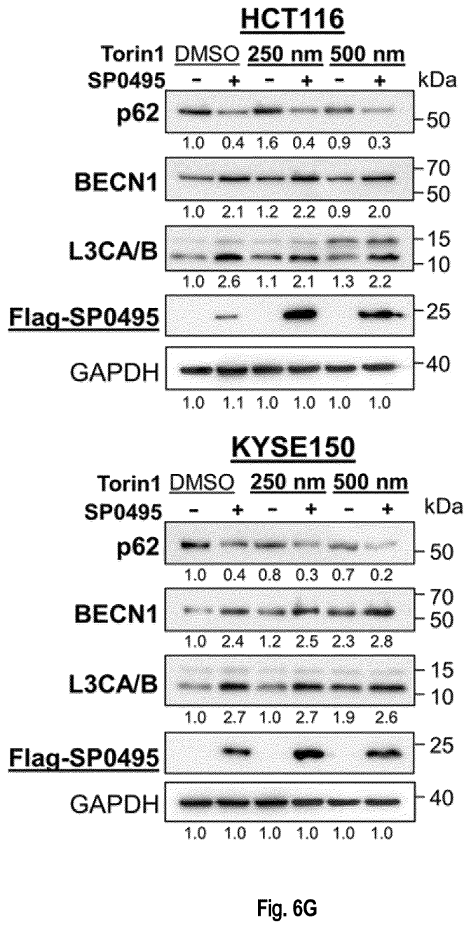

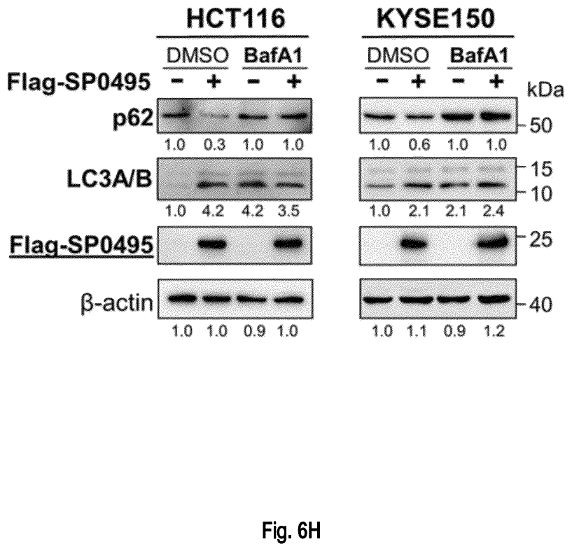

To further investigate the molecular mechanisms underlying SP0495 tumor suppression, we analyzed changes in gene expression profile mediated by SP0495 in tumor cells and immortalized normal cells through RNA-sequencing and microarray expression analysis. GO enrichment analysis showed that regulation of apoptosis and multiple oncogenic signaling pathways were the mainly enriched biological processes in HCT116 and KYSE150 cells (FIG. 6A). Microarray expression analysis showed that the enriched signaling pathways affected by doxycycline (DOX)-induced SP0495 expression in 293 cells included focal adhesion, senescence, and autophagy (FIG. 15B, C). Moreover, GSEA showed that SP0495 significantly induced apoptotic signaling response to ER stress and positively regulated the signal transduction by p53 mediators (FIG. 6B). These results are consistent with the suppression of autophagy-related oncogenic signaling (AKT/mTOR, NF-κB and Wnt) and activation of p53 signaling by SP0495 which we observed above in tumor cells.

We thus further examined the effects of SP0495 on autophagy in tumor cells. Transmission electron microscopy (TEM) showed increase in the formation of autophagic vesicles in SP0495-expressing HCT116 cells (FIG. 6C). Further autophagic flux using an mRFP-GFP-LC3 reporter construct showed that more ectopically expressed mRFP-GFP-LC3 was detected as red and yellow speckles in SP0495-expressing tumor cells than control cells, indicating that SP0495 induces accumulation of autophagosomes in tumor cells (FIG. 6D).

As SQSTM1/p62 and BECN1 levels are critical indicators of autophagic flux, we further detected autophagy regulators to confirm the regulation of SP0495 on autophagy. It was found that SP0495 upregulated the levels of cleaved-PARP, BECN1 and ATG5, while downregulated the levels of BCL2 and SQSTM1/p62, thus mediating autophagosome form LC3-II conversion in tumor cells (FIG. 6E). To demonstrate the role of BECN1 upregulation in SP0495-mediated autophagy, knockdown of BECN1 using siRNA was used in SP0495-expressing HCT116 and KYSE150 cells. Results showed that cleaved-PARP and L3CII protein levels were decreased after BECN1 knockdown but p62 levels were increased, indicating that BECN1 upregulation by SP0495 is responsible for its tumor-suppressive functions (FIG. 6F). We next examined the effects of SP0495 on Torin1-induced autophagy and found that SP0495 upregulated LC3II protein levels after Torin1 treatment, suggesting that SP0495-expressing tumor cells are more susceptible to cell death stimuli (FIG. 6G). Moreover, Bafilomycin A1 (BafA1), a lysosomal inhibitor, abrogated the autophagy induction by SP0495 in tumor cells (FIG. 6H). We further investigated whether the blockage of autophagic response in tumor cells preceded cell growth inhibition by SP0495. We found that inhibition of autophagic response with the lysosomal inhibitor BafA1 significantly enhanced SP0495-mediated inhibition of cell clonogenic ability, in both HCT116 and KYSE150 cells (FIG. 6I). These data indicate that SP0495 induces autophagic flux through regulating autophagy proteins in tumor cells.

### SP0495 Promotes Autophagy Through Enhancing BECN1 Stability

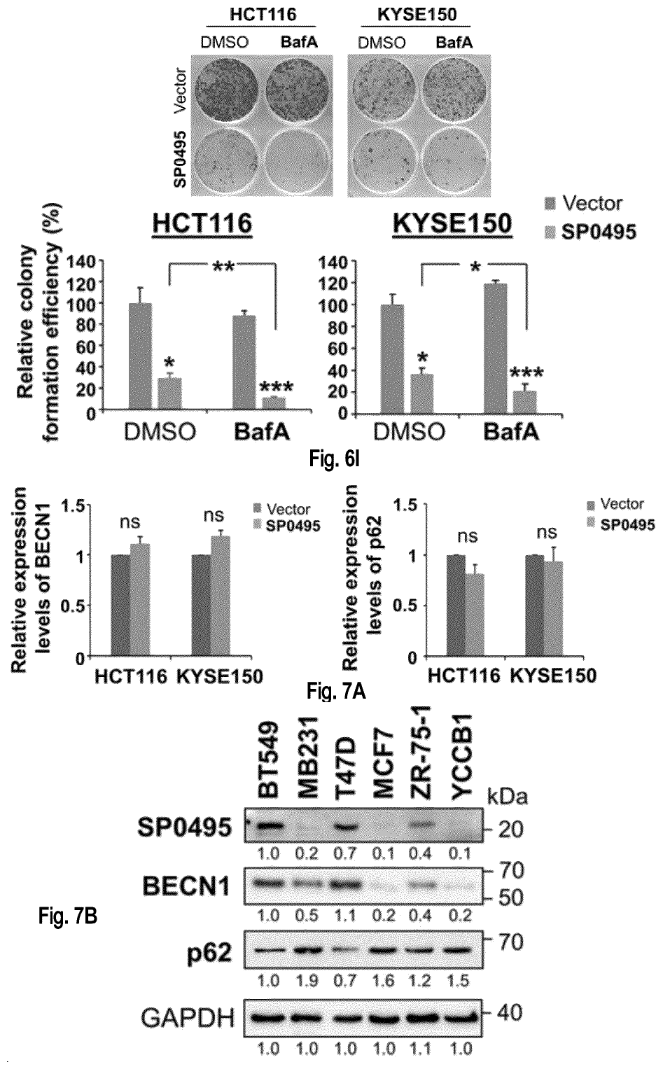

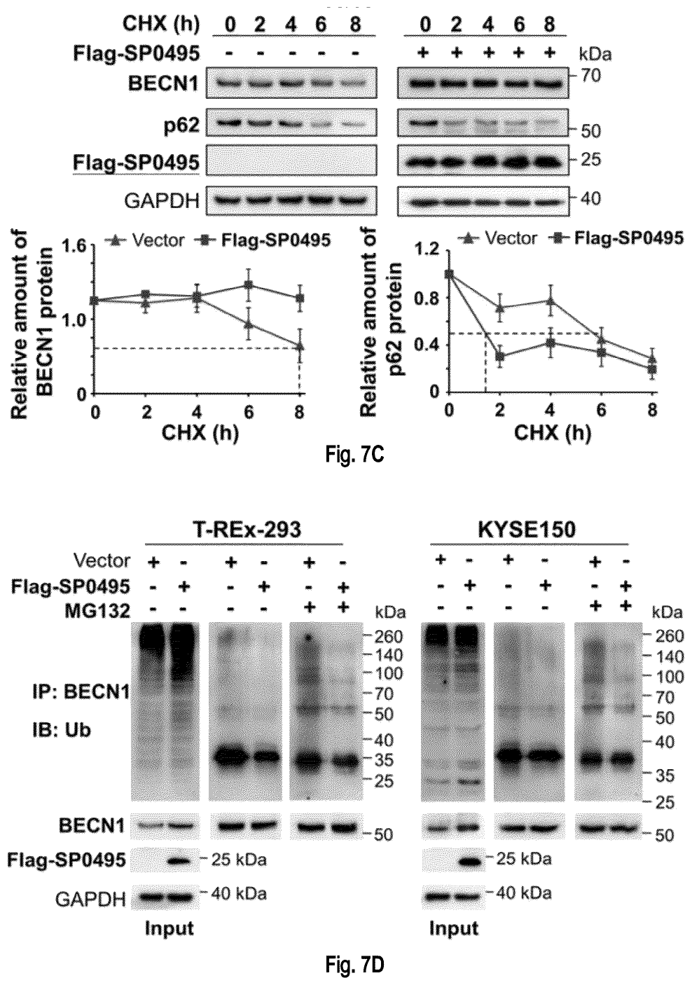

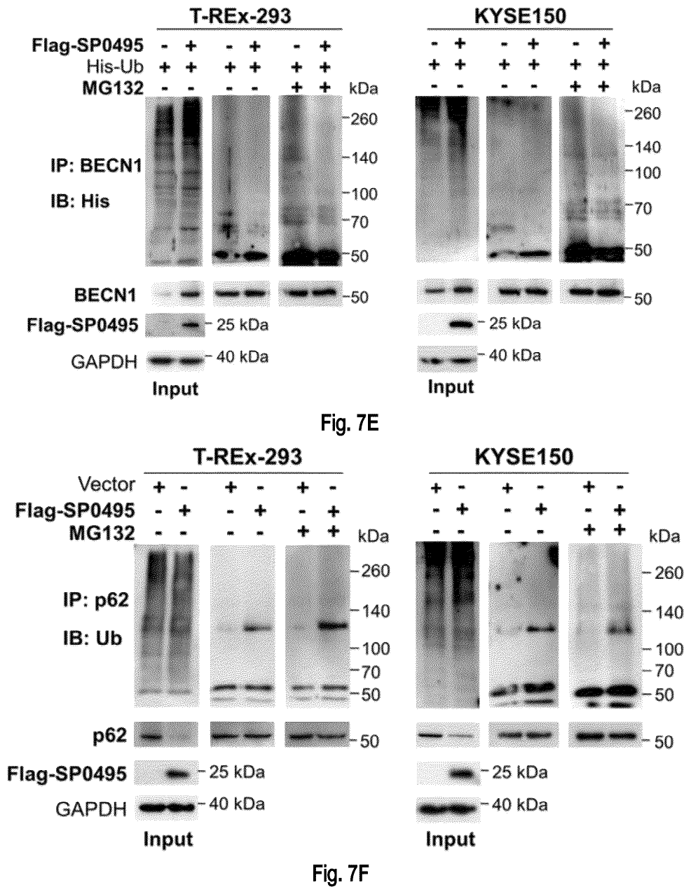

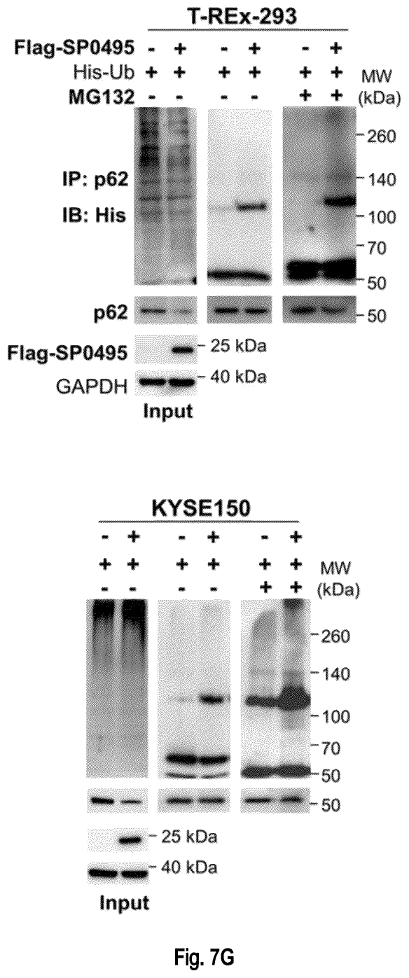

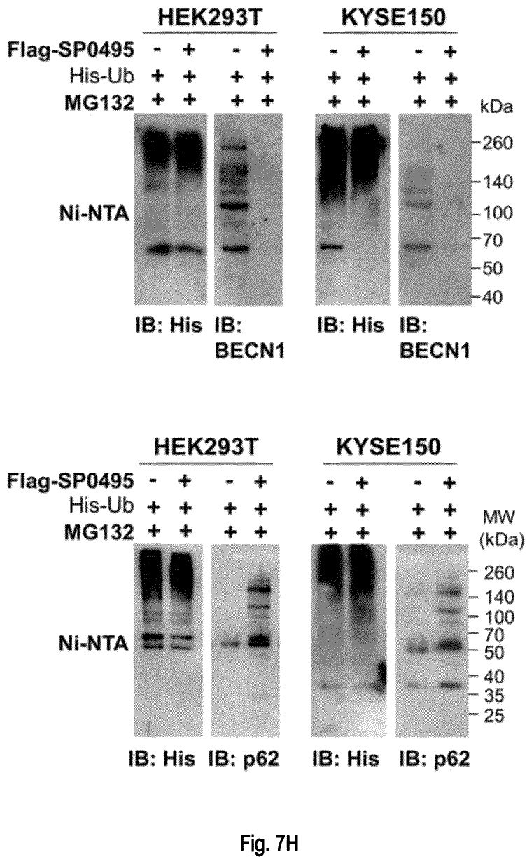

Autophagy is mainly regulated by BECN1 and SQSTM1/p62, with p62 as an autophagic degradation marker, we thus detected effects of SP0495 on BECN1 and p62 expression levels. Results showed that SP0495 had no significant effect on the mRNA expression levels of BECN1 and p62 (FIG. 7A), however endogenous SP0495 expression level is correlated with upregulated BECN1 level in a panel of cell lines (FIG. 7B), indicating that SP0495 might be involved in regulating BECN1 expression at the protein level.

As autophagy is a cellular degradation process through stabilizing BECN1 or degrading p62 via protein modifications such as ubiquitination, we next detected the half-life of endogenous BECN1 and p62 affected by SP0495 in tumor cells. CHX assay showed that SP0495 extended the half-life of BECN1 over 8 h, but shortened the half-life of p62 from 6 h to ˜2 h, indicating that SP0495 modulates the stability of both BECN1 and p62 proteins (FIG. 7C).

We next evaluated whether modulation of protein ubiquitination by SP0495 led to the regulation of BECN1 and p62 protein stabilities. To assess the effects of SP0495 on endogenous or exogenous ubiquitin chain of BECN1 and p62, we performed coimmunoprecipitation (co-IP) assays in SP0495-inducible expressed 293 cells and stably-expressed KYSE150 cells. We found that SP0495 decreased endogenous or exogenous ubiquitin linked with BECN1, in the presence and absence of proteasome inhibitor MG132 (FIG. 7D, E), while increased endogenous or exogenous ubiquitin linked with p62, in the presence and absence of MG132 (FIG. 7F, G). Furthermore, Ni-NTA (nickel nitrilotriacetic acid) ubiquitination assay showed decreased BECN1 ubiquitination but increased p62 ubiquitination by SP0495 in HEK293T and KYSE150 cells co-transfected with His-Ub and Flag-SP0495, after MG132 treatment (FIG. 7H). These results indicate that SP0495 regulates both BECN1 and p62 stability to induce autophagy in tumor cells.

### SP0495 Acts as a Lipid-Binding-Protein to Regulate AKT Signaling and Autophagy

To elucidate the underlying molecular mechanism of SP0495 in regulating the stabilities of BECN1 and p62 proteins, we examined the interaction of SP0495 with BECN1 or p62 by co-IP assay and observed no direct binding.

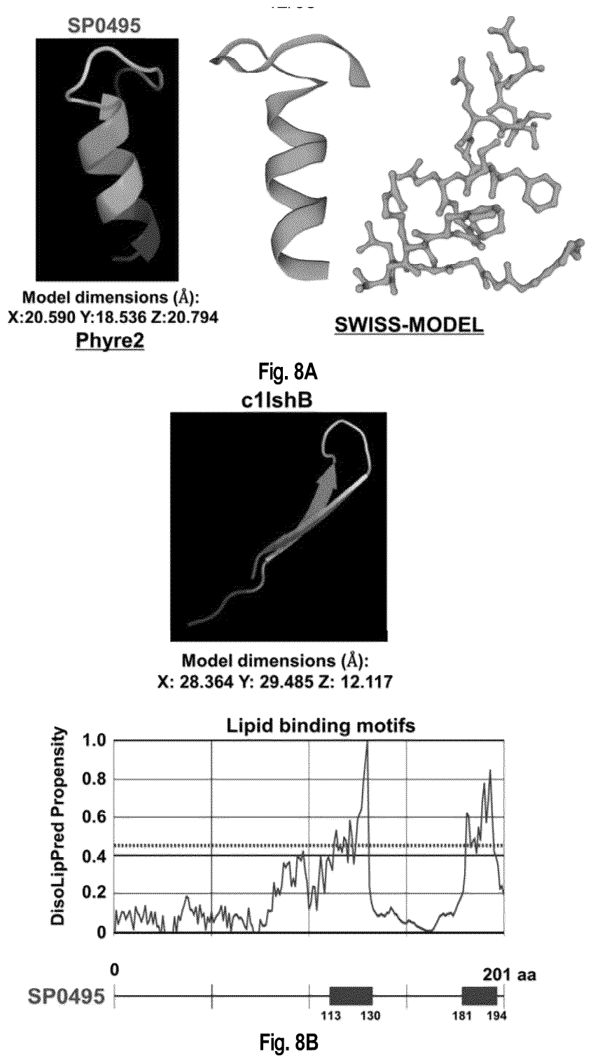

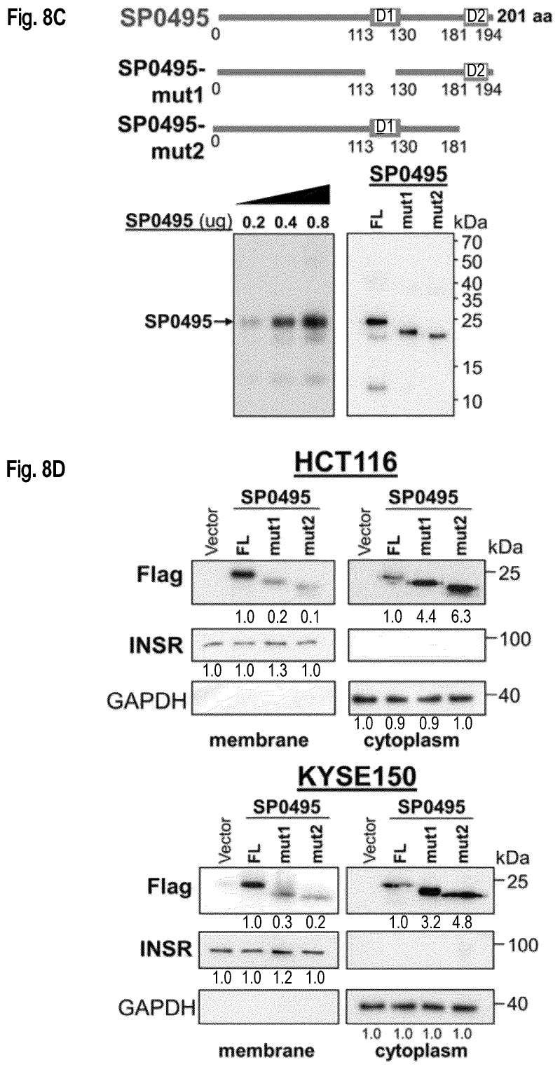

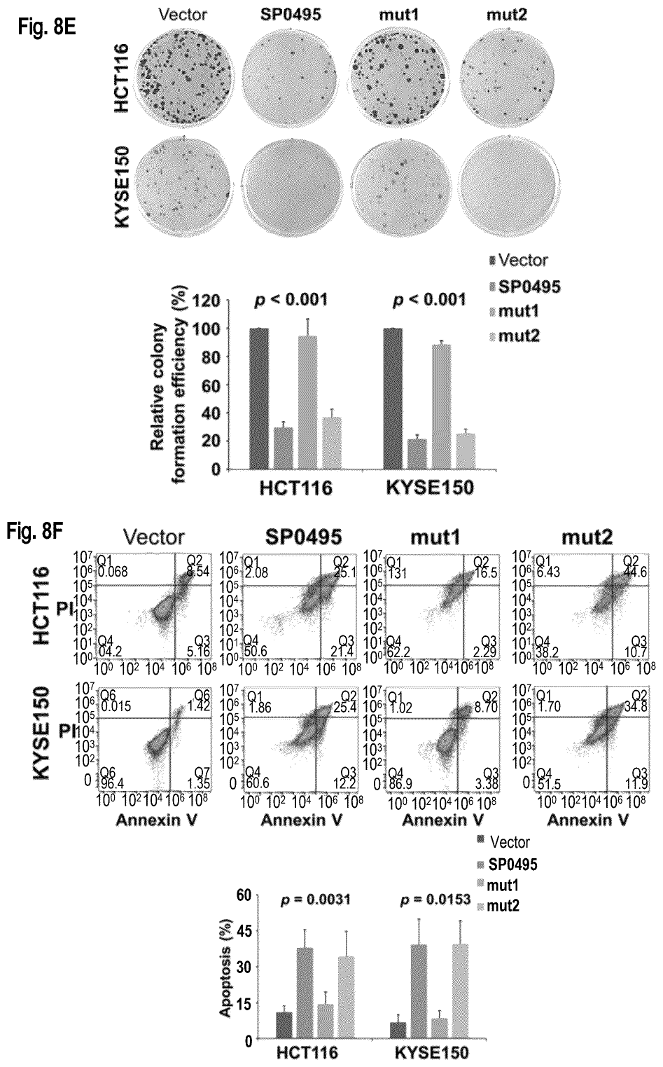

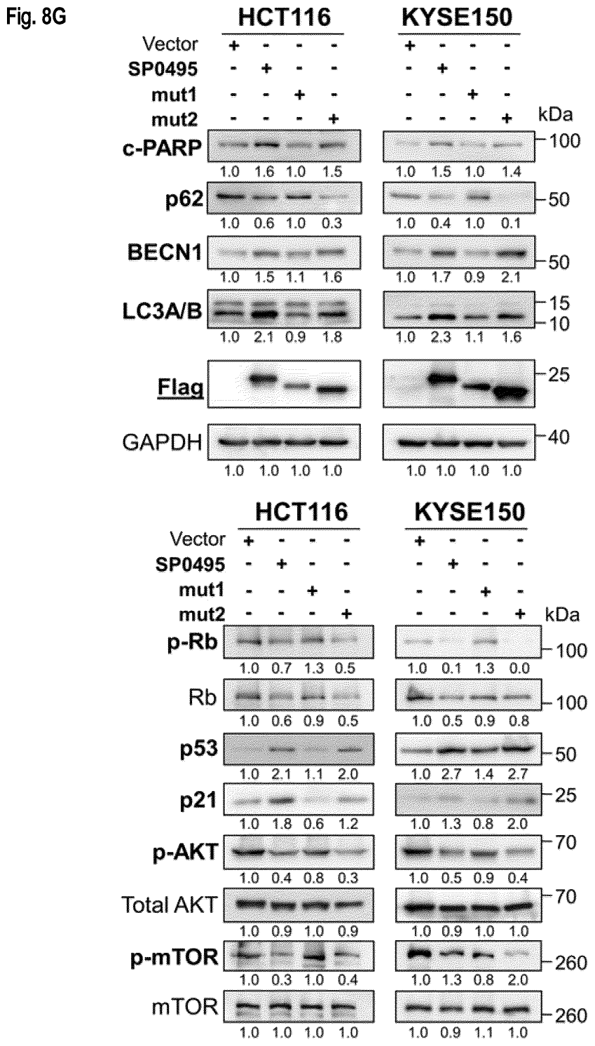

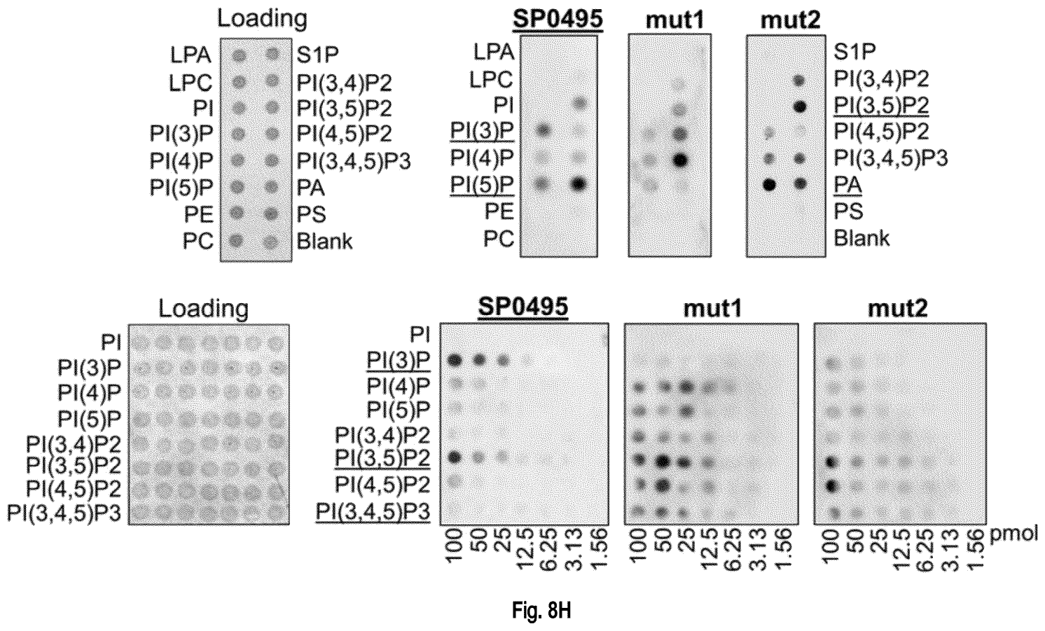

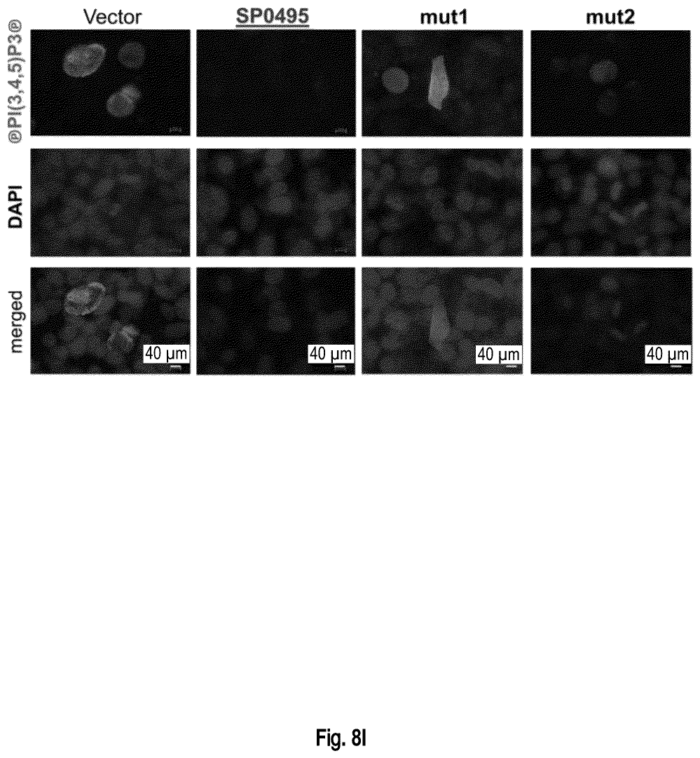

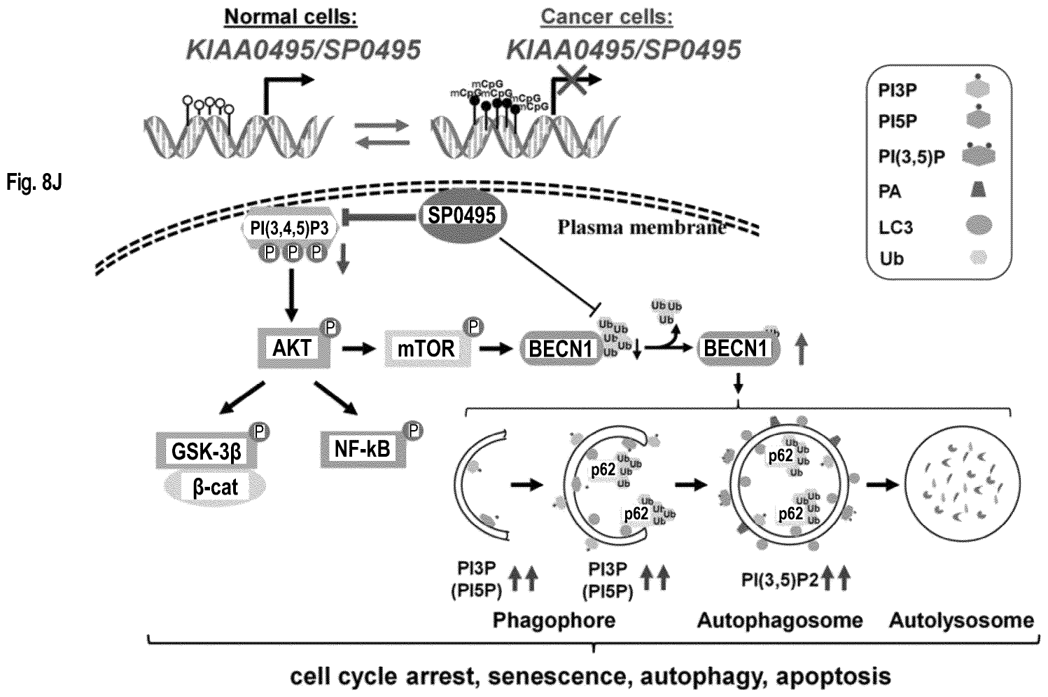

Autophagy is a membrane-driven catabolic pathway through the interaction of membrane lipids with autophagy machinery proteins. Phosphoinositides and phosphoinositide-binding proteins play essential roles in the regulation of lipid membrane trafficking/signaling, autophagy and cell signaling events, especially AKT activation and signaling³¹. Thus, we sought to investigate the possible interaction of SP0495 protein with phosphoinositides. First, we performed 3D structure model analysis of SP0495 protein by Phyre2 (FIG. 8A, left panel). The secondary structure of SP0495 was described as disordered of 53%, alpha-helix of 19%, and beta-strand of 17%. We also validated the predicted 3D structure of SP0495 by Swiss-Model (FIG. 8A, right panel). The MolProbity Score was 2.59 and Ramachandran favored 77.78% with QMEAN, Cβ, all-atom, solvation, and torsion values of −1.28, −0.83, −0.10, −0.89, and −1.01, respectively. The domain analysis by Phyre2 showed a 3D structure of lipid-binding-protein template (cllshB) out of SP0495 (FIG. 8B). Further protein motif analysis by DisoLipPred revealed two lipid-binding motifs within the SP0495 protein (FIG. 8B). These results indicate possible binding of SP0495 with phosphoinositides for the regulation of autophagy and cell signaling.

To assess that SP0495 functions as a lipid-binding-protein, we constructed two lipid-binding motif deletion mutants, SP0495-mutant 1 with lipid-binding domain 1 deleted and SP0495-mutant 2 with lipid-binding domain 2 deleted (FIG. 8C), with recombinant proteins of these SP0495 constructs successfully detected (FIG. 8C). We observed that the full-length SP0495 was mainly located on cell membrane but its mutants mainly located in the cytoplasm (FIG. 8D). Colony formation assay showed that mutant 1 lost the ability of cell growth inhibition and apoptosis induction, while the ability of cell growth inhibition and apoptosis induction of mutant 2 was only slightly decreased or not affected at all (FIG. 8E, F). We further examined the effects of SP0495 mutants on cell signaling and autophagy marker expression. Results showed that SP0495-mutant 1 was seriously impaired in the ability to activate p53 signaling, suppress AKT/mTOR signaling and Rb phosphorylation, and autophagic signaling induction (FIG. 8G). These results indicate that the lipid-binding domain 1 is a major and critical functional domain for SP0495-mediated tumor-suppressive functions.

We further investigated the possible interaction of phosphoinositide with SP0495. Lipid overlay experiments using PIP strips were performed using recombinant human SP0495 protein and its two mutants (FIG. 8C, D). We found that human SP0495 protein mainly interacted with phosphatidic acid (PA), PtdIns(3)P, PtdIns(5)P and PtdIns(3, 5)P2, with a preference for PA, weakly with PtdIns(3,4,5)P3, PtdIns(4, 5)P2 and PtdIns(4)P (FIG. 8H), while mutant1 primarily interacted with PI(3, 4,5)P3 and PI (4, 5)P2, but mutant 2 remained similar binding feature as the wild-type SP0495 protein. Protein-lipid binding assay using PIP arrays also revealed that SP0495 strongly interacted with PI(3)P and PI(3,5)P2, while mutant1 displayed almost no PI3P interaction (FIG. 8H). We further performed immunofluorescence staining to examine the expression levels of PI(3,4,5)P3 in tumor cells expressing SP0495 and its mutants. Results show that anti-PI(3,4,5)P3 staining was diminished or greatly reduced in tumor cells expressing SP0495 or mutant 2, while expression of mutant 1 did not affect PI(3,4,5)P3 staining much (FIG. 8I). These results indicate that SP0495, indeed as a lipid-binding protein, regulates autophagy and cell signaling through interacting with phosphoinositides in cells.

### Discussion

1p36.3 is an important TSG locus implicated in the early events of tumorigenesis in multiple cancers and is thus believed to harbor critical TSGs^{1, 2}. Although several TSGs residing in this locus, including TP73⁴, CHD5⁵, PRDM16⁶ and AJAP1⁷, have already been validated, more TSG candidates are likely still waiting to be characterized as 1p36.3 is a gene-rich region. In this report, through integrative epigenome study, we identified a novel 1p36.3 gene, KIAA0495, frequently methylated and silenced in broad cancers in a tumor-specific manner, indicating its tumorigenesis-associated functions. We further present direct evidence that although KIAA0495 was previously claimed to be a lncRNA, it actually encodes a small protein SP0495, which induces tumor cell apoptosis, cell cycle arrest, senescence, autophagy, and inhibits tumor cell growth in vivo. SP0495 represses oncogenic AKT/mTOR, NF-κB, and Wnt/β-catenin signaling. As a lipid-binding protein, SP0495 regulates autophagy through disrupting autophagic/proteasomal degradation of p62 and BECN1. We also found that, although KIAA0495 transcript/protein is broadly expressed in multiple normal tissues, it is silenced in multiple tumors by promoter CpG methylation, and its downregulation/methylation is associated with high-grade stage and poor survival of multiple cancer patients. Thus, our results validate that KIAA0495/SP0495 is a bonafide TSG/tumor suppressor being frequently inactivated by epigenetic mechanisms in multiple cancers.

Genome-wide association studies (GWAS) have pinpointed 1p36 as a susceptibility locus for multiple cancers^{32, 33, 34} and even certain developmental disorders such as azoospermia³⁵. Other 1p36.3 TSGs have previously been reported to contribute to multiple tumorigeneses. For example, TP73 is mapped to a minimal region of 1p36.3 commonly deleted in neuroblastomas, and functions as a p53-like TSG, inducing cell cycle arrest and apoptosis. Epigenetic silencing of TP73 leads to cell cycle deregulation in hematological and oligodendroglial tumors^{4, 36}. Through mouse chromosome engineering, Bagchi et al. identified a critical 1p36.31 tumor suppressor—the chromodomain helicase DNA binding domain 5 (CHD5), which controls cell proliferation, apoptosis, and senescence via p14^{ARF}/p53 pathway⁵, and is also epigenetically silenced in multiple cancers^{37, 38, 39, 40} Thus, novel 1p36 TSGs may be important for cancers and other diseases associated with genetic loss of this small genome fragment or epigenetic inactivation of its encoded genes.

A long 1p36.3 transcript KIAA0495 was first submitted to the NCBI database by Kazusa DNA Research Institute in 1997⁴¹. In the current NCBI database, this gene is named as TP73-AS1, as this gene is in the immediate vicinity of TP73 and the KIAA0495 transcript is partially complementary to the TP73 transcript. However, the complement is only at a small region (˜218 bp) of the C terminal of the TP73 gene, thus the previously named term TP73-AS1 is actually somewhat misleading (FIG. 10). A previous report indicated that full-length KIAA0495 cDNA (˜6.4 kb) may not produce protein by in vitro translation analysis⁴². However, the authors did observe a protein product (˜24 kDa) when expressing the ORF2 of KIAA0495 (606 nucleotides, 201 aa residues) using in-house made anti-PDAM/KIAA0495 antibody (a peptide TTSDLSAREDATPSC against 66-79 amino acid residues)⁴², which is consistent with our findings regarding the coding potential of KIAA0495-ORF2 (SP0495).

Recently with the advancement of biological technology, it has been recognized that some non-canonical ORFs derived from lncRNAs or UTRs do possess peptide/small protein-coding properties^{12, 13, 43, 44, 45}. Some of these peptide/small proteins have been validated as functional oncogenes or TSGs in tumorigenesis^{18, 19, 45}. Here, we demonstrate that the KIAA0495-ORF2 codes a small protein SP0495, through in vitro translation and further endogenous protein detection in cell lines and normal tissues, by Western blot and immunostaining using an antibody targeting the SP0495 protein. KIAA0495/SP0495 is readily expressed in multiple normal tissues, although with various expression levels. However, it is frequently downregulated, but rarely mutated, in multiple tumors including esophageal, colorectal, gastric, and breast cancers, and correlated with poor survival of multiple cancer patients, indicating its important roles in cancer pathogenesis.

As SP0495 protein is a previously uncharacterized protein, we investigated its biological functions and underlying mechanisms in-depth. SP0495 contains a transmembrane and signal peptide domain, and is located in the cytoplasm and partly co-localized with ER and Golgi. SP0495 exerts tumor-suppressive functions through inhibiting proliferation, inducing apoptosis and G1/S cell cycle arrest. Moreover, SP0495 suppresses tumor growth in vivo, thus indeed as a bonafide tumor suppressor. Mechanistically, SP0495 negatively regulates AKT/mTOR, NF-κB, and Wnt/β-catenin signaling cascades, through repressing AKT/mTOR and NF-kB upstream effectors including PDK1/PDPK1⁴⁶, D1⁴⁷, EEF1A2⁴⁸, and downstream targets, although PI3K remains unaffected. Genome-wide dataset analysis revealed that KIAA0495 is unregulated ˜1.75 fold in tumor cells which are sensitive to PI3K/AKT inhibitor (GDC-0941), compared to resistant cells 49, indicating that KIAA0495 inactivation may confer tumor cells resistance to such targeted therapy. Furthermore, SP0495-regulated genes by RNA-seq and microarray analysis are mainly enriched in signaling pathways of apoptotic regulation and p53 signaling, which is consistent with deregulation of AKT/mTOR, Wnt/β-catenin and NF-κB signaling cascades by SP0495.

We further found that SP0495 expression enhances apoptosis, cell cycle G1/S arrest, cell senescence and autophagy, by inducing elevated protein levels of p53, phosphorylated p53 at Ser15 and p21, which confirmed our RNA-seq and microarray data. In cancers, genetic/epigenetic aberrations of autophagy regulators highlight the importance of autophagy dysregulation in cancer pathogenesis and even drug resistance. Multiple tumor suppressors have been identified to induce autophagy, including BECN1, p53, PTEN, DAPK1 and LKB1/STK11⁵⁰. Autophagy regulators p62 and BECN1 are inversely correlated and play crucial roles in autophagy regulation in cells.

Our study demonstrates that SP0495 as a functional tumor suppressor induces autophagy in tumor cells. SP0495 promotes BECN1 accumulation but inhibits p62 accumulation, through interfering with their ubiquitination-related degradation, then further promoting autophagy. We further found that knockdown of BECN1 in SP0495-expressing tumor cells impaired its induced apoptosis and autophagy, indicating a key role of BNEC1 in SP0495-mediated tumor suppression. However, unlike other regulators, SP0495 does not bind p62 and BECN1 directly. SP0495 is speculated to regulate autophagy as a transmembrane signal peptide, since autophagy is a membrane-driven process with lipids playing a central role in its regulation. Phosphoinositides and phosphoinositide-binding proteins play essential roles in the regulation of lipid membrane trafficking/signaling, autophagy and cell signaling events, especially AKT activation and signaling³¹. AKT needs to bind to PI(3,4,5)P3 on the plasma membrane inner leaflet via its PH domain, then undergoes conformational changes, phosphorylation/activation and further downstream signaling cascade.

However, how lipids interact with autophagy machinery regulators by SP0495 still remains unclear. Our structure analysis shows that SP0495 has similarities with lipid-binding proteins and contains two lipid-binding domains, indicating that SP0495 possibly regulates autophagy and AKT signaling as a lipid-binding protein. Phosphoinositides (PtdIns; phosphorylated derivatives of PI), consisting of ˜1% of phospholipids, play key roles in lipid signaling and membrane trafficking pathways including autophagy. Emerging evidence demonstrates the important role of PI(5)P in positively regulating autophagy, through association with autophagy effectors that bind PI(3)P⁵¹. PI(5)P and PI(3,5)P2 are mainly localized at lysosome and autophagosome within the intracellular membrane system^{52, 53}. Moreover, phosphatidic acid (PA) acts as a positive regulator of autophagy via inhibiting mTORC1⁵⁴. Our data show that SP0495 interacts with distinct types of lipids by protein-lipid overlay assays, including PA, PI(3)P, PI(5)P and PI(3,5)P2, which facilitates the biogenesis and maturation of autophagosomes. Moreover, SP0495 predominantly binds PI(3)P and PI(3,5)P2, supporting our hypothesis that SP0495 regulates autophagy and AKT signaling through binding phosphoinositides. Future studies like the liposome flotation assay might verify these hypothesized protein-phosphoinositide interactions and identify the functional domain or residues of SP0495 involved in the interaction.

We also assessed the regulatory mechanisms of KIAA0495 downregulation in cancers. Promoter CpG methylation mediates KIAA0495 silencing/downregulation in multiple tumors, but rarely in normal tissues and immortalized normal cells. Moreover, no mutations were detected in any of the tumor cell lines examined, indicating a predominant role for epigenetic inactivation of KIAA0495 in multiple cancers. We also found that KIAA0495 promoter methylation is significantly associated with poor survival of rectum, esophageal, and breast cancers, thus could be an attractive epigenetic biomarker for tumor diagnosis in future.

In conclusion, our integrative epigenomic analysis elucidates a new molecular link of a novel 1p36 tumor suppressor to multiple tumorigeneses. The small protein SP0495, encoded by the pre-claimed lncRNA KIAA0495 (TP73-AS1), functions as a bonafide tumor suppressor for multiple cancers through suppressing oncogenic signaling and regulating cell cycle, apoptosis, senescence and autophagy (FIG. 8J). The discovered tumor-specific methylation of KIAA0495 promoter could serve as a potential tumor biomarker in future.

### Methods

### CpG Methylomes

CpG methylome analysis of cell lines was performed by methylated DNA immunoprecipitation (MeDIP) coupled with promoter microarray hybridization (MeDIP-chip)⁵⁵. Briefly, genomic DNA of CRC cell lines (HCT116-DKO, HCT116), gastric cell line (SNU719), NPC cell line (C666-1), breast cancer cell lines (MB231, MCF7), and immortalized mammary epithelial cell line (HMEpC) was immunoprecipitated using a monoclonal antibody against 5-methylcytidine (33D3, Diagenode, Seraing, Belgium), purified, labeled and hybridized to NimbleGen™ HG18 Meth (385K CGI plus) promoter arrays (Array Star, Inc., MD). Array data analysis of methylome data was performed using SignalMap by NimbleGen Systems, Inc. as previously described⁵⁵.

### In Vitro Translation of KIAA0495-ORF2 Protein

In vitro protein expression was performed using the Human In Vitro Protein Expression Kit for DNA Templates (Thermo Fisher Scientific, Rockford, IL) according to the manufacturer's instructions. Protein products were analyzed in SDS-PAGE gel and immunoblot with antibodies against FLAG tag (F3165, Sigma) or KIAA0495-ORF2/SP0495 (TA503634, Origene).

### In Vivo Xenograft Models

Female BALB/c nude mice aged 4 weeks were used for tumor implantation experiments. HCT116 cells with luciferase-tag (2-5×10⁶ cells in PBS) were injected subcutaneously into the flanks of nude mice with randomization (n=6). No blinding to the group allocation during the experiment was done. Starting on day 10 after the first injection, tumor growth was monitored once every 7-10 days for 40 days according to the actual tumor formation and animal welfare ethics regulations (tumor diameter <20 mm). For luciferase luminescent detection, a dose of 10 ul/g D Luciferin (15 mg/mL) was injected intraperitoneally before anesthesia testing. Tumor images were captured using an IVIS® Lumina LT for whole live-animal imaging (PerkinElmer). Total fluorescence expression in the AVERAGE area was calculated as ([p/s]/[μW/cm2]). Tumor volume was calculated as [π/6×L (length)×W (width)×H (height)]. All animal work was approved by the Institutional Ethics Committees of the First Affiliated Hospital of Chongqing Medical University.

### Autophagic Flux and Quantification

To measure autophagic flux, we used monomeric LC3 proteins fused to a pH-stable RFP and a pH-sensitive GFP fluorophore. The mRFP-GFP-LC3 adenoviral particles were purchased from HanBio Technology (Shanghai, China). HCT116 and KYSE150 cells were infected with adenoviral particles according to the manufacturer's instructions. After infection, cells were cultured for another 24 h for immunostaining. RFP punctate indicates both early autophagosome and autophagic lysosomes. Yellow punctate appearing after red and green fluorescence merged indicates early autophagosomes alone, as GFP fluorescence is quenched when autophagosomes fuse with lysosomes. The ratio of early autophagosomes over total autolysosomes was calculated as Flux %: Flux %=(100−((Red and Green)/Red)×100). Images were captured by a fluorescence microscope Olympus BX51 microscope (Olympus Corporation, Tokyo, Japan).

### Protein Ubiquitination Assay

Protein ubiquitination assay was performed as described previously^{56, 57, 58} Briefly, T-REx-293 cells with inducible SP0495 expression and KYSE150 cells with stable expression of SP0495 were lysed, or transfected with His-Ub plasmids for 48 hours. After cell treatment with 10 μM MG132 (Sigma-Aldrich, Saint Louis, MO) for 6 h before harvest, endogenous BECN1 and p62 were immunoprecipitated with BECN1 and p62 antibodies, followed by immunoblot with anti-Ub or anti-His antibody to detect the ubiquitinated BECN1 and p62 proteins.

Ni-NTA pull-down assay was performed as previously described⁵⁶. Cell lysates were affinity purified with Ni-NTA-agarose beads (#30210, Qiagen), and analyzed by immunoblot with specific antibodies targeting BECN1 and p62.

### Lipid-Protein Overlay Assay

PIP Strips™ and PIP Arrays™ (Echelon Biosciences) were blocked in TBST containing 3% BSA for 1 hr at room temperature (RT) and incubated overnight at 4° C. with 0.5 μg/ml recombinant KIAA0495-ORF2/SP0495 protein (Origene, TP310801) and customized SP0495-mutant 1 and mutant 2 recombinant proteins produced with C-terminal DDK tag from human HEK293 cells (Origene) in TBST+3% BSA. After three times washes, anti-KIAA0495-ORF2/SP0495 antibody (Origene, TA503634) was added to TBST+3% BSA solution and incubated for 1 hr at RT. Bound proteins were then detected using an HRP-coupled anti-mouse-IgG antibody, followed by visualization using the ECL detection system (GE Healthcare).

### Additional Materials and Methods

Additional information on cell lines, tumor and normal tissue samples, array-comparative genomic hybridization (CGH), semi-quantitative RT-PCR and real-time PCR analyses, bisulfite treatment and promoter methylation analyses, plasmid constructs and generation of cell lines, immunofluorescence, immunohistochemical staining, colony formation assay, in vivo tumor formation assay, apoptosis and cell cycle analyses, Senescence-specific β-galactosidase staining, luciferase reporter assay, transmission electron microscopy, Western blot, protein stability analysis, generation of microarray data and online URLs, and statistical analyses are provided in Supplementary information.

### REFERENCES

1. Henrich K O, Schwab M, Westermann F. 1p36 tumor suppression—a matter of dosage? Cancer Res 2012, 72(23): 6079-6088.
2. Bagchi A, Mills A A. The quest for the 1p36 tumor suppressor. Cancer Res 2008, 68(8): 2551-2556.
3. Solimini N L, Xu Q, Mermel C H, Liang A C, Schlabach M R, Luo J, et al. Recurrent hemizygous deletions in cancers may optimize proliferative potential. Science 2012, 337(6090):104-109.
4. Kawano S, Miller C W, Gombart A F, Bartram C R, Matsuo Y, Asou H, et al. Loss of p73 gene expression in leukemias/lymphomas due to hypermethylation. Blood 1999, 94(3): 1113-1120.
5. Bagchi A, Papazoglu C, Wu Y, Capurso D, Brodt M, Francis D, et al. CHD5 is a tumor suppressor at human 1p36. Cell 2007, 128(3): 459-475.
6. Xinh P T, Tri N K, Nagao H, Nakazato H, Taketazu F, Fujisawa S, et al. Breakpoints at 1p36.3 in three MDS/AML(M4) patients with t(1;3)(p36;q21) occur in the first intron and in the 5′ region of MEL1. Genes Chromosomes Cancer 2003, 36(3): 313-316.
7. Lin N, Di C, Bortoff K, Fu J, Truszkowski P, Killela P, et al. Deletion or epigenetic silencing of AJAP1 on 1p36 in glioblastoma. Mol Cancer Res 2012, 10(2): 208-217.
8. Ingolia N T, Brar G A, Stern-Ginossar N, Harris M S, Talhouarne G J, Jackson S E, et al. Ribosome profiling reveals pervasive translation outside of annotated protein-coding genes. Cell Rep 2014, 8(5): 1365-1379.
9. Fields A P, Rodriguez E H, Jovanovic M, Stern-Ginossar N, Haas B J, Mertins P, et al. A Regression-Based Analysis of Ribosome-Profiling Data Reveals a Conserved Complexity to Mammalian Translation. Mol Cell 2015, 60(5): 816-827.
10. Calviello L, Mukherjee N, Wyler E, Zauber H, Hirsekorn A, Selbach M, et al. Detecting actively translated open reading frames in ribosome profiling data. Nat Methods 2016, 13(2): 165-170.
11. Chen J, Brunner A D, Cogan J Z, Nunez J K, Fields A P, Adamson B, et al. Pervasive functional translation of noncanonical human open reading frames. Science 2020, 367(6482): 1140-1146.
12. Magny E G, Pueyo J I, Pearl F M, Cespedes M A, Niven J E, Bishop S A, et al. Conserved regulation of cardiac calcium uptake by peptides encoded in small open reading frames. Science 2013, 341(6150): 1116-1120.
13. Andrews S J, Rothnagel J A. Emerging evidence for functional peptides encoded by short open reading frames. Nat Rev Genet 2014, 15(3): 193-204.
14. Prensner J R, Enache O M, Luria V, Krug K, Clauser K R, Dempster J M, et al. Noncanonical open reading frames encode functional proteins essential for cancer cell survival. Nat Biotechnol 2021, 39(6): 697-704.
15. Huang J Z, Chen M, Chen, Gao X C, Zhu S, Huang H, et al. A Peptide Encoded by a Putative lncRNA HOXB-AS3 Suppresses Colon Cancer Growth. Mol Cell 2017, 68(1): 171-184 e176.
16. Meng N, Chen M, Chen, Chen X H, Wang J Z, Zhu S, et al. Small Protein Hidden in lncRNA LOC90024 Promotes “Cancerous” RNA Splicing and Tumorigenesis. Adv Sci (Weinh) 2020, 7(10): 1903233.
17. Zhu S, Wang J Z, Chen, He Y T, Meng N, Chen M, et al. An oncopeptide regulates m(6)A recognition by the m(6)A reader IGF2BP1 and tumorigenesis. Nat Commun 2020, 11(1): 1685.
18. Zhang M, Zhao K, Xu X, Yang Y, Yan S, Wei P, et al. A peptide encoded by circular form of LINC-PINT suppresses oncogenic transcriptional elongation in glioblastoma. Nat Commun 2018, 9(1): 4475.
19. Wang Y, Wu S, Zhu X, Zhang L, Deng J, Li F, et al. LncRNA-encoded polypeptide ASRPS inhibits triple-negative breast cancer angiogenesis. J Exp Med 2020, 217(3).
20. Mathew R, Karantza-Wadsworth V, White E. Role of autophagy in cancer. Nat Re Cancer 2007, 7(12): 961-967.
21. Santana-Codina N, Mancias J D, Kimmelman A C. The Role of Autophagy in Cancer. Annu Rev Cancer Biol 2017, 1: 19-39.
22. Tyutyunyk-Massey L, Gewirtz D A. Roles of autophagy in breast cancer treatment: Target, bystander or benefactor. Semin Cancer Biol 2020, 66: 155-162.
23. Tang J, Deng R, Luo R Z, Shen G P, Cai M Y, Du Z M, et al. Low expression of ULK1 is associated with operable breast cancer progression and is an adverse prognostic marker of survival for patients. Breast Cancer Res Treat 2012, 134(2): 549-560.
24. Li Z L, Zhang H L, Huang Y, Huang J H, Sun P, Zhou N N, et al. Autophagy deficiency promotes triple-negative breast cancer resistance to T cell-mediated cytotoxicity by blocking tenascin-C degradation. Nat Commun 2020, 11(1): 3806.
25. Li Z, Chen B, Wu Y, Jin F, Xia Y, Liu X. Genetic and epigenetic silencing of the beclin 1 gene in sporadic breast tumors. BMC Cancer 2010, 10: 98.
26. Rolland P, Madjd Z, Durrant L, Ellis I O, Layfield R, Spendlove I. The ubiquitin-binding protein p62 is expressed in breast cancers showing features of aggressive disease. Endocr Relat Cancer 2007, 14(1): 73-80.
27. Luo R Z, Yuan Z Y, Li M, Xi S Y, Fu J, He J. Accumulation of p62 is associated with poor prognosis in patients with triple-negative breast cancer. Onco Targets Ther 2013, 6: 883-888.
28. Lu Z, Luo R Z, Lu Y, Zhang X, Yu Q, Khare S, et al. The tumor suppressor gene ARHI regulates autophagy and tumor dormancy in human ovarian cancer cells. J Clin Invest 2008, 118(12): 3917-3929.
29. Ying J, Li H, Seng T J, Langford C, Srivastava G, Tsao S W, et al. Functional epigenetics identifies a protocadherin PCDH10 as a candidate tumor suppressor for nasopharyngeal, esophageal and multiple other carcinomas with frequent methylation. Oncogene 2006, 25(7): 1070-1080.
30. Jin H, Wang X, Ying J, Wong A H, Cui Y, Srivastava G, et al. Epigenetic silencing of a Ca(2+)-regulated Ras GTPase-activating protein RASAL defines a new mechanism of Ras activation in human cancers. Proc Natl Acad Sci USA 2007, 104(30): 12353-12358.
31. Kim S, Heo S, Brzostowski J, Kang D. Endosomal mTORC2 Is Required for Phosphoinositide-Dependent AKT Activation in Platelet-Derived Growth Factor-Stimulated Glioma Cells. Cancers (Basel) 2021, 13(10).
32. Dong J, Hu Z, Wu C, Guo H, Zhou B, Lv J, et al. Association analyses identify multiple new lung cancer susceptibility loci and their interactions with smoking in the Chinese population. Nat Genet 2012, 44(8): 895-899.
33. Zhang H, Zhai Y, Hu Z, Wu C, Qian J, Jia W, et al. Genome-wide association study identifies 1p36.22 as a new susceptibility locus for hepatocellular carcinoma in chronic hepatitis B virus carriers. Nat Genet 2010, 42(9): 755-758.
34. Stacey S N, Gudbjartsson D F, Sulem P, Bergthorsson J T, Kumar R, Thorleifsson G, et al. Common variants on 1p36 and 1q42 are associated with cutaneous basal cell carcinoma but not with melanoma or pigmentation traits. Nat Genet 2008, 40(11): 1313-1318.
35. Hu Z, Xia Y, Guo X, Dai J, Li H, Hu H, et al. A genome-wide association study in Chinese men identifies three risk loci for non-obstructive azoospermia. Nat Genet 2011, 44(2): 183-186.
36. Corn P G, Kuerbitz S J, van Noesel M M, Esteller M, Compitello N, Baylin S B, et al. Transcriptional silencing of the p73 gene in acute lymphoblastic leukemia and Burkitt's lymphoma is associated with 5′ CpG island methylation. Cancer Res 1999, 59(14): 3352-3356.
37. Fujita T, Igarashi J, Okawa E R, Gotoh T, Manne J, Kolla V, et al. CHD5, a tumor suppressor gene deleted from 1p36.31 in neuroblastomas. J Natl Cancer Inst 2008, 100(13): 940-949.
38. Gorringe K L, Choong D Y, Williams L H, Ramakrishna M, Sridhar A, Qiu W, et al. Mutation and methylation analysis of the chromodomain-helicase-DNA binding 5 gene in ovarian cancer. Neoplasia 2008, 10(11): 1253-1258.
39. Mulero-Navarro S, Esteller M. Chromatin remodeling factor CHD5 is silenced by promoter CpG island hypermethylation in human cancer. Epigenetics 2008, 3(4): 210-215.
40. Du Z, Li L, Huang X, Jin J, Huang S, Zhang Q, et al. The epigenetic modifier CHD5 functions as a novel tumor suppressor for renal cell carcinoma and is predominantly inactivated by promoter CpG methylation. Oncotarget 2016, 7(16): 21618-21630.
41. Seki N, Ohira M, Nagase T, Ishikawa K, Miyajima N, Nakajima D, et al. Characterization of cDNA clones in size-fractionated cDNA libraries from human brain. DNA Res 1997, 4(5): 345-349.
42. Pang J C, Li K K, Lau K M, Ng Y L, Wong J, Chung N Y, et al. KIAA0495/PDAM is frequently downregulated in oligodendroglial tumors and its knockdown by siRNA induces cisplatin resistance in glioma cells. Brain Pathol 2010, 20(6): 1021-1032.
43. Wu P, Mo Y, Peng M, Tang T, Zhong Y, Deng X, et al. Emerging role of tumor-related functional peptides encoded by lncRNA and circRNA. Mol Cancer 2020, 19(1): 22.
44. Hartford C C R, Lal A. When Long Noncoding Becomes Protein Coding. Mol Cell Biol 2020, 40(6).
45. Huang N, Li F, Zhang M, Zhou H, Chen Z, Ma X, et al. An Upstream Open Reading Frame in Phosphatase and Tensin Homolog Encodes a Circuit Breaker of Lactate Metabolism. Cell Metab 2021, 33(1): 128-144 e129.
46. Alessi D R, James S R, Downes C P, Holmes A B, Gaffney P R, Reese C B, et al. Characterization of a 3-phosphoinositide-dependent protein kinase which phosphorylates and activates protein kinase Balpha. Current biology: CB 1997, 7(4): 261-269.
47. Li B, Cheung P Y, Wang X, Tsao S W, Ling M T, Wong Y C, et al. Id-1 activation of PI3K/Akt/NFkappaB signaling pathway and its significance in promoting survival of esophageal cancer cells. Carcinogenesis 2007, 28(11): 2313-2320.
48. Amiri A, Noei F, Jeganathan S, Kulkarni G, Pinke D E, Lee J M. eEF1A2 activates Akt and stimulates Akt-dependent actin remodeling, invasion and migration. Oncogene 2007, 26(21): 3027-3040.
49. Garnett M J, Edelman E J, Heidorn S J, Greenman C D, Dastur A, Lau K W, et al. Systematic identification of genomic markers of drug sensitivity in cancer cells. Nature 2012, 483(7391): 570-575.
50. Maiuri M C, Tasdemir E, Criollo A, Morselli E, Vicencio J M, Carnuccio R, et al. Control of autophagy by oncogenes and tumor suppressor genes. Cell Death Differ 2009, 16(1): 87-93.
51. Vicinanza M, Korolchuk V I, Ashkenazi A, Puri C, Menzies F M, Clarke J H, et al. PI(5)P regulates autophagosome biogenesis. Mol Cell 2015, 57(2): 219-234.
52. Hasegawa J, Strunk B S, Weisman L S. PI5P and PI(3,5)P2: Minor, but Essential Phosphoinositides. Cell Struct Funct 2017, 42(1): 49-60.
53. Dall'Armi C, Devereaux K A, Di Paolo G. The role of lipids in the control of autophagy. Curr Biol 2013, 23(1): R33-45.
54. Fang Y, Vilella-Bach M, Bachmann R, Flanigan A, Chen J. Phosphatidic acid-mediated mitogenic activation of mTOR signaling. Science 2001, 294(5548): 1942-1945.
55. Li L, Zhang Y, Fan Y, Sun K, Su X, Du Z, et al. Characterization of the nasopharyngeal carcinoma methylome identifies aberrant disruption of key signaling pathways and methylated tumor suppressor genes. Epigenomics 2015, 7(2): 155-173.
56. Li L, Li W, Xiao L, Xu J, Chen X, Tang M, et al. Viral oncoprotein LMP1 disrupts p53-induced cell cycle arrest and apoptosis through modulating K63-linked ubiquitination of p53. Cell Cycle 2012, 11(12): 2327-2336.
57. Li L, Li Z, Zhou S, Xiao L, Guo L, Tao Y, et al. Ubiquitination of MDM2 modulated by Epstein-Barr virus encoded latent membrane protein 1. Virus Res 2007, 130(1-2): 275-280.
58. Li L, Tao Q, Jin H, van Hasselt A, Poon F F, Wang X, et al. The tumor suppressor UCHL1 forms a complex with p53/MDM2/ARF to promote p53 signaling and is frequently silenced in nasopharyngeal carcinoma. Clin Cancer Res 2010, 16(11): 2949-2958.

All patents, patent applications, and other publications, including GenBank Accession Numbers, cited in this application are incorporated by reference in the entirety for all purposes.

TABLE 1
Summary of KIAA0495 promoter methylation
in cell lines and primary tumors

| | Promoter |
|---|---|
| Samples | methylation (%) |
| Carcinoma cell lines | |
| Colorectal Ca (CRC) | 11/11 (100%) |
| Gastric Ca | 9/16 (56%) |
| Head and neck Ca (HNC) | 3/4 (75%) |
| Esophageal squamous | 12/17 (71%) |
| cell Ca (ESCC) | |
| Breast Ca (BrCa) | 5/9 (55%) |
| Bladder Ca | 2/3 |
| Cervical Ca (CxCa) | 2/4 |
| Lung Ca | 2/7 (29%) |
| Hepatocellular Ca (HCC) | 2/13 (15%) |
| Renal Ca (RCC) | 1/9 (11%) |
| Ovarian Ca | 0/2 |
| Prostate Ca | 0/3 |
| Lymphoma | 16/23 (70%) |
| Primary carcinomas | |
| Nasopharyngeal Ca | 28/48 (58%) |
| Esophageal Ca | 7/46 (15%) |
| Gastric Ca | 15/51 (30%) |
| Colon Ca | 14/23 (61%) |
| Breast Ca | 3/40 (7.5%) |
| Nose swab samples | 7/16 (44%) |
| from NPC patients | |
| Immortalized normal | 0/9 |
| epithelial cell lines | |
| Normal tissues | |
| Normal nasopharyngeal tissues | 0/6 |
| Normal esophageal tissues | 0/7 |
| Normal gastric tissues | 0/4 |
| Surgical-margin esophageal | 0/46 |
| tissue from | |
| esophageal Ca patients | |
| Surgical-margin tissue | 2w/12 (17%) |
| from CRC patients | |

w: weak methylation

TABLE 2
Reduced KIAA0495 copy numbers in multiple tumors.

| | Median of copy | | |
|---|---|---|---|
| | number units | Sample | |
| Tissue type | (log₂) | number | p-value |
| Blood | 0.000 | 702 | |
| Breast | 0.001 | 111 | |
| Invasive ductal | −0.079 | 647 | 1.64E−44 |
| breast carcinoma | | | |
| Blood | 0.005 | 183 | |
| Lung | −0.002 | 268 | |
| Squamous cell | −0.046 | 204 | 2.48E−9 |
| lung carcinoma | | | |
| Blood | 0.009 | 351 | |
| Colon | −0.003 | 79 | |
| Colon | −0.097 | 212 | 1.30E−20 |
| adenocarcinoma | | | |
| Blood | 0.001 | 75 | |
| Gastric tissue | −0.008 | 68 | |
| Gastric | −0.041 | 81 | 3.38E−5 |
| adenocarcinoma | | | |
| Blood | 0.004 | 309 | |
| Brain | 0.012 | 12 | |
| Glioblastoma | −0.013 | 472 | 6.96E−11 |

TABLE 3
Reduced KIAA0495 expression in tumors
compared to normal tissues.

| | Median of | | |
|---|---|---|---|
| | expression | | |
| | intensity | Sample | |
| Tissue type | (log₂) | number | p-value |
| Lung | 0.245 | 65 | |
| Lung adenocarcinoma | −0.008 | 45 | 2.91E−4 |
| Squamous cell | −0.579 | 27 | 1.38E−9 |
| lung carcinoma | | | |
| Esophagus | −0.864 | 53 | |
| ESCC | −0.969 | 53 | 7.97E−6 |
| Breast | 0.007 | 61 | |
| Invasive ductal | −0.496 | 392 | 3.34E−6 |
| breast carcinoma | | | |
| Pancreas | −0.760 | 39 | |
| Pancreatic adenocarcinoma | −1.020 | 39 | 1.94E−5 |
| Colon | 0.866 | 24 | |
| Colorectal Adenocarcinoma | −0.119 | 45 | 2.76E−10 |
| Colorectal Carcinoma | 0.352 | 35 | 3.20E−5 |
| Gastric mucosa | 1.744 | 31 | |
| Gastric adenocarcinoma | 1.079 | 26 | 5.19E−5 |
| Bladder | 2.814 | 68 | |
| Superficial bladder | 1.690 | 126 | 1.04E−12 |
| cancer | | | |
| Infiltrating bladder | 1.504 | 62 | 1.45E−11 |
| carcinoma | | | |
| Cervix uteri | 0.626 | 5 | |
| Cervical squamous | −0.119 | 40 | 2.47E−5 |
| cell carcinoma | | | |
| Ovarian epithelium | 0.618 | 10 | |
| Ovarian carcinoma | −0.592 | 185 | 2.43E−4 |
| Brain | 1.959 | 23 | |
| Oligodendroglioma | 1.036 | 50 | 2.01E−6 |

ESCC: esophageal squamous cell carcinoma

TABLE 4
Association of KIAA0495 downregulation with clinicopathologic
features of bladder cancer patients.

| | | KIAA0495 expression | | |
|---|---|---|---|---|
| Clinical features | Total case | Low (%) | High (%) | p value |
| Sex | | | | .119 |
| Male | 192 | 41.7 | 58.3 | |
| Female | 48 | 54.2 | 45.8 | |
| Age | | | | .017 |
| <=65 years old | 102 | 35.3 | 64.7 | |
| >65 years old | 138 | 50.7 | 49.3 | |
| Progression | | | | .000 |
| Yes | 83 | 66.3 | 33.7 | |
| No | 82 | 34.1 | 65.9 | |
| Grade | | | | .119 |
| Low | 105 | 45.7 | 54.3 | |
| High | 60 | 50.3 | 49.7 | |
| T Stage | | | | .672 |
| Ta | 24 | 37.5 | 62.5 | |
| T1 | 80 | 50.0 | 50.0 | |
| T2 | 31 | 54.8 | 45.2 | |
| T3 | 19 | 57.9 | 42.1 | |
| T4 | 11 | 54.5 | 45.5 | |

TABLE 5
Correlation between SP0495 protein expression and clinicopathological
characteristics in colorectal cancer

| | | SP0495 | expression | | |
|---|---|---|---|---|---|
| | variables | low | high | total | p-value |
| Sex | | | | | 0.642 |
| | male | 17 | 26 | 43 | |
| | Female | 12 | 25 | 37 | |
| Age (year) | | | | | 0.802 |
| | ≤59 | 10 | 15 | 25 | |
| | >59 | 19 | 36 | 55 | |
| Grade | | | | | 1 |
| | I/II | 24 | 41 | 65 | |
| | III | 5 | 10 | 15 | |
| Tumor size | | | | | 0.035 |
| | ≤4.8 cm | 7 | 25 | 32 | |
| | >4.8 cm | 22 | 26 | 48 | |
| T stage | | | | | 0.488 |
| | T1-T3 | 12 | 26 | 38 | |
| | T4 | 17 | 25 | 42 | |
| N stage | | | | | 0.808 |
| | N0 | 20 | 33 | 53 | |
| | N1/N2 | 9 | 18 | 27 | |
| TNM stage | | | | | 0.632 |
| | I/II | 20 | 32 | 52 | |
| | III/IV | 9 | 19 | 28 | |

TABLE 6
Mutation screening for KIAA0495-ORF2 in tumor cell lines.

| Tumor type | Cell line | Mutation | Expression |
|---|---|---|---|
| HNC | HONE1 | No | + |
| Esophageal Ca | HKESC1 | No | + |
| | HKESC2 | No | + |
| | HKESC3 | No | + |
| | EC18 | No | + |
| | KYSE70 | No | + |
| | KYSE180 | No | + |
| | KYSE270 | No | + |
| | KYSE510 | No | + |
| | KYSE520 | No | + |
| | SLMT-1 | No | + |
| Gastric Ca | YCC1 | No | + |
| | YCC2 | No | + |
| | YCC3 | No | + |
| | YCC6 | No | + |
| | YCC7 | No | + |
| | YCC11 | No | + |
| Colon Ca | HCT116 | No | -ve |
| | SW480 | No | -ve |

Coding exons of KIAA0495-ORF2 were amplified using pfu high-fidelity DNA polymerase, with primers seq1/seq6 for exon 1, and primers seq5/seq4 for exon 2, respectively, followed by direct sequencing of PCR products. Sequencing results were aligned to the GenBank reference sequence (NM_207306). Expression status of KIAA0495 in examined cell lines is indicated, +: expression; -ve: without endogenous KIAA0495 expression.

TABLE 7
Primers used in this study

| Usage | Primer Name | Primer sequences (5′-3′) |
|---|---|---|
| RT-PCR | KIAA0495F | ACTGCTGCAGAGTGTGGTG |
| | KIAA0495R | ACTTGCTTGTGCAAGAACTG |
| | PIK3CAF | CGTAAGTGTTACTCAAGAAGC |
| | PIK3CAR | CAAATTCACACACTGGCATGC |
| | PIK3CBF | CTCCATACCTGTGGATTTCC |
| | PIK3CBR | GCAGTCTGATTCACACATGC |
| | PDK1F | CCCCATCAATGGTGAGGAC |
| | PDK1R | CAAACTTGAAGTCCTCAGGC |
| | ID1F | TGGAGATTCTCCAGCACGTC |
| | ID1R | ATGCGATCGTCCGCAGGAAC |
| | eEF1A2F | AGTTCGAGACCACCAAGTAC |
| | eEF1A2R | CACGATGAGCTGCTTCACAC |
| | CTNNBIF | TCT GAG GAG CAG CTT CAG TC |
| | CTNNBIR | GCT CGA GTC ATT GCA TAC TG |
| | MYCF | CTCTCCGTCCTCGGATTCTC |
| | MYCR | GCCTCCAGCAGAAGGTGATC |
| | CCND1F | TGCTGCGAAGTGGAAACCAT |
| | CCND1R | GCGGTCCAGGTAGTTCATG |
| | MMP7F | GACTCACCGTGCTGTGTGC |
| | MMP7R | ACATTCCAGTTATAGGTAGG |
| | MITFF | AGCCATGCAGTCCGAATCG |
| | MITFR | ATCTGCTCACGCATGAGTTG |
| | TWIST1F | TCGACTTCCTCTACCAGGTC |
| | TWIST1R | CCAGAGTCTCTAGACTGTCC |
| | STAT5BF | ACAAGCTCAGCAGCTCCAAG |
| | STAT5BR | TGGGTGGCCTTAATGTTCTC |
| | HMGA2F | CACTTCAGCCCAGGGACAAC |
| | HMGA2R | CTAGGTCTGCCTCTTGGCCG |
| | IKBKEF | CAGCCAATTACCTGTGGCAC |
| | IKBKER | TCCACCGCAAAGAGCTTGAC |
| | YAP1F | CATGAGGCTCCGGAAGCTGC |
| | YAP1R | CTGTCGAAGATGCTGAGCTG |
| | GAPDH33 | GATGACCTTGCCCACAGCCT |
| | GAPDH55 | ATCTCTGCCCCCTCTGCTGA |
| MSP | KIAA0495ml | TCGTTAGATTTTTACGTTTCGC |
| | KIAA0495m2 | ACAACCCGAATCCGAAAACG |
| | KIAA0495u1 | GATTTGTTAGATTTTTATGTTTTGT |
| | KIAA0495u2 | CAACAACCCAAATCCAAAAACA |
| BGS | KIAA0495BGS1 | AGTATTTGGGTGTAGGTGTATT |
| | KIAA0495BGS3 | AACRAAAAAAAAATCCCTTCCTA |
| Cloning | KIAA0495-CF | CACCATGGATTACAAGGATGACGACG |
| | | ATAAGTGTCTTTTGTCCAGC |
| | KIAA0495-CR | TCAAAGTGCCGCTGGTCGTTGAG |
| Sequencing | KIAA0495-seq1 | TGCTGCCTTATCACAAGCCA |
| | KIAA0495-seq4 | CGGTGATCCAGTGGATCCT |
| | KIAA0495-seq5 | AATCGTGAGGGATGCTCTCC |
| | KIAA0495-seq6 | ATTCAGCAGCACAAGCATGG |

TABLE 8
Antibodies used in this study

| Antibodies | Source | Identifier |
|---|---|---|
| anti-mouse IgG F(ab)2 | DAKO | F0313 |
| anti-mouse IgG-Alexa | Cell Signaling | 4409 |
| Fluor 555-F(ab′)2 | | |
| anti-rabbit IgG-Alexa | Cell Signaling | 4413 |
| Fluor 555-F(ab′)2 | | |
| anti-mouse IgG-Alexa | ThermoFisher | A-11059 |
| Fluor 488-F(ab′)2 | | |
| anti-rabbit IgG-Alexa | ThermoFisher | A-11070 |
| Fluor 488-F(ab′)2 | | |
| anti-mouse IgG-HRP | DAKO | P0161 |
| anti-rabbit IgG-HRP | DAKO | P0448 |
| a-tubulin | Lab Vision | MS-581 |
| Active-β-catenin | Upstate | 05-665 |
| AKT (pan) | Cell Signaling | 4691 |
| ATG5 | Cell Signaling | 12994 |
| β-catenin | DAKO | M3539 |
| β-actin (AC-74) | Sigma-Aldrich | A2228 |
| CCND1 | Cell Signaling | 2978 |
| BCL2 | Cell Signaling | 4223 |
| BECN1 | Cell Signaling | 3495 |
| CDK4 | Cell Signaling | 12790 |
| CDK6 | Cell Signaling | 13331 |
| cleaved caspase-3 | Cell Signaling | 9661 |
| cleaved PARP | Cell Signaling | 9541 |
| c-Myc | Cell Signaling | 13987 |
| Cyclin D1 | Cell Signaling | 55506 |
| Cyclin B1 | Cell Signaling | 12231 |
| Flag | Sigma-Aldrich | F3165 |
| GAPDH | Millipore | MAB374 |
| GSK-3β | Cell Signaling | 9315 |
| HA | Santa Cruz | sc-7392 |
| His | Santa Cruz | sc-8036 |
| INSR | Santa Cruz | sc-57342 |
| KIAA0495/SP0495 | Origene | TA503634 |
| LC3A/B | Cell Signaling | 12741 |
| mTOR | Cell Signaling | 2983 |
| MMP7 | Thermo Scientific | MS-813-PO |
| NF-κB pathway | Cell Signaling | 9936 |
| antibody sampler kit | | |
| p21 | Calbiochem | OP64 |
| p53 | DAKO | M7001 |
| PARP | Cell Signaling | 9542 |
| PI(3, 4, 5)P3 | Echelon Biosceinces | Z-P345b |
| SQSTM1/p62 | Cell Signaling | 8025 |
| phospho-AKT (Ser473) | Cell Signaling | 4060 |
| phospho-GSK-3β | Cell Signaling | 5558 |
| phospho-mTOR (Ser2448) (D9C2) | Cell Signaling | 5536 |
| phospho-p53 (Ser15) | Cell Signaling | 2461 |
| phospho-Rb | Cell Signaling | 8516 |
| Rb | Cell Signaling | 9313 |

1. A method for assessing risk for cancer in a subject, comprising the steps of:
(a) measuring expression level of SP0495 in a sample taken from the subject,
(b) comparing the expression level obtained in step (a) with a standard control, and
(c) determining the subject, who has a reduced SP0495 expression level compared with the standard control, as having an increased risk for cancer.

2. The method of claim 1, wherein the sample is a blood sample or a colorectal, gastric, breast, cervical, bladder, or esophageal tissue sample.

3. The method of claim 1, wherein the expression level of SP0495 is SP0495 protein level.

4. The method of claim 1, wherein the expression level of SP0495 is SP0495 mRNA level.

5. The method of claim 3, wherein step (a) comprises an immunoassay using an antibody that specifically binds the SP0495 protein.

6. The method of claim 4, wherein step (a) comprises an amplification reaction.

7. The method of claim 6, wherein the amplification reaction is a polymerase chain reaction (PCR).

8. The method of claim 7, wherein the PCR is a reverse transcriptase-PCR (RT-PCR).

9-12. (canceled)

13. A method for assessing risk for cancer in a subject, comprising the steps of:
(a) treating DNA from a sample taken from the subject with an agent that differentially modifies methylated and unmethylated DNA;
(b) determining number of methylated CpGs in a genomic sequence, which is SEQ ID NO:3 or a fragment thereof comprising at least 10 CpGs, and
(c) comparing the number of methylated CpGs from step (b) with the number of methylated CpGs in the genomic sequence from a non-cancer sample of the corresponding type and processed through steps (a) and (b); and
(d) determining the subject, whose sample contains more methylated CpGs in the genomic sequence determined in step (b) compared to the number of methylated CpGs with the number of methylated CpGs in the genomic sequence from a non-cancer sample and processed through steps (1) to (3), as having an increased risk for cancer compared with a healthy subject not diagnosed with cancer.

14. The method of claim 13, wherein the genomic sequence is SEQ ID NO:3.

15. The method of claim 13, wherein the agent that differentially modifies methylated DNA and unmethylated DNA is an enzyme that preferentially cleaves methylated DNA, an enzyme that preferentially cleaves unmethylated DNA, or a bisulfite.

16. The method of claim 13, wherein step (b) comprises an amplification reaction.

17. The method of claim 16, wherein the amplification reaction is a PCR.

18. A method for assessing likelihood of mortality from cancer in a patient who has received a cancer diagnosis, comprising the steps of:
(a) treating DNA from a cancer tissue sample taken from a first patient with an agent that differentially modifies methylated and unmethylated DNA;
(b) determining number of methylated CpGs in a genomic sequence, which is SEQ ID NO:3 or a fragment thereof comprising at least 10 CpGs, and
(c) comparing the number of methylated CpGs from step (b) with the number of methylated CpGs in the genomic sequence from another cancer tissue sample of the same type obtained from a second patient and processed through steps (a) and (b); and
(d) determining the first patient, whose cancer tissue sample contains more methylated CpGs in the genomic sequence determined in step (b) compared to the number of methylated CpGs with the number of methylated CpGs in the genomic sequence from the cancer tissue sample obtained from the second patient and processed through steps (1) to (3), as having an increased likelihood of mortality from cancer compared with the second patient.

19. The method of claim 18, wherein the genomic sequence is SEQ ID NO:3.

20. The method of claim 18, wherein the agent that differentially modifies methylated DNA and unmethylated DNA is an enzyme that preferentially cleaves methylated DNA, an enzyme that preferentially cleaves unmethylated DNA, or a bisulfite.

21. The method of claim 18, wherein step (b) comprises an amplification reaction.

22. The method of claim 21, wherein the amplification reaction is a PCR.

23. A kit for detecting cancer in a subject, comprising (1) a standard control that provides an average amount of SP0495 protein or SP0495 mRNA; and (2) an agent that specifically and quantitatively identifies SP0495 protein or SP0495 mRNA.

24-28. (canceled)

29. A method for inhibiting growth of a cancer cell, comprising contacting the cancer cell with an effective amount of a polypeptide comprising the amino acid sequence set forth in SEQ ID NO:1 or a nucleic acid comprising a polynucleotide sequence encoding SEQ ID NO:1.

30-33. (canceled)


SEQUENCE LISTING (53 sequences)

SEQ ID NO: 1 - protein, 201 aa, synthetic construct
  REGION 1..201: note = Description of sequence: SP0495 protein
MCLLSSSAASDLAATSLTAHPCHPPLPRQPQPQLILSGSPSIGTVPPPAVGHTSLLPVNPPAISTTTSDLSAREDATPSASTGHLSVFPAFQVKIPAVPSEQTSQSFSEASHRVLPGGGPRCSRDFGAGVAGHLGLGIFGVGFGSPALLQSVVDENSCCLLYVVEDQLCDVEQAFRAEHLGQHQSGSGTGCRHCPQRPAAL

SEQ ID NO: 2 - DNA, 606 bp, synthetic construct
  misc_feature 1..606: note = Description of sequence: coding sequencing for SP0495 protein
  source 1..606: mol_type = other DNA
atgtgtcttttgtccagctcagccgcctcggatctcgctgccaccagccttactgcacacccgtgccacccgcccttgccccgtcagcctcagcctcagctcattctctcaggcagtcccagcattggcacggtacctcctcccgctgtgggccacacgtctctgctccctgtcaaccctcctgccatcagcaccaccaccagcgacttgtctgcccgggaggatgcaacaccatctgcctccaccggccacctttcagtgtttcctgctttccaagtaaagataccagcagtgccctcagagcagaccagccagagtttttctgaagcctcccacagggtgctccccggaggaggcccgagatgctctcgtgactttggagccggggtggctggccaccttggcctgggcatctttggggtgggcttcgggagcccggcactgctgcagagtgtggtggatgagaacagctgctgcttgctgtacgtggtggaggaccagctgtgtgatgtggagcaagccttcagagctgagcatttgggccaacaccagagtggttcgggaacaggatgcagacattgtcctcaacgaccagcggcactttga

SEQ ID NO: 3 - DNA, 488 bp, synthetic construct
  misc_feature 1..488: note = Description of sequence: genomic DNA sequence for KIAA0495 promoter CGI
  source 1..488: mol_type = other DNA
agtcagcacctgggtgcaggtgcatctgggacccgtcagatctctacgccccgcgcaggctccgcccccacgcccaagaccccgccccgcgcccggagcctccctcgacttggccctgggtctgcaccgaaggccccacccctgccctcgcccagccgcgaccccgcgcttccggatccgggttgttgcgggatctcacaggctttaaaccgcggcgccgcggcgcccgggtgtggatccctagatgggagccggggatgggccgggtgcctggtgggtggcagtcggggctgacggcggcggcactttgccgcctcaggccctggacaccttcaccccgccgcctgcccaggcgggccggccctgcccgtccaccggccgccgagagtccccggccttgggtccccggggccgctgactggcctcggtcacctcccggggaaggctcccgcgcctccatctgcccccgcaggaagggaccctcttct

SEQ ID NO: 4 - DNA, 218 bp, synthetic construct
  misc_feature 1..218: note = Description of sequence: KIAA0495/TP73-as1
  source 1..218: mol_type = other DNA
tcattggaacatacactttattatgttacaaaaacaaaaatccccactgaaacacagctaaaaaaataacacattttcccaagattacattaccaaaaacagttgttatgtcattggagggcgtccattaatactgctcggagaagcacgatcttacacgaaaaacacggatgggatttcgttttcaccttaaagcattaaagtgctttaactggtaa

SEQ ID NO: 5 - DNA, 218 bp, synthetic construct; identical to SEQ ID NO: 4
  misc_feature 1..218: note = Description of sequence: TP73
  source 1..218: mol_type = other DNA

SEQ ID NO: 6 - protein, 201 aa, Pan troglodytes
  REGION 1..201: note = Description of sequence: SP0495 protein
MCLLSSSATSDLAATSLTAHPCHPPLPRQPQPQLILSGSPSIGTVPPPAVGHTSLLPVNPTAISTTTSDLSAQEDATPSASTGHLSVFPAFQVKIPAVPAEQTSQRFSEASHRVLPGGGPRCSRDFGAGVAGHLGLGIFGVGFGSPALLQSVVDENSCCLLYVVEDQLCDVERAFRAEHLGQHQSGSGTGCRHCPQRPAAL

SEQ ID NO: 7 - protein, 199 aa, Chlorocebus sp.
  REGION 1..199: note = Description of sequence: SP0495 protein
  VARIANT 179: note = X is any amino acid
MCLLSSSAASDLTATGLHAHPRHQPLPRQPQPQLILSGSPSIGMVPPPAVGLTSLLPVNPTAISITTSDLSAQEDATPSTSTGHLSVFSAFQVKTPAVPSERTSQRFSEASHRLLRGGGLRCCHDFGARVADHLGLAIFGVGFGSPALLPSVVDENDCCLLYVVELCGVERAFRAEHLXQHQSGSGTGCRHCPQRPAAL

SEQ ID NO: 8 - DNA, 19 bp, synthetic construct
  misc_feature 1..19: note = Description of sequence: RT-PCRprimer, Primer Name: KIAA0495F
  source 1..19: mol_type = other DNA
actgctgcagagtgtggtg

SEQ ID NO: 9 - DNA, 20 bp, synthetic construct
  misc_feature 1..20: note = Description of sequence: RT-PCRprimer, Primer Name: KIAA0495R
  source 1..20: mol_type = other DNA
acttgcttgtgcaagaactg

SEQ ID NO: 10 - DNA, 21 bp, synthetic construct
  misc_feature 1..21: note = Description of sequence: RT-PCRprimer, Primer Name: PIK3CAF
  source 1..21: mol_type = other DNA
cgtaagtgttactcaagaagc

SEQ ID NO: 11 - DNA, 21 bp, synthetic construct
  misc_feature 1..21: note = Description of sequence: RT-PCRprimer, Primer Name: PIK3CAR
  source 1..21: mol_type = other DNA
caaattcacacactggcatgc

SEQ ID NO: 12 - DNA, 20 bp, synthetic construct
  misc_feature 1..20: note = Description of sequence: RT-PCRprimer, Primer Name: PIK3CBF
  source 1..20: mol_type = other DNA
ctccatacctgtggatttcc

SEQ ID NO: 13 - DNA, 20 bp, synthetic construct
  misc_feature 1..20: note = Description of sequence: RT-PCRprimer, Primer Name: PIK3CBR
  source 1..20: mol_type = other DNA
gcagtctgattcacacatgc

SEQ ID NO: 14 - DNA, 19 bp, synthetic construct
  misc_feature 1..19: note = Description of sequence: RT-PCRprimer, Primer Name: PDK1F
  source 1..19: mol_type = other DNA
ccccatcaatggtgaggac

SEQ ID NO: 15 - DNA, 20 bp, synthetic construct
  misc_feature 1..20: note = Description of sequence: RT-PCRprimer, Primer Name: PDK1R
  source 1..20: mol_type = other DNA
caaacttgaagtcctcaggc

SEQ ID NO: 16 - DNA, 20 bp, synthetic construct
  misc_feature 1..20: note = Description of sequence: RT-PCRprimer, Primer Name: ID1F
  source 1..20: mol_type = other DNA
tggagattctccagcacgtc

SEQ ID NO: 17 - DNA, 20 bp, synthetic construct
  misc_feature 1..20: note = Description of sequence: RT-PCRprimer, Primer Name: ID1R
  source 1..20: mol_type = other DNA
atgcgatcgtccgcaggaac

SEQ ID NO: 18 - DNA, 20 bp, synthetic construct
  misc_feature 1..20: note = Description of sequence: RT-PCRprimer, Primer Name: eEF1A2F
  source 1..20: mol_type = other DNA
agttcgagaccaccaagtac

SEQ ID NO: 19 - DNA, 20 bp, synthetic construct
  misc_feature 1..20: note = Description of sequence: RT-PCRprimer, Primer Name: eEF1A2R
  source 1..20: mol_type = other DNA
cacgatgagctgcttcacac

SEQ ID NO: 20 - DNA, 20 bp, synthetic construct
  misc_feature 1..20: note = Description of sequence: RT-PCRprimer, Primer Name: CTNNB1F
  source 1..20: mol_type = other DNA
tctgaggagcagcttcagtc

SEQ ID NO: 21 - DNA, 20 bp, synthetic construct
  misc_feature 1..20: note = Description of sequence: RT-PCRprimer, Primer Name: CTNNB1R
  source 1..20: mol_type = other DNA
gctcgagtcattgcatactg

SEQ ID NO: 22 - DNA, 20 bp, synthetic construct
  misc_feature 1..20: note = Description of sequence: RT-PCRprimer, Primer Name: MYCF
  source 1..20: mol_type = other DNA
ctctccgtcctcggattctc

SEQ ID NO: 23 - DNA, 20 bp, synthetic construct
  misc_feature 1..20: note = Description of sequence: RT-PCRprimer, Primer Name: MYCR
  source 1..20: mol_type = other DNA
gcctccagcagaaggtgatc

SEQ ID NO: 24 - DNA, 20 bp, synthetic construct
  misc_feature 1..20: note = Description of sequence: RT-PCRprimer, Primer Name: CCND1F
  source 1..20: mol_type = other DNA
tgctgcgaagtggaaaccat

SEQ ID NO: 25 - DNA, 19 bp, synthetic construct
  misc_feature 1..19: note = Description of sequence: RT-PCRprimer, Primer Name: CCND1R
  source 1..19: mol_type = other DNA
gcggtccaggtagttcatg

SEQ ID NO: 26 - DNA, 19 bp, synthetic construct
  misc_feature 1..19: note = Description of sequence: RT-PCRprimer, Primer Name: MMP7F
  source 1..19: mol_type = other DNA
gactcaccgtgctgtgtgc

SEQ ID NO: 27 - DNA, 20 bp, synthetic construct
  misc_feature 1..20: note = Description of sequence: RT-PCRprimer, Primer Name: MMP7R
  source 1..20: mol_type = other DNA
acattccagttataggtagg

SEQ ID NO: 28 - DNA, 19 bp, synthetic construct
  misc_feature 1..19: note = Description of sequence: RT-PCRprimer, Primer Name: MITFF
  source 1..19: mol_type = other DNA
agccatgcagtccgaatcg

SEQ ID NO: 29 - DNA, 20 bp, synthetic construct
  misc_feature 1..20: note = Description of sequence: RT-PCRprimer, Primer Name: MITFR
  source 1..20: mol_type = other DNA
atctgctcacgcatgagttg

SEQ ID NO: 30 - DNA, 20 bp, synthetic construct
  misc_feature 1..20: note = Description of sequence: RT-PCRprimer, Primer Name: TWIST1F
  source 1..20: mol_type = other DNA
tcgacttcctctaccaggtc

SEQ ID NO: 31 - DNA, 20 bp, synthetic construct
  misc_feature 1..20: note = Description of sequence: RT-PCRprimer, Primer Name: TWIST1R
  source 1..20: mol_type = other DNA
ccagagtctctagactgtcc

SEQ ID NO: 32 - DNA, 20 bp, synthetic construct
  misc_feature 1..20: note = Description of sequence: RT-PCRprimer, Primer Name: STAT5BF
  source 1..20: mol_type = other DNA
acaagctcagcagctccaag

SEQ ID NO: 33 - DNA, 20 bp, synthetic construct
  misc_feature 1..20: note = Description of sequence: RT-PCRprimer, Primer Name: STAT5BR
  source 1..20: mol_type = other DNA
tgggtggccttaatgttctc

SEQ ID NO: 34 - DNA, 20 bp, synthetic construct
  misc_feature 1..20: note = Description of sequence: RT-PCRprimer, Primer Name: HMGA2F
  source 1..20: mol_type = other DNA
cacttcagcccagggacaac

SEQ ID NO: 35 - DNA, 20 bp, synthetic construct
  misc_feature 1..20: note = Description of sequence: RT-PCRprimer, Primer Name: HMGA2R
  source 1..20: mol_type = other DNA
ctaggtctgcctcttggccg

SEQ ID NO: 36 - DNA, 20 bp, synthetic construct
  misc_feature 1..20: note = Description of sequence: RT-PCRprimer, Primer Name: IKBKEF
  source 1..20: mol_type = other DNA
cagccaattacctgtggcac

SEQ ID NO: 37 - DNA, 20 bp, synthetic construct
  misc_feature 1..20: note = Description of sequence: RT-PCRprimer, Primer Name: IKBKER
  source 1..20: mol_type = other DNA
tccaccgcaaagagcttgac

SEQ ID NO: 38 - DNA, 20 bp, synthetic construct
  misc_feature 1..20: note = Description of sequence: RT-PCRprimer, Primer Name: YAP1F
  source 1..20: mol_type = other DNA
catgaggctccggaagctgc

SEQ ID NO: 39 - DNA, 20 bp, synthetic construct
  misc_feature 1..20: note = Description of sequence: RT-PCRprimer, Primer Name: YAP1R
  source 1..20: mol_type = other DNA
ctgtcgaagatgctgagctg

SEQ ID NO: 40 - DNA, 20 bp, synthetic construct
  misc_feature 1..20: note = Description of sequence: RT-PCRprimer, Primer Name: GAPDH33
  source 1..20: mol_type = other DNA
gatgaccttgcccacagcct

SEQ ID NO: 41 - DNA, 20 bp, synthetic construct
  misc_feature 1..20: note = Description of sequence: RT-PCRprimer, Primer Name: GAPDH55
  source 1..20: mol_type = other DNA
atctctgccccctctgctga

SEQ ID NO: 42 - DNA, 22 bp, synthetic construct
  misc_feature 1..22: note = Description of sequence: MSPprimer, Primer Name: KIAA0495m1
  source 1..22: mol_type = other DNA
tcgttagatttttacgtttcgc

SEQ ID NO: 43 - DNA, 20 bp, synthetic construct
  misc_feature 1..20: note = Description of sequence: MSPprimer, Primer Name: KIAA0495m2
  source 1..20: mol_type = other DNA
acaacccgaatccgaaaacg

SEQ ID NO: 44 - DNA, 25 bp, synthetic construct
  misc_feature 1..25: note = Description of sequence: MSPprimer, Primer Name: KIAA0495u1
  source 1..25: mol_type = other DNA
gatttgttagatttttatgttttgt

SEQ ID NO: 45 - DNA, 22 bp, synthetic construct
  misc_feature 1..22: note = Description of sequence: MSPprimer, Primer Name: KIAA0495u2
  source 1..22: mol_type = other DNA
caacaacccaaatccaaaaaca

SEQ ID NO: 46 - DNA, 22 bp, synthetic construct
  misc_feature 1..22: note = Description of sequence: BGSprimer, Primer Name: KIAA0495BGS1
  source 1..22: mol_type = other DNA
agtatttgggtgtaggtgtatt

SEQ ID NO: 47 - DNA, 23 bp, synthetic construct
  misc_feature 1..23: note = Description of sequence: BGSprimer, Primer Name: KIAA0495BGS3
  source 1..23: mol_type = other DNA
aacraaaaaaaaatcccttccta

SEQ ID NO: 48 - DNA, 46 bp, synthetic construct
  misc_feature 1..46: note = Description of sequence: Cloningprimer, Primer Name: KIAA0495-CF
  source 1..46: mol_type = other DNA
caccatggattacaaggatgacgacgataagtgtcttttgtccagc

SEQ ID NO: 49 - DNA, 23 bp, synthetic construct
  misc_feature 1..23: note = Description of sequence: Cloningprimer, Primer Name: KIAA0495-CR
  source 1..23: mol_type = other DNA
tcaaagtgccgctggtcgttgag

SEQ ID NO: 50 - DNA, 20 bp, synthetic construct
  misc_feature 1..20: note = Description of sequence: Sequencingprimer, Primer Name: KIAA0495-seq1
  source 1..20: mol_type = other DNA
tgctgccttatcacaagcca

SEQ ID NO: 51 - DNA, 19 bp, synthetic construct
  misc_feature 1..19: note = Description of sequence: Sequencingprimer, Primer Name: KIAA0495-seq4
  source 1..19: mol_type = other DNA
cggtgatccagtggatcct

SEQ ID NO: 52 - DNA, 20 bp, synthetic construct
  misc_feature 1..20: note = Description of sequence: Sequencingprimer, Primer Name: KIAA0495-seq5
  source 1..20: mol_type = other DNA
aatcgtgagggatgctctcc

SEQ ID NO: 53 - DNA, 20 bp, synthetic construct
  misc_feature 1..20: note = Description of sequence: Sequencingprimer, Primer Name: KIAA0495-seq6
  source 1..20: mol_type = other DNA
attcagcagcacaagcatgg